(19) United States
(12) Patent Application Publication
(10) Pub. No.: US 2026/0217712 A1
(43) Pub. Date: Jul. 30, 2026

(54) SOLID STATE FORMS OF MNK INHIBITORS

(71) Applicants: 4E Therapeutics, Inc., Austin, TX (US); Board of Regents, The University of Texas System, Austin, TX (US)

(72) Inventors: Theodore J. Price, Austin, TX (US); James J. Sahn, Austin, TX (US); Daniel Rixson, Godmanchester, Cambridgeshire (GB); Osama Suleiman, Cambridge, Cambridgeshire (GB); Arlindo Lucas Castelhano, Vagos (PT); Garry Robert Smith, Elverson, PA (US)

(21) Appl. No.: 19/145,296

(22) PCT Filed: Jan. 3, 2024
(86) PCT No.: PCT/US2024/010110

§ 371 Date: Jul. 2, 2025

Related U.S. Application Data
(60) Provisional application No. 63/478,409, filed on Jan. 4, 2023.

(51) Int. Cl. C07D 471/10

(52) U.S. Cl. CPC C07D 471/10

(57) ABSTRACT

The present disclosure relates to solid forms of a compound having the following Structure (I): or a tautomer thereof. The present disclosure is also directed to methods of making and using a compound of Structure (I).

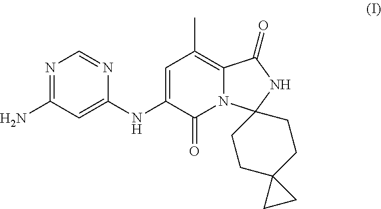 [SMILES: Cc1cc(Nc2cc(N)ncn2)c(=O)n2c1C(=O)NC21CCC2(CC2)CC1]

### RELATED APPLICATIONS

This application claims the benefit of and priority to U.S. Application No. 63/478,409, filed Jan. 4, 2023, the entire contents of which are hereby incorporated by reference.

### STATEMENT OF GOVERNMENT INTEREST

This invention was made with government support under grant No. 1U44NS115692-01 awarded by the National Institutes of Health. The government has certain rights in the invention.

### TECHNICAL FIELD

Embodiments of the present disclosure are generally directed to solid forms of compounds and methods for their preparation and use as therapeutic or prophylactic agents, for example for treatment of various diseases and disorders (e.g., inflammation, neuropathic pain, migraine pain, etc.).

### BACKGROUND

Inadequate treatment of pain is a devastating health problem in the United States. One third of all Americans suffer from some form of chronic pain and a third of these have pain that is resistant to current medical therapies. The economic impact of pain is equally large at approximately $100 billion annually. Opioid or narcotic analgesics, typified by morphine, are the most effective treatments for acute and chronic severe pain. However, their clinical utility is often hampered by the development of analgesic tolerance which requires escalating doses to achieve equivalent pain relief. Furthermore, these drugs are often ineffective for neuropathic pain treatment. This complex pathophysiological cycle represents a critical barrier to the quality of life of these patients due to the resulting drug-induced sedation, reduced physical activity, constipation, respiratory depression, high potential for addiction, and other side effects.

Neuropathic pain typically develops over time and may benefit from therapies that interfere with pathways involved in its development and/or continuation.

Disease or damage causing neuropathic pain may affect the central nervous system (CNS), the peripheral nervous system, or both (as opposed to causes of nociceptive pain, which affect the peripheral nervous system only). Common causes of neuropathic pain include spinal cord injury, multiple sclerosis, central nervous system ischemia, spinal nerve disease, diabetes, other metabolic disorders, herpes zoster infection, HIV-related neuropathies, nutritional deficiencies, toxins, remote manifestations of malignancies, immune mediated disorders, physical trauma to a nerve trunk such as during surgery, peripheral ischemia, peripheral nerve lesions, nerve compression, chemotherapy or other drug-induced nerve damage, radiation injury, arthritis, autoimmune disease, and infection in an area near the affected nerves.

Neuropathic pain often involves abnormal nociceptor sensitivity. Nociceptors are specialized neurons that detect pain. Nociceptor sensitivity is not fixed; it can change over time. Some causes of neuropathic pain affect nociceptor sensitivity by inducing “peripheral sensitization.” Peripheral sensitization includes spontaneous pathological activity, abnormal excitability, heightened sensitivity to chemical stimuli, heightened sensitivity to thermal stimuli, heightened sensitivity to mechanical stimuli, and any combinations of these.

Disruption of peripheral sensitization, either by reducing or preventing such peripheral sensitization in the first place or by reducing the degree of already-developed peripheral sensitization, may therefore treat neuropathic pain. Although the disclosure is not limited to one mechanism of action, MNK inhibitors as disclosed herein may disrupt peripheral sensitization.

MNKs phosphorylate the eukaryotic translation initiation factor 4E (eIF4E) and factors that bind to AU-rich elements in the 3-untranslated region of certain messenger RNAs (mRNAs). MNKs are a subfamily of Ser/Thr kinases, phylogenetically considered Ca2+/calmodulin-dependent kinases (CaMKs). MNKs are activated through phosphorylation by the growth factor-stimulated Ras/extracellular signal-regulated kinase pathway and the stress-induced p38 pathway.

Nociceptor sensitization may be blocked by inhibiting activity-dependent mRNA translation through mechanistic targeting of the mitogen-activated protein kinase (MAPK) pathway. The MAPK pathway signal to the eukaryotic translation initiation factor (eIF) 4E complex to regulate the sensitization of nociceptors.

Chemical compounds can form one or more different pharmaceutically acceptable salts and/or solid forms, including amorphous and polymorphic crystal forms. Individual salts and solid forms of bioactive chemical compounds can have different properties. There is a need for the identification and selection of appropriate salts and/or solid forms of bioactive chemical compounds (including appropriate crystalline forms, where applicable) for the development of pharmaceutically acceptable dosage forms for the treatment of various diseases or conditions associated with MNK.

### BRIEF SUMMARY

In brief, embodiments of the present disclosure provide compounds, including pharmaceutically acceptable salts, solvates, co-crystals, polymorphs, and other solid forms thereof, which are capable of inhibiting the activity of MNK.

In some embodiments, the present disclosure provides solid forms, including free base (or “free form”) solid forms, salt forms, and/or solvate forms of a compound of Structure (I).

In one aspect, the disclosure provides a solid form of a compound having the following Structure (I):

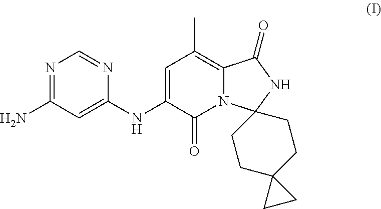 [SMILES: Cc1cc(Nc2cc(N)ncn2)c(=O)n2c1C(=O)NC21CCC2(CC2)CC1]

or a tautomer thereof, wherein the solid form has an X-ray powder diffraction pattern with at least two peaks at 2-theta angles selected from the group consisting of 5.6±0.2°, 10.9±0.2°, 18.2±0.2°, and 18.6±0.2°.

In another aspect, pharmaceutical compositions comprising the disclosed solid forms, and methods of use of the same for treatment of, e.g., inflammation, neuropathic pain, migraine pain, Lupus, viral infection-induced pain, COVID-19 related acute respiratory distress syndrome (ARDS), nonalcoholic fatty liver disease (NAFLD), high fat diet induced obesity, Alzheimer's disease, or Fragile X syndrome are also provided.

### BRIEF DESCRIPTION OF THE DRAWINGS

The patterns below refer to solid forms of Structure (I) and are a free form thereof unless indicated otherwise.

FIG. 1 shows an XRPD diffractogram of Pattern 3.

FIG. 2 shows TGA (top) and DSC (bottom) data for Pattern 3.

FIG. 3 shows an XRPD diffractogram of Pattern 11.

FIG. 4 shows TGA (top) and DSC (bottom) data for Pattern 11.

FIG. 5 shows an overlay of XRPD diffractograms for Pattern 11 and 3.

FIG. 6 shows a form diagram depicting conditions used for obtaining the 11 different polymorph forms of Structure (I) free base.

FIG. 7 is an X-ray powder diffractogram of Structure (I) from Lot A.

FIG. 8 shows TGA (top) and DSC (bottom) for Structure (I) from Lot A.

FIG. 9 is an overlay of XRPD diffractograms of different patterns of salts of Structure (I) and a free form of Structure (I).

FIG. 10 depicts an overlay of XRPD diffractograms of (from bottom to top) fumarate salt Pattern 2 that are damp, dry, and post-storage at 40° C./75% RH for 7 days.

FIG. 11 shows an XRPD diffractogram overlay of (from bottom to top), free form Pattern 3, a sulfonate salt Pattern 1, a sulfonate salt Pattern 2 before storage, and a sulfonate salt Pattern 2 after storage. Storage conditions were 40° C./75% RH for 7 days.

FIG. 12 illustrates an XRPD diffractogram overlay comparing Pattern 1 before (top) and after (bottom) a double cycle GVS experiment.

FIG. 13 shows an XRPD diffractogram overlay of Pattern 1 (bottom to top) as a reference material, after storage at 25° C./97% RH for 8 days, and storage at 40° C./75% RH for 8 days.

FIG. 14 is an XRPD diffractogram overlay of (from bottom to top) free form Pattern 3 (from Lot B), free form Pattern 2, and free form Pattern 1 (from Lot A).

FIG. 15 depicts an XRPD diffractogram overlay of free form Pattern 3 before GVS (bottom) and after GVS (top).

FIG. 16 shows an XRPD diffractogram overlay of free form Pattern 3 (bottom), free form Pattern 3 after storage at 25° C./97% RH for 7 days (middle), and after 40° C./75% RH for 7 days (top).

FIG. 17 shows an XRPD diffractogram overlay of free form Pattern 3 under various vacuum and temperature conditions. From bottom to top, conditions were 25° C.+ vacuum released after 30 minutes, 50° C. vacuum for 3 days, 50° C. vacuum, 50° C. vacuum, 50° C. vacuum, 50° C. no vacuum, and 25° C. no vacuum.

FIG. 18 shows an XRPD diffractogram overlay of Pattern 3 under various temperature conditions. From bottom to top the temperature conditions were 25° C., 250° C., 175° C., 50° C., and 25° C.

FIG. 19 is an XRPD diffractogram overlay of 5 patterns obtained from a salt screening using a 9:1 mixture of THF:water for various salts. The solid forms from bottom to top are free form Pattern 1, tartrate salt Pattern 1, fumarate salt Pattern 1, citrate salt Pattern 1, malic acid salt Pattern 1, and a succinic acid salt Pattern 1.

FIG. 20 depicts an XRPD diffractogram overlay of solids obtained from a phosphoric acid addition in a high temperature salt screening. From bottom to top, the diffractograms are phosphate salt Pattern 1 (reference), a phosphate salt Pattern 1 showing extra peaks, phosphate salt Pattern 1, a phosphate salt Pattern 2, and a phosphate salt Pattern 1 with extra peaks.

FIG. 21 shows an XRPD diffractogram overlay of poorly crystalline free form Pattern 1 (bottom) compared to free form Pattern 2 (top).

FIG. 22 shows an XRPD diffractogram overlay of (from bottom to top) free form pattern 2 (bottom), free form Pattern 1, an HBr salt Pattern 1, and an HBr salt Pattern 2 (top).

FIG. 23 shows an XRPD diffractogram overlay of (from bottom to top) an overlay of free form Pattern 1 (bottom), HCl salt Pattern 1, HCl salt Pattern 2, HCl salt Pattern 3, a combination of HCl salt Patterns 1 and 3, and HCl salt Pattern 4 (top).

FIG. 24 is an XRPD diffractogram overlay of (from bottom to top) free form Pattern 2 (bottom), free form Pattern 1, sulfate salt Pattern 1, and sulfate salt Pattern 1 (top).

FIG. 25 depicts an XRPD diffractogram overlay of (from bottom to top) free form Pattern 2 (bottom), free form Pattern 1, para-toluenesulfonic acid salt Pattern 1, and para-toluenesulfonic acid salt Pattern 1 (top).

FIG. 26 shows an XRPD diffractogram overlay of (from bottom to top) free form Pattern 2 (bottom), free form Pattern 1, methanesulfonic acid salt Pattern 1, and methanesulfonic acid salt Pattern 2 (top).

FIG. 27 shows an XRPD diffractogram overlay of (from bottom to top) free form Pattern 2, free form Pattern 1, benzenefulconic acid salt Pattern 1, and benzenesulfonic acid salt Pattern 1 (top).

FIG. 28 shows an XRPD diffractogram overlay of (from bottom to top) free form Pattern 2 (bottom), free form Pattern 1, maleic acid salt Pattern 1, and maleic acid salt Pattern 1 (top).

FIG. 29 is an XRPD diffractogram overlay of (from bottom to top) H₃PO₄ salt Pattern 1 (top), H₃PO₄ salt Pattern 2 and H₃PO₄ salt Pattern 1 (top).

FIG. 30 depicts an XRPD diffractogram overlay of (from bottom to top) free form Pattern 2 (bottom), free form Pattern 1, L-tartaric acid salt Pattern 1, and poorly crystalline material.

FIG. 31 shows an XRPD diffractogram overlay of (from bottom to top) free form Pattern 2, free form Pattern 1, fumaric acid salt Pattern 1, and poorly crystalline fumaric acid salt Pattern 1.

FIG. 32 illustrates an XRPD diffractogram overlay of (from bottom to top) free form Pattern 2 (bottom), free form Pattern 1, citric acid salt Pattern 1, and citric acid salt Pattern 1 (top).

FIG. 33 shows an XRPD diffractogram overlay of (from bottom to top) free form Pattern 2 (bottom), free form Pattern 1, L-malic acid salt Pattern 1, and L-malic acid salt Pattern 2 (top).

FIG. 34 is an XRPD diffractogram overlay of (from bottom to top) free form Pattern 2 (bottom), free form Pattern 1, succinic acid salt Pattern 1 (reassigned as free form Pattern 2), and succinic acid salt Pattern 2 (top-showing some extra peaks).

FIG. 35 depicts an XRPD diffractogram overlay of HBr salt Pattern 1 (bottom) and free form Pattern 1 (from Lot A) (top).

FIG. 36 shows a DSC thermogram of HBr salt Pattern 1.

FIG. 37 shows an XRPD diffractogram overlay of HBr salt Pattern 1 after 7 days at 40° C. and 75% relative humidity (bottom) with a reference trace for HBr salt Pattern 1 (top).

FIG. 38 shows an XRPD diffractogram overlay of (from bottom to top) HCl salt Pattern 1 (bottom), HCl salt Pattern 2, HCl salt Pattern 3, and free form Pattern 1 (from Lot A). These materials were obtained using the THF:water in a 9:1 ratio screening process as described herein below.

FIG. 39 is an XRPD diffractogram overlay of (from bottom to top) free form Pattern 1 (from Lot A), HCl salt Pattern 1 when stored for 7 days at 40° C. and 70% relative humidity, and HCl salt Pattern 1 (top).

FIG. 40 depicts an XRPD diffractogram overlay of (from bottom to top) of sulfate salt Pattern 1 (bottom) and free form Pattern 1 (from Lot A). This material was obtained using the THF:water in a 9:1 ratio screening process as described herein below.

FIG. 41 shows an XRPD diffractogram overlay of (from bottom to top) of sulfate salt Pattern 1 (bottom) and sulfate salt Pattern 1 when stored for 7 days at 40° C. and 75% relative humidity.

FIG. 42 shows an XRPD diffractogram overlay of (from bottom to top) of tosylate salt Pattern 1 and free form Pattern 1 (from Lot A).

FIG. 43 shows an XRPD diffractogram overlay of (from bottom to top) of free form Pattern 1 and free form Pattern 3 (top).

FIG. 44 is an XRPD diffractogram overlay of (from bottom to top) of besylate salt Pattern 1 and free form Pattern 1 (from Lot A).

FIG. 45 depicts an XRPD diffractogram overlay of (from bottom to top) of besylate salt Pattern 1 after 7 days at 40° C. and 70% relative humidity and besylate salt Pattern 1.

FIG. 46 shows an XRPD overlay of (from bottom to top) of maleate salt Pattern 1 and free form Pattern 1 (from Lot A).

FIG. 47 illustrates an XRPD diffractogram overlay of (from bottom to top) of maleate salt Pattern 1 after 7 days at 40° C. and 70% relative humidity and maleate salt Pattern 1.

FIG. 48 shows an XRPD diffractogram overlay of (from bottom to top) of phosphate salt Pattern 1 and free form Pattern 1 (from Lot A).

FIG. 49 is an XRPD diffractogram overlay of (from bottom to top) of phosphate salt Pattern 1 after 7 days at 40° C. and 70% relative humidity and phosphate salt Pattern 1.

FIG. 50 depicts an XRPD diffractogram overlay of (from bottom to top) of free form Pattern 1 (bottom), citric acid salt Pattern 1, and tartrate salt Pattern 1.

FIG. 51 shows an XRPD diffractogram overlay of (from bottom to top) of tartrate salt Pattern 1 after 7 days at 40° C. and 70% relative humidity and tartrate salt Pattern 1.

FIG. 52 shows an XRPD diffractogram overlay of (from bottom to top) of fumarate salt Pattern 1 and free form Pattern 1 (from Lot A).

FIG. 53 shows an XRPD diffractogram overlay of (from bottom to top) of fumarate salt Pattern 1 after 7 days at 40° C. and 70% relative humidity and fumarate salt Pattern 1.

FIG. 54 is an XRPD diffractogram overlay of (from bottom to top) of free form Pattern 1, citrate salt Pattern 1, and tartrate salt Pattern 1.

FIG. 55 depicts an XRPD diffractogram overlay of (from bottom to top) of citrate salt Pattern 1 after 7 days at 40° C. and 70% relative humidity and citrate salt Pattern 1.

FIG. 56 shows an XRPD diffractogram overlay of (from bottom to top) of poorly crystalline free form Pattern 1 and free form Pattern 2.

FIG. 57 illustrates an XRPD diffractogram overlay of (from bottom to top) of free form Pattern 1 (top), HCl salt Pattern 1, HCl salt Pattern 2, HCl salt Pattern 3, a combination of HCl salt Patterns 1 and 3, and HCl salt Pattern 4.

FIG. 58 shows an XRPD diffractogram overlay of (from bottom to top) of HCl salt Pattern 4 after 7 days at 40° C. and 70% relative humidity and HCl salt Pattern 4.

FIG. 59 is an XRPD diffractogram overlay of (from bottom to top) of free form Pattern 2, free form Pattern 1, mesylate salt Pattern 1, and mesylate salt Pattern 2.

FIG. 60 depicts an XRPD diffractogram overlay of (from bottom to top) of free form Pattern 2, free form Pattern 1, L-malate salt Pattern 1, and L-malate salt Pattern 2.

FIG. 61 shows an XRPD diffractogram overlay of (from bottom to top) of free form Pattern 3 (input), HCl salt Pattern 1 (01), phosphate salt Pattern 1 (02), phosphate salt Pattern 1 (03), fumarate salt Pattern 2 (04), fumarate salt Pattern 2 (05), mesylate salt Pattern 2 (06), and mesylate salt Pattern 2 (07).

FIG. 62 shows an XRPD diffractogram overlay of (from bottom to top) of free form Pattern 3 (input material), free form Pattern 2, HCl salt Pattern 1, HCl salt Pattern 2, HCl salt Pattern 3, HCl salt Pattern 4, and HCl salt of Pattern 1.

FIG. 63 shows an XRPD diffractogram for HCl salt Pattern 1.

FIG. 64 is an XRPD diffractogram overlay of (from bottom to top) of free form Pattern 3 (input material), free form Pattern 2, phosphate salt Pattern 1, phosphate salt Pattern 2, phosphate salt Pattern 1, and phosphate salt Pattern 1.

FIG. 65 depicts an XRPD diffractogram for phosphate salt Pattern 1.

FIG. 66 shows an XRPD diffractogram overlay of (from bottom to top) of free form Pattern 3 (input material), free form Pattern 2, fumarate salt Pattern 1, fumarate salt Pattern 2, and fumarate salt Pattern 2 (top).

FIG. 67 shows an XRPD diffractogram for fumarate salt Pattern 2.

FIG. 68 shows an XRPD diffractogram overlay of (from bottom to top) of free form Pattern 3 (input material), free form Pattern 2, mesylate salt Pattern 1, mesylate salt Pattern 2, mesylate salt Pattern 2, and mesylate salt Pattern 2.

FIG. 69 is an XRPD diffractogram for mesylate salt Pattern 2.

FIG. 70 depicts an XRPD diffractogram overlay of (from bottom to top) of free form Pattern 3, sulfate salt Pattern 1, and HCl salt Pattern 1.

FIG. 71 shows an XRPD diffractogram overlay of (from bottom to top) of Pattern 1 reference material, a sample treated with a reverse anti-solvent of DMSO/water, a sample treated with dry grinding for 30 minutes, a sample treated with a reverse anti-solvent of DMSO/TBME (Pattern 4) and free form Pattern 3 (from Lot B).

FIG. 72 shows an XRPD diffractogram overlay of (from bottom to top) of Pattern 4 after 7 days at 40° C. and 70% relative humidity, Pattern 4 before storage, and free form Pattern 3.

FIG. 73 shows an XRPD diffractogram overlay of (from bottom to top) of amorphous material after 7 days at 40° C. and 70% relative humidity, amorphous material before storage, and free form Pattern 3.

FIG. 74 is an XRPD diffractogram overlay showing diffractograms from samples prepared to explore the results of the polymorphism screening with poorly crystalline Pattern 3 described herein (polymorph screen 1). Namely, the diffractograms are (from bottom to top) free form Pattern 1, free form Pattern 2, free form Pattern 3, input material, free form Pattern 3, Pattern 5, a mixture of Pattern 3 and Pattern 5, Pattern 6, free form Pattern 3, Pattern 7, Pattern 3 (with an extra peak at) 8.3°, Pattern 8, and free form Pattern 3.

FIG. 75 depicts an XRPD diffractogram overlay showing diffractograms from samples prepared to explore the results of the polymorphism screening with poorly crystalline Pattern 3 described herein. Namely, the diffractograms are (from bottom to top) free form Pattern 1, free form Pattern 2, free form Pattern 3, input material, free form Pattern 3, free form Pattern 3, Pattern 9, free form Pattern 3, free form Pattern 3, Pattern 8, free form Pattern 3, free form Pattern 3, and free form Pattern 3.

FIG. 76 shows an XRPD diffractogram overlay showing diffractograms from samples prepared to explore the results of the polymorphism screening with amorphous material described herein (polymorph screen 2). Namely, the diffractograms are (from bottom to top) free form Pattern 1, free form Pattern 2, free form Pattern 3, amorphous input material, free form Pattern 3, free form Pattern 3, free form Pattern 3, free form Pattern 3, free form Pattern 3, Pattern 7, free form Pattern 3, Pattern 7, and free form Pattern 3.

FIG. 77 illustrates an XRPD diffractogram overlay showing diffractograms from samples prepared to explore the results of the polymorphism screening with amorphous material described herein (polymorph screen 2). Namely, the diffractograms are (from bottom to top) free form Pattern 1, free form Pattern 2, free form Pattern 3, amorphous input material, free form Pattern 3, free form Pattern 3, Pattern 8, free form Pattern 3, free form Pattern 3, Pattern 8, free form Pattern 3, free form Pattern 3, and free form Pattern 3.

FIG. 78 shows an XRPD diffractogram overlay of (from bottom to top) free form Pattern 3, Pattern 6 (wet), and Pattern 6 (dry).

FIG. 79 is an XRPD diffractogram overlay of (from bottom to top) free form Pattern 3, Pattern 7 (wet), and Pattern 7 (dry).

FIG. 80 depicts an XRPD diffractogram overlay of (from bottom to top) free form Pattern 3, Pattern 8 (wet), and Pattern 8 (dry).

FIG. 81 shows an XRPD diffractogram overlay of (from bottom to top) free form Pattern 3, Pattern 9 (wet), and Pattern 9 (dry).

FIG. 82 illustrates an XRPD diffractogram overlay showing formation of Pattern 7 (top) from slurring Pattern 3 (bottom) in methylethylketone.

FIG. 83 shows an overlay of XRPD diffractograms showing formation of Pattern 11. From bottom to top, the diffractograms are Pattern 11 (after 1 day drying at ambient temperature), Pattern 11 (after drying 1 hour at 250° C.), Pattern 10 and 11 as a reference, and Pattern 3 (from Lot B).

FIG. 84 is a readout of the thermal analysis (TGA (top) and DSC (bottom)) of Pattern 11.

FIG. 85 depicts an XRPD diffractogram overlay of (from bottom to top) of Pattern 11 after 7 days at 40° C. and 70% relative humidity, Pattern 11 after 3 days at 40° C. and 70% relative humidity, and Pattern 11 before storage.

FIG. 86 shows an overlay of XRPD diffractograms of two preparations of Pattern 11.

FIG. 87 illustrates an overlay of XRPD diffractograms of (bottom to top) Pattern 11 after GVS and Pattern 11 before GVS.

FIG. 88 shows an XRPD diffractogram overlay of (from bottom to top) Pattern 11, Pattern 11 after 10 days at 40° C. and 70% relative humidity, and Pattern 10 after 10 days at 25° C. and 97% relative humidity.

FIG. 89 is a ball and stick diagram of free form Pattern 3 as a hemihydrate.

FIG. 90 depicts a view of free form Pattern 3 as a hemihydrate from a single crystal structure showing the atom numbering scheme. Anisotropic atomic displacement ellipsoids for the non-hydrogen atoms are shown at the 50% probability level.

FIG. 91 shows a hydrogen bonding network of free form Pattern 3 (intermolecular hydrogen bonds are depicted as dashed lines).

FIG. 92 illustrates a hydrogen bonding network of free form Pattern 3 (intermolecular hydrogen bonds are depicted as dashed lines).

FIG. 93 shows crystal packing of free form Pattern 3 viewed down the crystallographic a-axis. For clarity, all hydrogen atoms have been removed from packing diagrams.

FIG. 94 is a crystal packing diagram of free form Pattern 3 viewed down the crystallographic b-axis. For clarity, all hydrogen atoms have been removed from packing diagrams.

FIG. 95 depicts a crystal packing diagram of free form Pattern 3 viewed down the crystallographic c-axis. For clarity, all hydrogen atoms have been removed from packing diagrams.

FIG. 96 shows a simulated XRPD diffractogram for free form Pattern 3 at 293 K.

FIG. 97 illustrates a comparison between an experimental diffractogram for free form Pattern 3 collected at room temperature (bottom) and the pattern simulated from the single crystal data at 293 K (top). The patterns are consistent, which confirms that the single crystal used for the structure determination is representative of the reference material. Slight differences in the simulated and experimental diffractograms are attributable to preferred orientation.

FIG. 98 is an exemplary XRPD spectrum of material containing Structure (I) Pattern 3 and additional peaks.

FIG. 99 is an overlay of a Pattern 3 reference XRPD spectrum and material from Part-2 of Example 22 after slurrying in water for 8 h.

### DETAILED DESCRIPTION

The particulars described herein are by way of example and are only for purposes of illustrative discussion of embodiments of the present disclosure. The use of any and all examples, or exemplary language (e.g., “such as” or “for example”) provided herein is merely intended to better illuminate the disclosure and does not pose a limitation on the scope of the disclosure as claimed. No language in the specification should be construed as indicating any non-claimed element is essential to the practice of the disclosure. Further, all methods described herein can be performed in any suitable order unless otherwise indicated herein or otherwise clearly contradicted by context.

The use of the alternative (e.g., “or”) should be understood to mean one, both, or any combination thereof of the alternatives. The various embodiments described above can be combined to provide further embodiments. Groupings of alternative elements or embodiments of the disclosure described herein should not be construed as limitations. Each member of a group may be referred to and claimed individually, or in any combination with other members of the group or other elements found herein.

Each embodiment disclosed herein can comprise, consist essentially of, or consist of a particular stated element, step, ingredient, or component. As used herein, the term “comprise” or “comprises” means “includes, but is not limited to,” and allows for the inclusion of unspecified elements, steps, ingredients, or components, even in major amounts. As used herein, the phrase “consisting of” excludes any element, step, ingredient, or component that is not specified. As used herein, the phrase “consisting essentially of” limits the scope of the embodiment to the specified elements, steps, ingredients, or components, and to those that do not materially affect the basic and novel characteristics of the claimed disclosure.

The terms “a,” “an,” “the,” and similar articles or terms used in the context of describing the disclosure (especially in the context of the following claims) are to be construed to cover both the singular and the plural (i.e., “one or more”), unless otherwise indicated herein or clearly contradicted by context. Ranges of values recited herein are intended to serve as a shorthand method of referring individually to each separate value falling within the range. In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as size or thickness, are to be understood to include any integer within the recited range, unless otherwise indicated. Unless otherwise indicated herein, each individual value is incorporated into the specification as if it were individually recited herein.

The term “about” has the meaning reasonably ascribed to it by a person of ordinary skill in the art when used in conjunction with a stated numerical value or range, i.e., denoting somewhat more or somewhat less than the stated value or range, to within a range of +20% of the stated value; +19% of the stated value; +18% of the stated value; +17% of the stated value; +16% of the stated value; +15% of the stated value; +14% of the stated value; =13% of the stated value; +12% of the stated value; +11% of the stated value; +10% of the stated value; +9% of the stated value; +8% of the stated value; +7% of the stated value; +6% of the stated value; =5% of the stated value; +4% of the stated value; +3% of the stated value; +2% of the stated value; or +1% of the stated value.

### Structure (I)

The compound 6″-((6-aminopyrimidin-4-yl)amino)-8″-methyl-2″H-dispiro[cyclopropane-1, l′-cyclohexane-4′,3″-imidazo[1,5-a]pyridine]-1″,5″-dione:

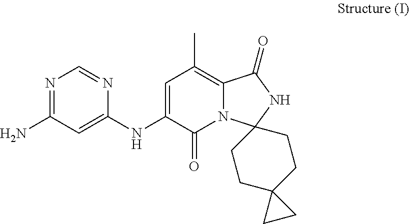 [SMILES: Cc1cc(Nc2cc(N)ncn2)c(=O)n2c1C(=O)NC21CCC2(CC2)CC1]

is an inhibitor of MNK and is further described in WO 2023/278686. Structure (I) has shown potency against MNK in various assays (see, e.g., WO 2023/278686). Accordingly, Structure (I) is useful for treating diseases, disorders, or conditions associated with MNK, such as neuropathic pain.

The present disclosure provides various free form and salt forms of Structure (I), solid forms thereof, and pharmaceutical compositions comprising the same. Salt forms and solids forms (e.g., crystalline solid forms) impart or may impart characteristics such as improved aqueous solubility, stability, hygroscopicity (e.g., provided forms may be less hygroscopic than another form), absorption, bioavailability, and ease of formulation.

It will be appreciated that a crystalline solid form of Structure (I) or a salt thereof may exist in a neat (i.e., unsolvated) form, a hydrated form, a solvated form, and/or a heterosolvated form. In some embodiments, a crystalline solid form of Structure (I) or a salt thereof does not have any water or other solvent incorporated into the crystal lattice (i.e., is “unsolvated” or an “anhydrate”). In some embodiments, a crystalline solid form of Structure (I) or a salt thereof comprises water and/or other solvent in the crystal lattice (i.e., are hydrates and/or solvates, respectively). It will be appreciated that solvates comprising only certain solvents (most notably, water) are suitable for development as a drug. Solvates comprising other solvents may be useful for manufacturing and/or testing, inter alia, even if they may not be acceptable for use in an approved therapeutic product.

Without wishing to be bound by any particular theory, the present disclosure recognizes certain challenges in obtaining Structure (I) Pattern 3 substantially free of other forms and/or impurities in a consistent manner, as well as provides a solution to this problem. As described in Example 22, slurrying material having an increased water content (e.g., greater than 3% w/w, greater than 3.5% w/w, greater than 4% w/w, or greater than 4.5% w/w) and comprising Structure (I) Pattern 3 and one or more other forms in water surprisingly resulted in Structure (I) Pattern 3 without other forms and with a lower water content (e.g., a water content corresponding to a hemihydrate form of Structure (I), such as less than 3% w/w, less than 2.8% w/w, less than 2.6% w/w, or less than 2.4% w/w).

### Free Base Pattern 3

In some embodiments, the present disclosure provides a solid form of Structure (I) referred to herein as Pattern 3. In some embodiments, Pattern 3 is a hydrate (e.g., a hemihydrate).

In particular, one embodiment provides a solid form of a compound having the following Structure (I):

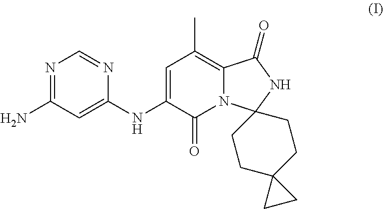 [SMILES: Cc1cc(Nc2cc(N)ncn2)c(=O)n2c1C(=O)NC21CCC2(CC2)CC1]

or a tautomer thereof, wherein the solid form has an X-ray powder diffraction pattern with at least two peaks at 2-theta angles selected from the group consisting of 5.6±0.2°, 10.9±0.2°, 18.2=0.2°, and 18.6±0.2°.

In some embodiments, the solid form has an X-ray powder diffraction pattern with at least three peaks at 2-theta angles selected from the group consisting of 5.6±0.2°, 10.9±0.2°, 18.2±0.2°, and 18.6±0.2°. In certain embodiments, the solid form has an X-ray powder diffraction pattern with peaks at 2-theta angles at 5.6±0.2°, 10.9±0.2°, 18.2±0.2°, and 18.6±0.2°. In some specific embodiments, the solid form has an X-ray powder diffraction pattern with at least two peaks at 2-theta angles selected from the group consisting of 5.6°, 10.9°, 18.2°, and 18.6°.

In certain specific embodiments, the solid form has an X-ray powder diffraction pattern with at least three peaks at 2-theta angles selected from the group consisting of 5.6°, 10.9°, 18.2°, and 18.6°. In some embodiments, the solid form has an X-ray powder diffraction pattern with peaks at 2-theta angles at 5.6°, 10.9°, 18.2°, and 18.6°.

In some embodiments, the solid form has an X-ray power diffraction pattern with peaks at 2-theta angles at 5.6±0.2°, 8.0±0.2° (e.g., 7.8°, 7.9°, 8.0°, 8.1°, or 8.2°), 8.4±0.2° (e.g., 8.2°, 8.3°, 8.4°, 8.5°, or 8.6°), 9.2±0.2°, 10.9±0.2°, 11.2±0.2°, 13.2±0.2°, 14.3±0.2°, 15.3±0.2°, 16.2±0.2° (e.g., 16.0°, 16.1°, 16.2°, 16.3°, or 16.4°), 16.5±0.2° (e.g., 16.3°, 16.4°, 16.5°, 16.6°, or 16.7°), 16.9±0.2° (e.g., 16.7°, 16.8°, 16.9°, 17.0°, or 17.1°), 17.4±0.2°, 18.2±0.2° (e.g., 18.0°, 18.1°, 18.2°, 18.3°, or 18.4°), 18.6±0.2° (e.g., 18.4°, 18.5°, 18.6°, 18.7°, or 18.8°), 19.9=0.2° (e.g., 19.7°, 19.8°, 19.9°, 20.0°, or 20.1°), 20.2±0.2° (e.g., 20.0°, 20.1°, 20.2°, 20.3°, or) 20.4°, 20.5±0.2° (e.g., 20.3°, 20.4°, 20.5°, 20.6°, or 20.7°), 21.9±0.2°, 22.3±0.2° (e.g., 22.1°, 22.2°, 22.3°, 22.4°, or 22.5°), 22.5±0.2° (e.g., 22.3°, 22.4°, 22.5°, 22.6°, or 22.7°), 23.3±0.2°, 23.6±0.2° (e.g., 23.4°, 23.5°, 23.6°, 23.7°, or 23.8°), 24.7±0.2°, 25.2±0.2°, 25.8±0.2°, 26.2±0.2°, 27.0±0.2° (e.g., 26.8°, 26.9°, 27.0°, 27.1°, or 27.2°), 27.3±0.2° (e.g., 27.1°, 27.2°, 27.3°, 27.4°, or 27.5°), 27.8±0.2°, 28.5±0.2° (e.g., 28.3°, 28.4°, 28.5°, 28.6°, or 28.7°), and 28.8±0.2° (e.g., 28.6°, 28.7°, 28.8°, 28.9°, or 29.0°).

In certain embodiments, the solid form has an X-ray powder diffraction pattern with peaks at 2-theta angles at 5.6°, 8.0°, 8.4°, 9.2°, 10.9°, 11.2°, 13.2°, 14.3°, 15.3°, 16.2°, 16.5°, 16.9°, 17.4°, 18.2°, 18.6°, 19.9°, 20.2°, 20.5°, 21.9°, 22.3°, 22.5°, 23.3°, 23.6°, 24.7°, 25.2°, 25.8°, 26.2°, 27.0°, 27.3°, 27.8°, 28.5°, and 28.8°.

In some specific embodiments, the solid form is characterized by an XRPD pattern comprising Pattern 3. In certain embodiments, the solid form is characterized by an XRPD pattern consisting essentially of Pattern 3. In some embodiments, a composition comprising the solid form is substantially pure.

One embodiment provides a solid form of a compound having the following Structure (I):

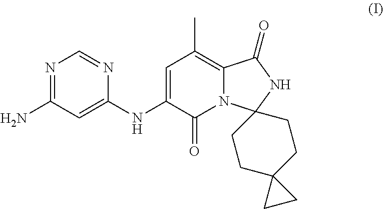 [SMILES: Cc1cc(Nc2cc(N)ncn2)c(=O)n2c1C(=O)NC21CCC2(CC2)CC1]

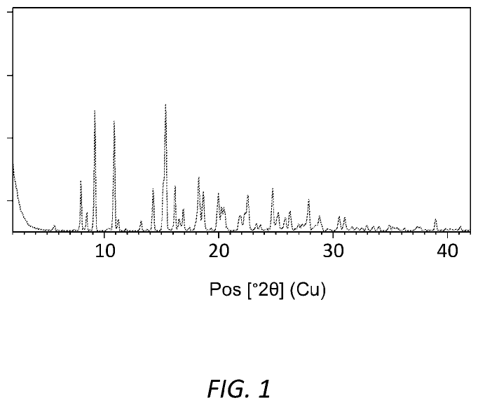

or a tautomer thereof, having an X-ray powder diffraction pattern substantially in accordance with that depicted in FIG. 1.

In some embodiments, the solid form is characterized by a differential scanning calorimetry thermogram comprising an endothermic peak with an onset of about 89.5° C. In some more specific embodiments, the endothermic peak has an area under the curve greater than 60 J/g. In some embodiments, the endothermic peak has an area under the curve greater than 65 J/g.

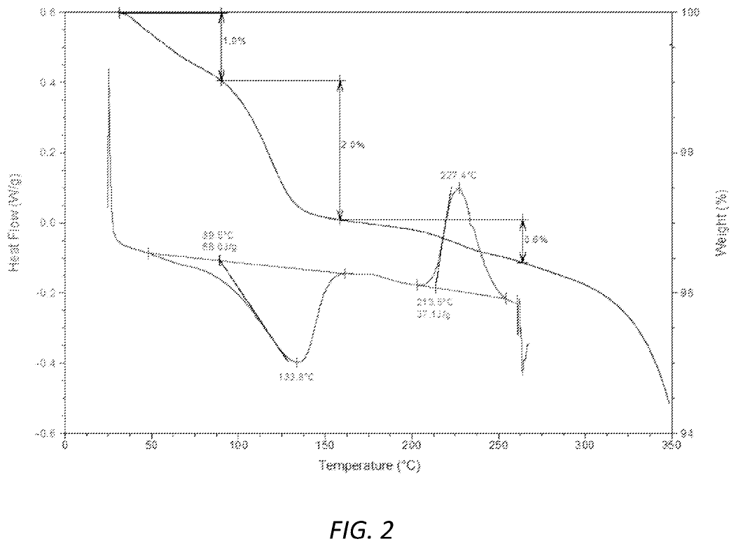

In some specific embodiments, the solid form is characterized by a differential scanning calorimetry thermogram comprising an exothermic peak with an onset of about 213.5° C. In some embodiments, the exothermic peak has an area under the curve greater than 30 J/g. In certain embodiments, the endothermic peak has an area under the curve greater than 35 J/g. In some more specific embodiments, the solid form is characterized by a differential scanning calorimetry thermogram substantially in accordance with that depicted in FIG. 2.

### Free Base Pattern 11

In some embodiments, the present disclosure provides a solid form of Structure (I) referred to herein as Pattern 11. In some embodiments, Pattern 11 is anhydrous and unsolvated.

One embodiment provides a solid form of a compound having the following Structure (I):

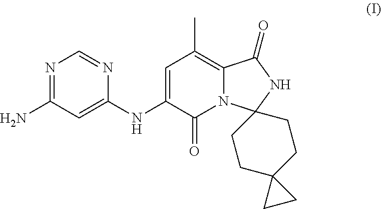 [SMILES: Cc1cc(Nc2cc(N)ncn2)c(=O)n2c1C(=O)NC21CCC2(CC2)CC1]

or a tautomer thereof, wherein the solid form has an X-ray powder diffraction pattern with at least two peaks at 2-theta angles selected from the group consisting of 19.2±0.2°, 19.5±0.2°, and 21.2±0.2°.

In some embodiments, the solid form has an X-ray powder diffraction pattern with peaks at 2-theta angles at 19.2±0.2°, 19.5±0.2°, and 21.2±0.2°. In certain embodiments, the solid form has an X-ray powder diffraction pattern with at least two peaks at 2-theta angles selected from the group consisting of 19.2°, 19.5°, and 21.2°. In some embodiments, the solid form has an X-ray powder diffraction pattern with peaks at 2-theta angles at 19.2°, 19.5°, and 21.2°.

In certain embodiments, the solid form has an X-ray powder diffraction pattern with peaks at 2-theta angles at 8.2±0.2°, 9.1±0.2°, 11.4±0.2°, 13.8±0.2°, 14.3±0.2°, 15.0±0.2°, 15.5±0.2°, 16.5±0.2°, 17.0±0.2°, 19.2±0.2° (e.g., 19.0°, 19.1°, 19.2°, 19.3°), 19.4°, 19.5±0.2° (e.g., 19.3°, 19.4°, 19.5°, 19.6°, 19.7°), 19.9±0.2° (e.g., 19.7°, 19.8°, 19.9°, 20.0°, 20.1°), 21.2±0.2°, 22.3±0.2° (e.g., 22.1°, 22.2°, 22.3°, 22.4°), 22.5°, 22.7±0.2° (e.g., 22.5°, 22.6°, 22.7°, 22.8°, 22.9°), 23.3±0.2°, 23.9±0.2°, 24.7±0.2°, 25.3±0.2°, 26.0±0.2°, 26.9±0.2°, 27.7±0.2°, 28.5±0.2°, 28.9±0.2°, and 29.7±0.2°.

In some embodiments, the solid form has an X-ray powder diffraction pattern with peaks at 2-theta angles at 8.2°, 9.1°, 11.4°, 13.8°, 14.3°, 15.0°, 15.5°, 16.5°, 17.0°, 19.2°, 19.5°, 19.9°, 21.2°, 22.3°, 22.7°, 23.3°, 23.9°, 24.7°, 25.3°, 26.0°, 26.9°, 27.7°, 28.5°, 28.9°, and 29.7°.

In some embodiments, the solid form is characterized by an XRPD pattern comprising Pattern 11. In some embodiments, the solid form is characterized by an XRPD pattern consisting essentially of Pattern 11. In certain specific embodiments, a composition comprising the solid form is substantially pure.

One embodiment provides a solid form of a compound having the following Structure (I):

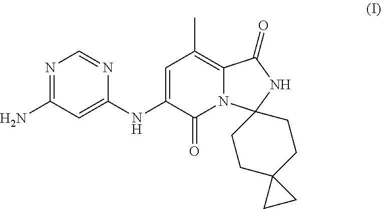 [SMILES: Cc1cc(Nc2cc(N)ncn2)c(=O)n2c1C(=O)NC21CCC2(CC2)CC1]

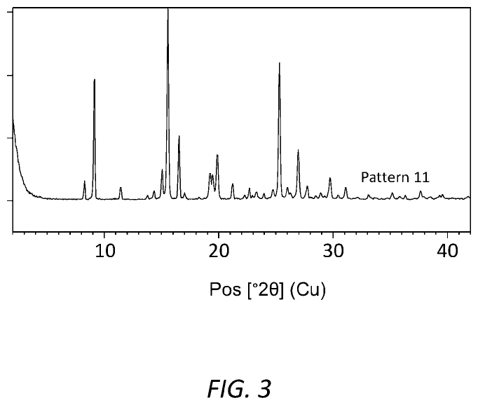

or a tautomer thereof, having an X-ray powder diffraction pattern substantially in accordance with that depicted in FIG. 3.

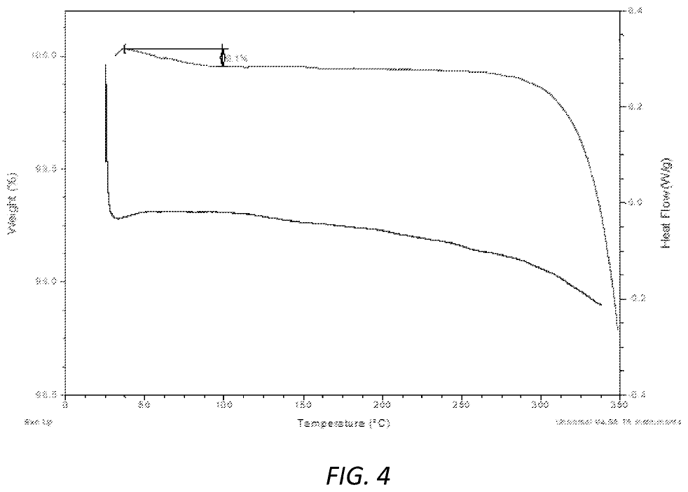

In some embodiments, the solid form is characterized by a differential scanning calorimetry thermogram comprising no events up to 340° C. In certain embodiments, the solid form is characterized by a differential scanning calorimetry thermogram substantially in accordance with that depicted in FIG. 4.

### Other Free Base Forms

In some embodiments, the present disclosure provides Structures (I) in various free base forms, including amorphous and crystalline forms.

In some embodiments, the present disclosure provides Structure (I) in a crystalline solid form. Exemplary crystalline solid forms of Structure (I), and methods of preparing the same, are described in the Examples below.

In some embodiments, the present disclosure provides Structure (I) Pattern 1. In some embodiments, the present disclosure provides Structure (I) Pattern 2. In some embodiments, the present disclosure provides Structure (I) Pattern 4. In some embodiments, the present disclosure provides Structure (I) Pattern 5. In some embodiments, the present disclosure provides Structure (I) Pattern 6. In some embodiments, the present disclosure provides Structure (I) Pattern 7. In some embodiments, the present disclosure provides Structure (I) Pattern 8. In some embodiments, the present disclosure provides Structure (I) Pattern 9. In some embodiments, the present disclosure provides Structure (I) Pattern 10.

In some embodiments, the present disclosure provides Structure (I) in an amorphous form. Exemplary amorphous forms of Structure (I), and methods of preparing the same, are described in the Examples below.

### Salt Forms of Structure (I)

In some embodiments, the present disclosure provides solid forms (i.e., salts or co-crystals) of Structure (I), wherein Structure (I) and a co-former are, e.g., ionically bonded or are hydrogen bonded to form a provided form described herein. When Structure (I) salt forms are in a solid form, they may be amorphous, crystalline, or a mixture thereof. Exemplary salt forms of Structure (I), and methods of preparing the same, are described in the Examples below.

In some embodiments, the present disclosure provides a salt form of Structure (I) formed between Structure (I) and a co-former selected from hydrochloric acid, hydrobromic acid, sulfuric acid, acetic acid, maleic acid, fumaric acid, phosphoric acid, citric acid, p-toluenesulfonic acid, methanesulfonic acid, benzenesulfonic acid, tartaric acid, succinic acid, and malic acid.

In some embodiments, the present disclosure provides a Structure (I) salt form formed between Structure (I) and hydrochloric acid. In some embodiments, a Structure (I) salt form is HCl Pattern 1. In some embodiments, a Structure (I) salt form is HCl Pattern 2. In some embodiments, a Structure (I) salt form is HCl Pattern 3. In some embodiments, a Structure (I) salt form is HCl Pattern 4.

In some embodiments, the present disclosure provides a Structure (I) salt form formed between Structure (I) and hydrobromic acid. In some embodiments, a Structure (I) salt form is HBr Pattern 1.

In some embodiments, the present disclosure provides a Structure (I) salt form formed between Structure (I) and sulfuric acid. In some embodiments, a Structure (I) salt form is Sulfate Pattern 1.

In some embodiments, the present disclosure provides a Structure (I) salt form formed between Structure (I) and maleic acid. In some embodiments, a Structure (I) salt form is Maleate Pattern 1.

In some embodiments, the present disclosure provides a Structure (I) salt form formed between Structure (I) and fumaric acid. In some embodiments, a Structure (I) salt form is Fumarate Pattern 1. In some embodiments, a Structure (I) salt form is Fumarate Pattern 2.

In some embodiments, the present disclosure provides a Structure (I) salt form formed between Structure (I) and phosphoric acid. In some embodiments, a Structure (I) salt form is Phosphate Pattern 1. In some embodiments, a Structure (I) salt form is Phosphate Pattern 2.

In some embodiments, the present disclosure provides a Structure (I) salt form formed between Structure (I) and citric acid. In some embodiments, a Structure (I) salt form is Citrate Pattern 1.

In some embodiments, the present disclosure provides a Structure (I) salt form formed between Structure (I) and p-toluenesulfonic acid. In some embodiments, a Structure (I) salt form is Tosylate Pattern 1.

In some embodiments, the present disclosure provides a Structure (I) salt form formed between Structure (I) and methanesulfonic acid. In some embodiments, a Structure (I) salt form is Mesylate Pattern 2.

In some embodiments, the present disclosure provides a Structure (I) salt form formed between Structure (I) and benzenesulfonic acid. In some embodiments, a Structure (I) salt form is Besylate Pattern 1.

In some embodiments, the present disclosure provides a Structure (I) salt form formed between Structure (I) and tartaric acid. In some embodiments, a Structure (I) salt form is Tartrate Pattern 1.

In some embodiments, the solid form comprises, consists essentially of, or consists of Pattern 1. In some embodiments, the solid form is a salt of Pattern 1. In some embodiments, the solid form is a co-crystal, solvate, or free form of Pattern 1. In certain embodiments, the solid form is a pharmaceutically acceptable salt of Pattern 1 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 2. In some embodiments, the solid form is a salt of Pattern 2. In some embodiments, the solid form is a co-crystal, solvate, or free form of Pattern 2. In certain embodiments, the solid form is a pharmaceutically acceptable salt of Pattern 2 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 3. In some embodiments, the solid form is a salt of Pattern 3. In some embodiments, the solid form is a co-crystal, solvate, or free form of Pattern 3. In certain embodiments, the solid form is a pharmaceutically acceptable salt of Pattern 3 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 4. In some embodiments, the solid form is a salt of Pattern 4. In some embodiments, the solid form is a co-crystal, solvate, or free form of Pattern 4. In certain embodiments, the solid form is a pharmaceutically acceptable salt of Pattern 4 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 5. In some embodiments, the solid form is a salt of Pattern 5. In some embodiments, the solid form is a co-crystal, solvate, or free form of Pattern 5. In certain embodiments, the solid form is a pharmaceutically acceptable salt of Pattern 5 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 6. In some embodiments, the solid form is a salt of Pattern 6. In some embodiments, the solid form is a co-crystal, solvate, or free form of Pattern 6. In certain embodiments, the solid form is a pharmaceutically acceptable salt of Pattern 6 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 7. In some embodiments, the solid form is a salt of Pattern 7. In some embodiments, the solid form is a co-crystal, solvate, or free form of Pattern 7. In certain embodiments, the solid form is a pharmaceutically acceptable salt of Pattern 7 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 8. In some embodiments, the solid form is a salt of Pattern 8. In some embodiments, the solid form is a co-crystal, solvate, or free form of Pattern 8. In certain embodiments, the solid form is a pharmaceutically acceptable salt of Pattern 8 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 9. In some embodiments, the solid form is a salt of Pattern 9. In some embodiments, the solid form is a co-crystal, solvate, or free form of Pattern 9. In certain embodiments, the solid form is a pharmaceutically acceptable salt of Pattern 9 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 10. In some embodiments, the solid form is a salt of Pattern 10. In some embodiments, the solid form is a co-crystal, solvate, or free form of Pattern 10. In certain embodiments, the solid form is a pharmaceutically acceptable salt of Pattern 10 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some embodiments, the solid form comprises, consists essentially of, or consists of Pattern 11. In some embodiments, the solid form is a salt of Pattern 11. In some embodiments, the solid form is a co-crystal, solvate, or free form of Pattern 11. In certain embodiments, the solid form is a pharmaceutically acceptable salt of Pattern 11 (e.g., a chloride salt, a bromide salt, a sulfate salt, an acetate salt, a maleate salt, a fumarate salt, a phosphate salt, a citrate salt, a tosylate salt, a mesylate salt, a besylate salt, a tartrate salt, a succinate salt, or a malate salt).

In some of the foregoing embodiments, the salt is formed from hydrobromic acid, hydrochloric acid, sulfuric acid, p-toluenesulfonic acid, methanesulfonic acid, benzene sulfonic acid, maleic acid, phosphoric acid, L-tartaric acid, fumaric acid, citric acid, L-malic acid, or succinic acid.

### Methods of Preparing Provided Forms

Solid forms (e.g., crystalline and amorphous free base and salt forms) can be prepared according to the methods described in the Examples.

In some embodiments, the present disclosure provides a method for preparing a Structure (I) form described herein, comprising one or more steps of removing a solvent or adding a solvent. In some embodiments, the added solvent is the same as the solvent removed. In some embodiments, an added solvent is different from a solvent removed. Means of solvent removal are known in the synthetic and chemical arts and include, but are not limited to, any of those described herein and in the Examples.

In some embodiments, a method for preparing a Structure (I) form described herein comprises one or more steps of heating or cooling a preparation. In some embodiments, a method for preparing a Structure (I) form described herein comprises one or more steps of agitating or stirring a preparation. In some embodiments, a method for preparing a Structure (I) form described herein comprises a step of adding a suitable co-former to a solution or slurry of Structure (I). In some embodiments, a method for preparing a Structure (I) form described herein comprises a step of adding a suitable acid to a solution or slurry of Structure (I).

In some embodiments, a Structure (I) form described herein precipitates from the mixture. In another embodiment, a Structure (I) form described herein crystallizes from the mixture.

A Structure (I) form described herein can precipitate out of the reaction mixture, or be generated by removal of part or all of the solvent through methods such as evaporation, distillation, filtration (e.g., nanofiltration, ultrafiltration), reverse osmosis, absorption and reaction, by adding a suitable anti-solvent, by cooling or by different combinations of these methods.

As described generally herein, a Structure (I) form is optionally isolated. It will be appreciated that a Structure (I) form may be isolated by any suitable physical means known to one of ordinary skill in the art. In certain embodiments, a precipitated solid Structure (I) form is separated from the supernatant by filtration. In other embodiments, a precipitated solid Structure (I) form is separated from the supernatant by decanting the supernatant.

In some embodiments, a Structure (I) form is optionally purified. It will be appreciated that a Structure (I) form may be purified by any suitable physical means known to one of ordinary skill in the art. In some embodiments, a crude Structure (I) form is slurried in a suitable solvent (e.g., water) to provide a Structure (I) form in greater purity.

### Compositions

One embodiment provides a pharmaceutical composition comprising a solid form of any one of the embodiments disclosed herein and a pharmaceutically acceptable carrier or excipient. In some embodiments, the pharmaceutical composition is formulated for oral administration. In some embodiments, the pharmaceutical composition is in the form of a capsule. In some embodiments, the pharmaceutical composition is in the form of a tablet.

In some embodiments, the pharmaceutical composition is formulated as part of an aqueous solution. In some embodiments, the pharmaceutical composition is formulated for injection. In some embodiments, the pharmaceutical composition is formulated for administration via an intravenous, intramuscular, or subcutaneous route. In some embodiments, the pharmaceutical composition is formulated for a rectal or vaginal route. In some embodiments, the pharmaceutical composition is formulated for inhalation.

In some embodiments, the present disclosure provides a composition comprising a Structure (I) form, wherein the composition is substantially free of impurities. As used herein, the term “substantially free of impurities” means that the composition contains no significant amount of extraneous matter. Such extraneous matter may include different forms of Structure (I), residual solvents, or any other impurities that may result from the preparation of, and/or isolation of, Structure (I). In certain embodiments, at least about 95% by weight of a form of Structure (I) is present. In certain embodiments, at least about 95%, about 96%, about 97%, about 98%, or about 99% by weight of a form of Structure (I) is present. In still other embodiments of the disclosure, at least about 99% by weight of a form of Structure (I) is present.

In some embodiments, the present disclosure provides a composition comprising a crystalline Structure (I) form (e.g., Pattern 3 or Pattern 11), wherein the composition is substantially free of other crystalline or amorphous forms of Structure (I). For examples, such composition contains no significant amount of the other crystalline or amorphous forms of Structure (I). In certain embodiments, at least about 95% by weight of the crystalline form of Structure (I) is present. In certain embodiments, at least about 95%, about 96%, about 97%, about 98%, or about 99% by weight of the crystalline form of Structure (I) is present. In still other embodiments of the disclosure, at least about 99% by weight of the crystalline form of Structure (I) is present.

In some embodiments, the present disclosure provides a composition comprising an amorphous Structure (I) form, wherein the composition is substantially free of crystalline forms of Structure (I). For examples, such composition contains no significant amount of crystalline forms of Structure (I). In certain embodiments, at least about 95% by weight of the amorphous form of Structure (I) is present. In certain embodiments, at least about 95%, about 96%, about 97%, about 98%, or about 99% by weight of the amorphous form of Structure (I) is present. In still other embodiments of the disclosure, at least about 99% by weight of the amorphous form of Structure (I) is present.

### Methods of Use

A particular embodiment provides a method for treating, preventing, or mitigating the effects of a migraine or symptoms related to a migraine, the method comprising administering a therapeutically effective amount of the solid form of any one of the embodiments disclosed herein, or a pharmaceutical composition thereof.

One embodiment provides a method for treating, preventing, or mitigating the effects of a disease associated with aberrant MNK activity in a mammal in need thereof, comprising administering to the mammal a therapeutically effective amount of a solid form of any one of the embodiments disclosed herein, or a pharmaceutical composition thereof.

One embodiment provides a method for treating, preventing, or mitigating the effects of neuropathic pain, Lupus, viral infection-induced pain, COVID-19 related acute respiratory distress syndrome (ARDS), nonalcoholic fatty liver disease (NAFLD), high fat diet induced obesity, Alzheimer's disease, or Fragile X syndrome in a mammal in need thereof, comprising administering to the mammal a therapeutically effective amount of a solid form of any one of the embodiments disclosed herein, or a pharmaceutical composition thereof.

### EXEMPLARY EMBODIMENTS

The following numbered embodiments, while non-limiting, are exemplary of certain aspects of the present disclosure:
1. A solid form of a compound having the following Structure (I):

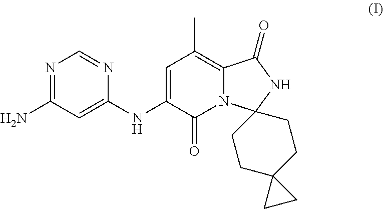 [SMILES: Cc1cc(Nc2cc(N)ncn2)c(=O)n2c1C(=O)NC21CCC2(CC2)CC1]

or a tautomer thereof, wherein the solid form has an X-ray powder diffraction pattern with at least two peaks at 2-theta angles selected from the group consisting of 5.6±0.2°, 10.9±0.2°, 18.2±0.2°, and 18.6±0.2°.
2. The solid form of embodiment 1, wherein the solid form has an X-ray powder diffraction pattern with at least three peaks at 2-theta angles selected from the group consisting of 5.6±0.2°, 10.9±0.2°, 18.2±0.2°, and 18.6±0.2°. 3. The solid form of embodiment 1, wherein the solid form has an X-ray powder diffraction pattern with peaks at 2-theta angles at 5.6±0.2°, 10.9±0.2°, 18.2±0.2°, and 18.6±0.2°.
4. The solid form of embodiment 1, wherein the solid form has an X-ray powder diffraction pattern with at least two peaks at 2-theta angles selected from the group consisting of 5.6°, 10.9°, 18.2°, and 18.6°.
5. The solid form of embodiment 1, wherein the solid form has an X-ray powder diffraction pattern with at least three peaks at 2-theta angles selected from the group consisting of 5.6°, 10.9°, 18.2°, and 18.6°.
6. The solid form of embodiment 1, wherein the solid form has an X-ray powder diffraction pattern with peaks at 2-theta angles at 5.6°, 10.9°, 18.2°, and 18.6°.
7 The solid form of embodiment 1, wherein the solid form has an X-ray powder diffraction pattern with peaks at 2-theta angles at 5.6±0.2°, 8.0±0.2°, 8.4±0.2°, 9.2±0.2°, 10.9±0.2°, 11.2±0.2°, 13.2±0.2°, 14.3±0.2°, 15.3±0.2°, 16.2±0.2°, 16.5±0.2°, 16.9±0.2°, 17.4±0.2°, 18.2±0.2°, 18.6±0.2°, 19.9±0.2°, 20.2±0.2°, 20.5±0.2°, 21.9±0.2°, 22.3±0.2°, 22.5±0.2°, 23.3±0.2°, 23.6±0.2°, 24.7±0.2°, 25.2±0.2°, 25.8±0.2°, 26.2±0.2°, 27.0±0.2°, 27.3±0.2°, 27.8±0.2°, 28.5±0.2°, and 28.8±0.2°.
8. The solid form of embodiment 1, wherein the solid form has an X-ray powder diffraction pattern with peaks at 2-theta angles at 5.6°, 8.0°, 8.4°, 9.2°, 10.9°, 11.2°, 13.2°, 14.3°, 15.3°, 16.2°, 16.5°, 16.9°, 17.4°, 18.2°, 18.6°, 19.9°, 20.2°, 20.5°, 21.9°, 22.3°, 22.5°, 23.3°, 23.6°, 24.7°, 25.2°, 25.8°, 26.2°, 27.0°, 27.3°, 27.8°, 28.5°, and 28.8°.
9 The solid form of embodiment 1, wherein the solid form comprises Pattern 3.
10. The solid form of embodiment 1, wherein the solid form consists essentially of Pattern 3.
11. The solid form of embodiment 1, wherein the solid form is substantially pure.
12. A solid form of a compound having the following Structure (I):

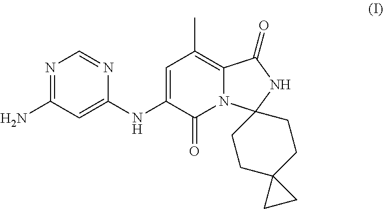 [SMILES: Cc1cc(Nc2cc(N)ncn2)c(=O)n2c1C(=O)NC21CCC2(CC2)CC1]

or a tautomer thereof, having an X-ray powder diffraction pattern substantially in accordance with that depicted in FIG. 1.
13. The solid form of any one of embodiments 1-12, characterized by a differential scanning calorimetry thermogram comprising an endothermic peak with an onset of about 89.5° C. 14. The solid form of embodiment 13, wherein the endothermic peak is greater than 60 J/g.
15. The solid form of embodiment 13, wherein the endothermic peak is greater than 65 J/g.
16. The solid form of any one of embodiments 1-15, characterized by a differential scanning calorimetry thermogram comprising an exothermic peak with an onset of about 213.5° C.
17. The solid form of embodiment 16, wherein the exothermic peak is greater than 30 J/g.
18. The solid form of embodiment 16, wherein the endothermic peak is greater than 35 J/g.
19. The solid form of any one of embodiments 1-18, characterized by a differential scanning calorimetry thermogram substantially in accordance with that depicted in FIG. 2.
20. A solid form of a compound having the following Structure (I):

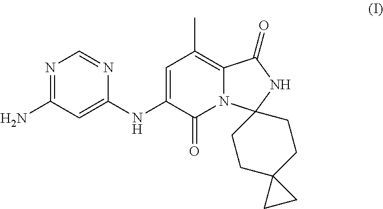 [SMILES: Cc1cc(Nc2cc(N)ncn2)c(=O)n2c1C(=O)NC21CCC2(CC2)CC1]

or a tautomer thereof, wherein the solid form has an X-ray powder diffraction pattern with at least two peaks at 2-theta angles selected from the group consisting of 19.2±0.2°, 19.5±0.2°, and 21.2±0.2°.
21. The solid form of embodiment 20, wherein the solid form has an X-ray powder diffraction pattern with peaks at 2-theta angles at 19.2±0.2°, 19.5±0.2°, and 21.2±0.2°.
22. The solid form of embodiment 20, wherein the solid form has an X-ray powder diffraction pattern with at least two peaks at 2-theta angles selected from the group consisting of 19.2°, 19.5°, and 21.2°.
23. The solid form of embodiment 20, wherein the solid form has an X-ray powder diffraction pattern with peaks at 2-theta angles at 19.2°, 19.5°, and 21.2°.
24. The solid form of embodiment 20, wherein the solid form has an X-ray powder diffraction pattern with peaks at 2-theta angles at 8.2±0.2°, 9.1±0.2°, 11.4±0.2°, 13.8±0.2°, 14.3±0.2°, 15.0±0.2°, 15.5±0.2°, 16.5±0.2°, 17.0±0.2°, 19.2±0.2°, 19.5±0.2°, 19.9±0.2°, 21.2±0.2°, 22.3±0.2°, 22.7±0.2°, 23.3±0.2°, 23.9±0.2°, 24.7±0.2°, 25.3±0.2°, 26.0±0.2°, 26.9±0.2°, 27.7±0.2°, 28.5±0.2°, 28.9±0.2°, and 29.7±0.2°.
25. The solid form of embodiment 20, wherein the solid form has an X-ray powder diffraction pattern with peaks at 2-theta angles at 8.2°, 9.1°, 11.4°, 13.8°, 14.3°, 15.0°, 15.5°, 16.5°, 17.0°, 19.2°, 19.5°, 19.9°, 21.2°, 22.3°, 22.7°, 23.3°, 23.9°, 24.7°, 25.3°, 26.0°, 26.9°, 27.7°, 28.5°, 28.9°, and 29.7°.
26. The solid form of embodiment 20, wherein the solid form comprises Pattern 11.
27. The solid form of embodiment 20, wherein the solid form consists essentially of Pattern 11.
28. The solid form of embodiment 20, wherein the solid form is substantially pure.
29. A solid form of a compound having the following Structure (I):

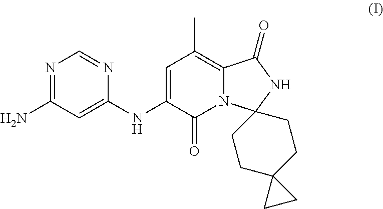 [SMILES: Cc1cc(Nc2cc(N)ncn2)c(=O)n2c1C(=O)NC21CCC2(CC2)CC1]

or a tautomer thereof, having an X-ray powder diffraction pattern substantially in accordance with that depicted in FIG. 3.
30. The solid form of any one of embodiments 20-29, characterized by a differential scanning calorimetry thermogram comprising no events until 340° C.
31. The solid form of any one of embodiments 20-29, characterized by a differential scanning calorimetry thermogram substantially in accordance with that depicted in FIG. 4.
32. A pharmaceutical composition comprising a solid form of any one of embodiments 1-31 and a pharmaceutically acceptable carrier or excipient.
33. The pharmaceutical composition of embodiment 32, formulated for oral administration.
34. The pharmaceutical composition of embodiment 32, in the form of a capsule.
35. The pharmaceutical composition of embodiment 32, in the form of a tablet.
36. A method for treating, preventing, or mitigating the effects of a migraine or symptoms related to a migraine, the method comprising administering a therapeutically effective amount of the solid form of any one of embodiments 1-31 or a pharmaceutical composition of any one of embodiments 32-35.
37. A method for treating, preventing, or mitigating the effects of a disease associated with aberrant MNK activity in a mammal in need thereof, comprising administering to the mammal a therapeutically effective amount of a solid form of any one of embodiments 1-31, or a pharmaceutical composition of any one of embodiments 32-35.
38. A method for treating, preventing, or mitigating the effects of neuropathic pain, Lupus, viral infection-induced pain, COVID-19 related acute respiratory distress syndrome (ARDS), nonalcoholic fatty liver disease (NAFLD), high fat diet induced obesity, Alzheimer's disease, or Fragile X syndrome in a mammal in need thereof, comprising administering to the mammal a therapeutically effective amount of a solid form of any one of embodiments 1-31, or a pharmaceutical composition of any one of embodiments 32-35.

### EXAMPLES

Specific conditions for preparing and obtaining the data are given below.

### Abbreviations/Initialisms/Acronyms

¹³C NMR=Carbon Nuclear Magnetic Resonance
¹H NMR=Proton Nuclear Magnetic Resonance
1-PrOH=1-Propanol
2D=2 Dimensional
2-Me-1-PrOH=2-Methyl-1-propanol
ACN=acetonitrile
ADD=Additional Peaks
API=Active Pharmaceutical Ingredient
AS=Antisolvent
ASR=Analytical Service Report
ca.=Approximately
DMSO=Dimethyl sulfoxide
D-PAS=Dip Probe Absorption Spectroscopy
DSC=Differential Scanning calorimetry
DVS=Dynamic Vapor Sorption
Eq/Eq./Equiv.=Equivalents
EtOAc=Ethyl acetate
EtOH=Ethanol
FaSSGF=Fasted state simulated gastric fluid
FaSSIF=Fasted state simulated intestinal fluid
FeSSIF=Fed state simulated intestinal fluid
GVS=Gravimetric Vapor Sorption
H₂O=Water
HBr=Hydrobromic acid
HCl=Hydrochloric acid
HPLC=High Performance Liquid Chromatography
Hr or hr=Hour
HSM=Hot Stage Microscopy
IC=Ion Chromatography
ID=Identification
IPA=2-Propanol
iPrOAc=Isopropyl acetate
IR=Infrared Spectroscopy
ISA=Ionic Strength Adjusted
KF=Karl Fischer
MALe=maleate
MALi=L-malate
MDSC=Modulated Differential Scanning calorimetry
MeCN=Acetonitrile
MEK=Methyl ethyl ketone
MeOH=Methanol
MIBK=Methyl isobutyl ketone
Min or min=Minutes
mol=Molar
MS=Mass Spectroscopy
N/A=Not Applicable
NMR=Nuclear Magnetic Resonance
No.=Number
P=Pattern
PE=Polyethylene
PLM=Polarised Light Microscopy
PTFE=Polytetrafluoroethylene
RH=Relative Humidity
RRT=Relative Retention Time
RT=Room Temperature
SCXRD=Single Crystal X-Ray Diffraction
SGF=Simulated gastric fluid
SIF=Simulated intestinal fluid
SUC=Succinate
TAR=L-Tartaric acid (or salt thereof)
TBME=tert-Butyl methyl ether
Temp=Temperature
TFA=Trifluoroacetic acid
Tg=Glass transition temperature
TGA=Thermal Gravimetric Analysis
THF=Tetrahydrofuran
TRIS=Tris(hydroxymethyl)aminomethane
USP=United States Pharmacopeia
UV=Ultraviolet
v/v=Volume to volume ratio
vac=vacuum
Vol=Volumes
w/w=Weight to weight ratio
wt=Weight
wt %=Weight %
XRPD=X-Ray Powder Diffraction

### General Methods

### X-ray Powder Diffraction (XRPD)

### Bruker AXS D8 Advance

XRPD diffractograms were collected on a Bruker D8 diffractometer using Cu Kα radiation (40 kV, 40 mA, λ=1.540562 Å) and a 0-20 goniometer fitted with a Ge monochromator. The incident beam passed through a 2.0 mm divergence slit followed by a 0.2 mm anti-scatter slit and knife edge. The diffracted beam passed through an 8.0 mm receiving slit with 2.5° Soller slits followed by the Lynxeye Detector. The software used for data collection and analysis was Diffrac Plus XRD Commander and HighScore Plus respectively.

Samples were run under ambient conditions as flat plate specimens as powder. The sample was prepared on a polished, zero-background (510) silicon wafer by gently pressing onto the flat surface or packed into a cut cavity. The sample was rotated in its own plane.

The details of the standard Pharmorphix data collection method are:

$$Angular range : 2 to 42 ° 2 θ$$

$$Step size : 0.05 ° 2 θ$$

$$Collection time : 0.5 s / step ( total collection time : 6.4 min )$$

### PANalytical Empyrean

XRPD diffractograms were collected on a PANalytical Empyrean diffractometer using Cu Kα radiation (45 kV, 40 mA, λ=1.544398 Å) in transmission geometry. A 0.5° slit, 4 mm mask and 0.04 rad Soller slits with a focusing mirror were used on the incident beam. A PIXcel3D detector, placed on the diffracted beam, was fitted with a receiving slit and 0.04 rad Soller slits. The software used for data collection was X'Pert Data Collector using X'Pert Operator Interface. The data were analyzed and presented using HighScore Plus.

Samples were prepared and analyzed in either a metal or Millipore 96 well-plate in transmission mode. X-ray transparent film was used between the metal sheets on the metal well-plate (approximately 1-2 mg). The Millipore plate was used to isolate and analyze solids from suspensions by adding a small amount of suspension directly to the plate before filtration under a light vacuum.

The scan mode for the metal plate used the gonio scan axis, whereas a 20 scan was utilized for the Millipore plate.

The details of the standard screening data collection method are:

$$Angular range : 2.5 to 32. ° 2 θ$$

$$Step size : 0.013 ° 2 θ$$

$$Collection time : 12.75 s / step ( total collection time of 2.07 min )$$

### Non-Ambient Conditions

XRPD diffractograms were collected on a PANalytical Empyrean diffractometer using Cu Kα radiation (45 kV, 40 mA, λ=1.544398 Å) in reflection geometry. The instrument was fitted with an Anton Paar CHC plus+ stage fitted with graphite/Kapton windows and equipped with air cooling coupled or a low vacuum pump system using an Edwards RV3 pump. A programmable divergence slit (in automatic mode), with a 10 mm fixed incident beam mask, Ni filter and 0.04 rad Soller slits were used on the incident beam. A PIXcel3D detector, placed on the diffracted beam, was fitted with a programmable anti-scatter slit (in automatic mode) and 0.04 rad Soller slits.

The software used for data collection was X'Pert Data Collector and the data analyzed and presented using Highscore Plus.

For variable temperature (VT-XRPD) experiments the samples were prepared and analyzed in an Anton Paar chromed sample holder. The sample chamber was under ambient atmosphere and a heating/cooling rate of 10° C./min was used with a 2 min isothermal hold before the measurement started. The measurement parameters are as per the standard screening data collection method (detailed above). Measurements were taken at the following temperatures: 25, 80, 170, 250, and 25° C.

For XRPD under vacuum the samples were prepared and analyzed in an Anton Paar chromed sample holder. The sample temperature was kept constant throughout at 25° C. The measurement parameters are as per the standard screening data collection method (detailed above). An initial measurement was taken before the vacuum was initiated and then subsequently at 10 min intervals. Data collection was stopped after three consecutive scans showing no change.

### Nuclear Magnetic Resonance (NMR)

¹H NMR and/or 13C NMR spectra were collected on a Bruker 400 MHz instrument equipped with an auto-sampler and controlled by a DRX400 console. Samples were prepared in DMSO-d₆ solvent, unless otherwise stated. Automated experiments were acquired using ICON-NMR configuration within Topspin software, using standard Bruker-loaded experiments (¹H, ¹³C {¹H}, DEPT135). Off-line analysis was performed using ACD Spectrus Processor.

For non-routine spectroscopy (2D NMR and variable temperature NMR), data were acquired through the use of Topspin alone.

### Differential Scanning Calorimetry (DSC)

### TA Instruments Q2000

DSC data were collected on a TA Instruments Q2000 equipped with a 50 position auto-sampler. Typically, 0.5-3 mg of each sample, in a pin-holed aluminum pan, was heated at 10° C./min from 25° C. to 300° C. A purge of dry nitrogen at 50 mL/min was maintained over the sample.

The instrument control software was Advantage for Q Series and Thermal Advantage and the data were analyzed using Universal Analysis or TRIOS.

### TA Instruments Discovery DSC

DSC data were collected on a TA Instruments Discovery DSC equipped with a 50 position auto-sampler. Typically, 0.5-3 mg of each sample, in a pin-holed aluminum pan, was heated at 10° C./min from 25° C. to 300° C. A purge of dry nitrogen at 50 mL/min was maintained over the sample.

The instrument control software was TRIOS and the data were analyzed using TRIOS or Universal Analysis.

### Thermo-Gravimetric Analysis (TGA)

### TA Instruments Q500

TGA data were collected on a TA Instruments Q500 TGA, equipped with a 16 position auto-sampler. Typically, 5-10 mg of each sample was loaded onto a pre-tared aluminum DSC pan and heated at 10° C./min from ambient temperature to 350° C. A nitrogen purge at 60 mL/min was maintained over the sample.

The instrument control software was Advantage for Q Series and Thermal Advantage and the data were analyzed using Universal Analysis or TRIOS.

### TA Instruments Discovery TGA

TGA data were collected on a TA Instruments Discovery TGA, equipped with a 25-position auto-sampler. Typically, 5-10 mg of each sample was loaded onto a pre-tared aluminum DSC pan and heated at 10° C./min from ambient temperature to 350° C. A nitrogen purge at 25 mL/min was maintained over the sample.

The instrument control software was TRIOS and the data were analyzed using TRIOS or Universal Analysis.

### Polarized Light Microscopy (PLM)

### Leica LM/DM Polarized Light Microscope

Samples were analyzed on a Leica LM/DM polarized light microscope with a digital video camera for image capture. A small amount of each sample was placed on a glass slide, with or without immersion oil, and covered with a glass slip. The sample was viewed with appropriate magnification and partially polarized light, coupled to a λ false-color filter. Images were captured using StudioCapture or Image ProPlus software.

### Hot Stage Microscopy (HSM)

Hot Stage Microscopy was carried out using a Leica LM/DM polarized light microscope combined with a Mettler-Toledo FP82HT hot-stage and a digital video camera for image capture. A small amount of each sample was placed onto a glass slide with individual particles separated as well as possible. The sample was viewed with appropriate magnification and partially polarized light, coupled to a A false-color filter, whilst being heated from ambient temperature, typically at 10° C./min. Data were collected using StudioCapture.

### Gravimetric Vapor Sorption (GVS)

### SMS DVS Intrinsic

Sorption isotherms were obtained using a SMS DVS Intrinsic moisture sorption analyzer, controlled by DVS Intrinsic Control software. The sample temperature was maintained at 25° C. by the instrument controls. The humidity was controlled by mixing streams of dry and wet nitrogen, with a total flow rate of 200 mL/min. The relative humidity was measured by a calibrated Rotronic probe (dynamic range of 1.0-100% RH), located near the sample. The weight change, (mass relaxation) of the sample as a function of % RH was constantly monitored by a microbalance (accuracy±0.005 mg).

Typically, 5-30 mg of sample was placed in a tared mesh stainless steel basket under ambient conditions. The sample was loaded and unloaded at 40% RH and 25° C. (typical room conditions). A moisture sorption isotherm was performed as outlined below (2 scans per complete cycle). The standard isotherm was performed at 25° C. at 10% RH intervals over a 0-90% RH range. Typically, a double cycle (4 scans) was carried out. Data analysis was carried out within Microsoft Excel using the DVS Analysis Suite.

TABLE 1
Method parameters for SMS DVS Intrinsic experiments

| Parameter | Value |
|---|---|
| Adsorption-Scan 1 | 40-90 |
| Desorption, Adsorption- | 90-0, 0-40 |
| Scan 2 | |
| Intervals (% RH) | 10 |
| Number of Scans | 4 |
| Flow rate (mL/min) | 200 |
| Temperature (° C.) | 25 |
| Stability (° C./min) | 0.2 |
| Sorption Time (hours) | 6 hour time out |
| Number of cycles | 2 |

### Hiden IGASorp

Sorption isotherms were obtained using a Hiden IGASorp moisture sorption analyser, controlled by Isochema HISorp software. The sample temperature was maintained at 25° C. by a Grant LT ecocool 150 re-circulating water bath. The humidity was controlled by mixing streams of dry and wet nitrogen, with a total flow rate of 250 ml·min-1. The relative humidity was measured by a calibrated Vaisala RH probe (dynamic range of 0-95% RH), located near the sample. The weight change, (mass relaxation) of the sample as a function of % RH was constantly monitored by the microbalance (accuracy±0.001 mg).

Typically, 20-30 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0-90% RH range. Typically, a double cycle (4 scans) was carried out. Data analysis was carried out within the Isochema HISorp 2019 software and exported into Microsoft Excel to present accordingly.

TABLE 2
Method parameters for Hiden IGASorp experiments

| Parameters | Value |
|---|---|
| Adsorption-Scan 1 | 40-90 |
| Desorption/Adsorption-Scan 2 | 90-0, 0-40 |
| Intervals (% RH) | 10 |
| Number of Scans | 2 |
| Flow rate (ml/min) | 250 |
| Temperature (° C.) | 25 |
| Stability (° C./min) | 0.05 |
| Minimum Sorption Time (mins) | 10 |
| Maximum Sorption Time (mins) | 360 |
| Equilibration Mode | Final Rate |
| Accuracy (tolerance) | +/−0.001 mg/min |
| | (over 600 s) |

### Chemical Purity Determination by HPLC

Purity analysis was performed on an Agilent HP1100/Infinity II 1260 series system equipped with a diode array detector and using OpenLAB software. The full method details are provided below:

TABLE 3
HPLC method for chemical purity determinations

| Parameter | Value | | |
|---|---|---|---|
| Type of method | Reverse phase with gradient elution | | |
| Sample Preparation | 0.5 mg/mL in 100% DMSO | | |
| Column | Supelco Ascentis Express C₁₈ 2.7 μm | | |
| | 100 × 4.6 mm | | |
| Column Temperature (° C.) | 25 | | |
| Injection (μL) | 2 | | |
| Detection: Wavelength, | 255, 90 | | |
| Bandwidth (nm) | | | |
| Flow Rate (mL/min) | 2 | | |
| Phase A | 0.1% TFA in water | | |
| Phase B | 0.085% TFA in acetonitrile | | |
| Timetable | Time (min) | % Phase A | % Phase B |
| | 0 | 95 | 5 |
| | 6 | 5 | 95 |
| | 6.2 | 95 | 5 |
| | 8 | 95 | 5 |

### Water Determination by Karl Fischer Titration (KF)

The water content of each sample was measured on a Metrohm 874 Oven Sample Processor at 150° C. with 851 Titrano Coulometer using Hydranal Coulomat AG oven reagent and nitrogen purge. Weighed solid samples were introduced into a sealed sample vial. Approximately 10 mg of sample was used per titration and duplicate determinations were made. An average of these results is presented unless otherwise stated. Data collection and analysis were performed using Tiamo software.

### Ion Chromatography (IC)

Data were collected on a Metrohm 930 Compact IC Flex with 858 Professional autosampler and 800 Dosino dosage unit monitor, using IC MagicNet software. Accurately weighed samples were prepared as stock solutions in a suitable solvent. Quantification was achieved by comparison with standard solutions of known concentration of the ion being analyzed. Analyses were performed in duplicate and an average of the values is given unless otherwise stated.

TABLE 4
IC method for cation chromatography

| Parameter | Value |
|---|---|
| Type of method | Cation exchange |
| Column | Metrosep C 4-250 (4.0 × 250 mm) |
| Column Temperature (° C.) | Ambient |
| Injection (μl) | Various |
| Detection | Conductivity detector |
| Flow Rate (ml/min) | 0.9 |
| Eluent | 1.7 mM nitric acid |
| | 0.7 mM dipicolinic acid in a |
| | 5% acetone aqueous solution. |

TABLE 5
IC method for anion chromatography

| Parameter | Value |
|---|---|
| Type of method | Anion exchange |
| Column | Metrosep A Supp 5-150 |
| | (4.0 × 150 mm) |
| Column Temperature (° C.) | Ambient |
| Injection (μl) | Various |
| Detection | Conductivity detector |
| Flow Rate (ml/min) | 0.7 |
| Eluent | 3.2 mM sodium carbonate |
| | 1.0 mM sodium hydrogen carbonate |
| | in a 5% acetone aqueous solution |

### Experimental Crystallization Methodologies

The choice of crystallization method has influences which form is produced, and therefore various crystallization methods and conditions may produce different polymorphs.

Crystallization methods used herein are listed in Table 6 together with the degrees of freedom available for each method.

TABLE 6
Crystallization methods

| Method | Degrees of freedom |
|---|---|
| Maturation | Solvent or solvent mixture type, |
| (suspension equilibration or | temperature, ratio of solvent to solid, |
| “slurry ripening”) | solubility, temperature programs, |
| | stirring/shaking rate, incubation time |
| Cooling crystallization | Solvent or solvent mixture type, cooling |
| | profile, temperature at start, temperature |
| | at end, concentration |
| Controlled evaporation | Solvent or solvent mixture type, initial |
| | concentration, evaporation rate, |
| | temperature, pressure, ambient |
| Precipitation by anti-solvent | Solvent, anti-solvent, rate of addition, |
| addition | order of mixing, temperature |
| Desolvation of solvates | “Dry”: temperature, pressure |
| Heat induced crystallizations | Temperature programs (min, max, |
| | gradient) |
| Grinding | Solvent or solvent mixture type, ratio of |
| | solvent to solid, grinding speed. grinding |
| | energy and mode of action |

### Solvent Mediated Techniques

Without wishing to be bound by theory, crystallization occurs when the concentration of a compound in a solvent is higher than its solubility product.

For a crystallization screen, solvents with highly diverse properties were chosen (e.g., hydrogen bond donor/acceptor propensity, dipole moment, dielectric constant, viscosity, etc.). Solvent mixtures were also explored to obtain systems with suitable solubilities, polarities etc. Stability of the compound in the given solvents or solvent mixtures was also considered.

### Maturation/Slurry Ripening

Maturation experiments (or slurry ripening) were performed in various solvents or solvent mixtures and subjected to heat-cool cycles. Without wishing to be bound by theory, repeated heating and cooling cycles may increase the degree of crystallinity or convert a meta-stable state (or out-of-equilibrium state in the case of amorphous material) into a more thermodynamically stable crystalline form. The rate and extent of conversion is dependent upon solubility of the input material.

### Cooling Crystallization

Crystallization was performed by lowering the temperature of a clear solution. Without wishing to be bound by theory, the solubility of most materials decreases with decreasing temperature, so cooling can be used to generate super saturation, thereby resulting in crystallization.

### Controlled Evaporation

Crystallization was performed by controlled evaporation of a clear, particulate free, solution. Without wishing to be bound by theory, controlled evaporation often works well when the solvent has a relatively high vapor pressure, such that the solvent is being removed from the system, thereby increasing the solute concentration.

### Precipitation/Crystallization by Anti-Solvent Addition

Anti-solvent crystallization (or drown out crystallization) is another method that was used to precipitate material from a solution. Without wishing to be bound by theory, the addition of a miscible anti-solvent into a solute solution reduces the original solubility of the solute, increasing saturation and causing its precipitation. Typically, the anti-solvent is selected to be miscible with the solvent at any proportion, and the solute is relatively insoluble in the anti-solvent.

### Example 1

### Summary Salt Form Studies

This study identified salt forms of Structure (I) with suitable solid state and physio-chemical properties for use in a drug product. In parallel, it also investigated solid forms of the free form via a polymorph assessment with a view to identifying forms with appropriate properties for the end use.

Measurements of pKa showed Structure (I) to have a basic pKa at 4.73 and an acidic center at 11.49, and thus the compound was considered a suitable candidate to form salts at either end of the pH scale. Extensive work was carried out to identify salt forms of Structure (I). Studies included solubility and salt formation assessment and four salt screens. A screen with 12 acids was employed and the following techniques were used: heat-cool cycles, cooling ramp, high temperature screen, one and two equivalents targeted screens, and a targeted screen using high purity input material.

During this study, 12 salt forms were identified along with multiple free base forms, two of which were chosen for further development (free form Pattern 3 and Pattern 11). Some of the salts that formed did not display the desired properties. Several salt forms (e.g., phosphate salt Pattern 1, mesylate salt Pattern 1) were characterized as hydrates, however, in each case the associated water molecules were lost at temperatures close to ambient conditions, thus limiting the potential for using these forms in a drug product. It will be appreciated, however, that salt and free base forms with less desirable properties may still be useful in manufacturing processes (e.g., as intermediates) and/or for testing purposes, inter alia, even if they may not be acceptable for use in an approved therapeutic product.

### Example 2

### Salt Screen

Acid and base stock solutions used in the screen were prepared as described in Table 7

TABLE 7
Details of the counter-ion/coformers used in salt screen

| Counter-ion/Co-former | Concentration | Solvent |
|---|---|---|
| Hydrochloric Acid | 1 M | 100% THF |
| Hydrobromic acid | 1 M | 100% THF |
| Sulfuric Acid | 1 M | 100% THF |
| p-Toluene sulfonic acid | 1 M | 100% THF |
| Methane sulfonic acid | 1 M | 100% THF |
| Benzene sulfonic acid | 1 M | 100% THF |
| Maleic acid | 1 M | 100% THF |
| Phosphoric acid | 1 M | 100% THF |
| Tartaric acid | 1 M | 100% THF |
| Fumaric acid | 0.5 M | THF:MeOH 1:1 |
| Citric acid | 1 M | 100% THF |
| Malic acid | 1 M | 100% THF |
| Succinic acid | 1 M | 100% THF |

Structure (I) (20 mg) from Lot A (free form Pattern 1) was charged into HPLC vials, treated with solvent, 10 volumes at a time, until the material fully dissolved or a maximum of 50 volumes had been added. Once 50 volumes was reached without dissolution, the temperature was increased to 50° C. To each suspension, 1.1 molar equiv. (60 μL) of HCl (1M in THF) was added, stirred for 5 min, and slowly cooled to 5° C. at 0.1° C./min and held at that temperature overnight. All solids were then isolated by filtration through PTFE frits and dried under suction for 20 min. The samples were analyzed by XRPD.

Structure (I) Pattern 1 was insoluble in most solvents at each condition. Following the HCl addition, new XRPD patterns were obtained from methanol, ethanol, ethyl acetate, isopropyl acetate, acetonitrile and 90:10 THF:Water. Based on the solubility and the salt formation results, 90:10 THF:Water was selected as the solvent for subsequent salt screens.

TABLE 8
Outcomes of initial solubility testing, x shows where solubilization was not observed,
while ± shows where some solubilization was observed.

| | | | | | | | Upon | Upon addition | Upon | |
|---|---|---|---|---|---|---|---|---|---|---|
| | | 10 | 20 | 30 | 40 | 50 | heating | of 1.1 mol eq. | cooling to | |
| Sample | Solvent | vol | vol | vol | vol | vol | to 50° C. | HCl | 5° C. | XRPD |
| 8-1 | MeOH | x | x | x | x | x | x | Suspension | Suspension | HCl |
| | | | | | | | | | | Pattern 1 |
| 8-2 | EtOH | x | x | x | x | x | x | Suspension | Suspension | HCl |
| | | | | | | | | | | Pattern 2 |
| 8-3 | IPA | x | x | x | x | x | x | Emulsion | Emulsion | — |
| 8-4 | EtOAc | x | x | x | x | x | x | Suspension | Suspension | HCl |
| | | | | | | | | | | Pattern 3 |
| 8-5 | iPrOAc | x | x | x | x | x | x | Suspension | Suspension | HCl |
| | | | | | | | | | | Pattern 3 |
| 8-6 | THF | x | x | x | x | x | x | Emulsion | Emulsion | — |
| 8-7 | MEK | x | x | x | x | x | x | Suspension | Suspension | Poorly |
| | | | | | | | | | | crystalline |
| | | | | | | | | | | (HCl |
| | | | | | | | | | | Pattern 3) |
| 8-8 | Acetone | x | x | x | x | x | x | Emulsion | Emulsion | — |
| 8-9 | MeCN | x | x | x | x | x | x | Suspension | Suspension | HCl |
| | | | | | | | | | | Pattern 3 + |
| | | | | | | | | | | HCl |
| | | | | | | | | | | Pattern 1 |
| 8-10 | 10% | x | x | x | x | x | x | Emulsion | Emulsion | — |
| | water/ | | | | | | | | | |
| | IPA | | | | | | | | | |
| 8-11 | 10% | x | x | ± | ± | ± | ± | Suspension | Suspension | HCl |
| | water/ | | | | | | | | | Pattern 3 + |
| | THF | | | | | | | | | HCl |
| | | | | | | | | | | Pattern 1 |

### Example 3

### Salt Screen 1-THF:Water (9:1)

Poorly Crystalline Structure (I) (from Lot A, Free Form Pattern 1, 30 mg) was dissolved in 50 volumes of THF:Water 9:1 at 60° C. in 13 vials, stirring at 500 RPM (small amount of debris present). The solutions were then treated with 1.1 molar equivalents (90/180 μL) of selected counter-ions (Table 7). The resulting solutions were then cooled down to 5° C. at 0.1° C./min and were held at this temperature for 2 days. The suspensions were filtered and dried under suction for 20 min. The gummed sample was left with the cap off to promote crystallization. All solids obtained were initially analyzed by XRPD and stored in glass HPLC vials for subsequent analysis. All solids that displayed new XRPD diffractograms were characterized further using some or all of DSC, HPLC, ¹H NMR, and storage at elevated temperature and humidity.

Solid material was isolated from all the samples suggesting that the compound of Structure (I) readily forms salts. Based on the XRPD analysis of the solids, names have been assigned to the forms according to Table 9. Further characterization and analysis of these solids can be found in the disclosures and FIGs herein.

TABLE 9
Observations made during the THF:Water 9:1 salt screen

| Sample | | Upon addition | Upon cooling | |
|---|---|---|---|---|
| ID: | Acid | of acid at 60° C. | to 5° C. | XRPD |
| 9-1 | Hydrobromic acid | Suspension | Suspension | HBr Pattern 1 |
| 9-2 | Hydrochloric acid | Suspension | Suspension | HCl Pattern 1 + |
| | | | | HCl Pattern 3 |
| 9-3 | Sulfuric acid | Suspension | Suspension | Sulfate |
| | | | | Pattern 1 |
| 9-4 | p-Toluenesulfonic | Suspension | Suspension | Tosylate |
| | acid | | | Pattern 1 |
| 9-5 | Methane sulfonic | Almost solution | Suspension | Mesylate |
| | acid | | | Pattern 1 |
| 9-6 | Benzene sulfonic | Suspension | Suspension | Besylate |
| | acid | | | Pattern 1 |
| 9-7 | Maleic acid | Almost solution | Suspension | Maleate |
| | | | | Pattern 1 |
| 9-8 | Phosphoric acid | Suspension | Suspension | Phosphate |
| | | | | Pattern 1 |
| 9-9 | L-Tartaric acid | Almost solution | Suspension | Tartrate |
| | | | | pattern 1 |
| 9-10 | Fumaric acid | Almost solution | Suspension | Fumarate |
| | | | | pattern 1 |
| 9-11 | Citric acid | Almost solution | Suspension | Citrate |
| | | | | pattern 1 |
| 9-12 | L-Malic acid | Almost solution | Solution with | Free Form |
| | | | gum | Pattern 2 |
| 9-13 | Succinic acid | Almost solution | Suspension | Free Form |
| | | | | Pattern 2 |

### Example 4

### Salt Screen 2—High Temperature Solvent Screen

Due to low solubility observed in the experiments of Example 2, a second solubility assessment of Structure (I) from Lot A was carried out using higher temperatures closer to the boiling points of the selected solvent systems (Table 10). Once at temperature, phosphoric acid or a base was added to either promote formation of a phosphate or Na/K salt.

Structure (I) (from Lot A, Free Form Pattern 1, 30 mg) was weighed into 6 HPLC vials and a stirrer bar added to each one. 50 volumes of different solvents were added to the samples according to Table 10 and the samples heated incrementally on a Polar Bear heating block, stirring at 400 RPM. Observations were noted as the temperature was increased, holding at each temperature for 10 minutes.

All samples were subsequently cooled to 60° C. and Samples 10-1 through 10-5 had 1.1 molar equivalents, 90 μL of phosphoric acid (1M in THF) added. These were allowed to equilibrate, cooled to 5° C. at 0.1° C./min, and held stirring for 14 hours (400 rpm). Sample 10-6 had no acid added but was treated to the same cooling profile. Vials with Samples 10-7 and 10-8 had 0.8 molar equivalents of NaOH and KOH solutions (1M in water) added before being allowed to equilibrate and being treated to the same cooling profile.

Suspensions were isolated using a positive pressure and filter cartridges with PE frits. The resulting solids were dried under compressed air and analyzed by XRPD.

All solids that displayed new XRPD diffractograms were characterized further using DSC, HPLC and ¹H NMR and are discussed herein.

The observations made during the solubility assessment are presented in Table 10. Despite the increased temperatures, none of the solvent systems exhibited full dissolution (there was still some debris present in the THF:water 9:1 sample).

TABLE 10
Observations during the high temperature solubility assessment

| Sample ID | Solvent | 60° C. | 70° C. | 80° C. | 85° C. |
|---|---|---|---|---|---|
| 10-1 | Acetone:Water (9:1) | X | X | — | — |
| 10-2 | 1,4-Dioxane | X | X | X | X |
| 10-3 | EtOH | X | X | X | X |
| 10-4 | EtOAc | X | X | X | X |
| 10-5 | EtOH:Water (9:1) | X | X | X | X |
| 10-6 | THF:Water (9:1) | T | T | — | — |
| 10-7 | THF:Water (9:1) | T | T | — | — |
| 10-8 | THF:Water (9:1) | T | T | — | — |

T = turbid solution
X = suspension

Addition of 1.1 mol eq. of phosphoric acid to Samples 10-1 through 10-5 resulted in the isolation of phosphate (PHO) Pattern 1 from ethyl acetate, PHO Pattern 1 with some additional peaks from acetone:water 9:1 and ethanol:water 9:1 and a new form PHO Pattern 2 from 1,4-dioxane. Sample 10-6, with no added counter ion, yielded Free Form Pattern 2. The samples to which base was added gave brown oily/gummy residues on the vial walls. These results are summarized in Table 11 and further characterization for PHO Pattern 2 can be found in the disclosures and FIGs herein.

TABLE 11
Outcomes of high temperature solution testing

| | | Counter ion | XRPD/ |
|---|---|---|---|
| Sample ID | Solvent | addition | Observation |
| 10-1 | Acetone:Water | 90 μL of H₃PO₄ | Phosphate |
| FIG. 20 | (9:1) | (1 M in THF) | pattern 1, with |
| top trace | | | additional peaks |
| 10-2 | 1,4-dioxane | 90 μL of H₃PO₄ | Phosphate |
| FIG. 20 2^{nd} | | (1 M in THF) | pattern 2 |
| from top trace | | | |
| 10-3 | EtOH | 90 μL of H₃PO₄ | Emulsion |
| | | (1 M in THF) | |
| 10-4 | EtOAc | 90 μL of H₃PO₄ | Phosphate |
| FIG. 20 3^{rd} | | (1 M in THF) | pattern 1 |
| fromt op trace | | | |
| 10-5 | Ethanol:Water | 90 μL of H₃PO₄ | Phosphate |
| FIG. 20 4^{th} | (9:1) | (1 M in THF) | pattern 1 with |
| from top trace | | | additional peaks |
| 10-6 | THF:Water | Control | Free Form |
| | (9:1) | | Pattern 2 |
| 10-7 | THF:Water | 66 μL of NaOH | Oil/gum coating |
| | (9:1) | (1 M in H₂O) | on vial |
| 10-8 | THF:Water | 66 μL of KOH | Oil/gum coating |
| | (9:1) | (1 M in H₂O) | on vial |

Characterization of Sample 10-6 is summarized in Table 12. The XRPD showed isolation of a new crystalline form, Free Form Pattern 2. The crystallization gave material with a purity of 98.2%, but the material still contained 0.2 mole equivalents of potassium, which were also present in the input material.

TABLE 12
Characterization summary for Free Form Pattern 2, Sample 10-6

| Sample ID | Sample 10-6 |
|---|---|
| XRPD Form | Free Form Pattern 2 |
| HPLC | 98.2% |
| (Generic method, DMSO) | |
| IC (Potassium) | 0.2 mol eq. |

### Example 5

### Salt Screen 3-Double Equivalent Salt Solution

To encourage the formation of different salt forms, another screen was carried out, predominantly in THF:water 9:1. This screen used an additional equivalent of each of the selected acids.

Poorly crystalline Structure (I) (from Lot A, free form Pattern 1, 20 mg) was dissolved in 50 volumes of the solvent system at 60° C. in 13 vials (Samples 13-1 through 13-13) and in DMSO at 60° C. in vial 14 (Sample 13-14). The solutions were then treated with 2.1 molar equivalents of the selected counter-ions according to Table 13. The resulting solutions were then cooled down to 5° C. at 0.1° C./min and were held at this temperature for 2 days. Stirring (500 rpm) was maintained throughout. The suspensions were isolated using PE frits and filter cartridges.

The addition of 2.1 mol eq. of acid counter ion led to the isolation of a large number of solids, three of which had not been observed before. These were HCl Pattern 4, Mesylate Pattern 2 and Malate Pattern 2, and the further characterization of these forms can be found in the disclosures and FIGs herein. Also noted was the poor filterability of some of the suspensions, which is likely a function of particle size.

TABLE 13
Outcomes and observations of double equivalent salt addition testing

| Sample ID | Counter ion addition | Final observations | XRPD |
|---|---|---|---|
| 13-1 | Hydrobromic acid | Fine suspension poorly filterable | HBr pattern 1 |
| 13-2 | Hydrochloric acid | Fine suspension poorly filterable | HCI pattern 4 |
| 13-3 | Sulfuric acid | Fine suspension poorly filterable | Sulfate pattern 1 |
| 13-4 | p-Toluenesulfonic acid | Fine suspension poorly filterable | Tosylate pattern 1 |
| 13-5 | Methane sulfonic acid | Suspension, filterable | Mesylate pattern 2 |
| 13-6 | Benzene sulfonic acid | Suspension, filterable | Besylate pattern 1 |
| 13-7 | Maleic acid | Fine suspension poorly filterable | Maleate pattern 1 |
| 13-8 | Phosphoric acid | Fine suspension poorly filterable | Phosphate pattern |
| | | | 1 |
| 13-9 | L-Tartaric acid | Fine suspension poorly filterable | Few broad, weak |
| | | | peaks |
| 13-10 | Fumaric acid | Fine suspension poorly filterable | Fumarate pattern |
| | | | 1, low crystallinity |
| 13-11 | Citric acid | Fine suspension poorly filterable | Citrate pattern 1 |
| 13-12 | L-Malic acid | Fine suspension poorly filterable | Malate pattern 2 |
| 13-13 | Succinic acid | Light turbid suspension | Free form pattern |
| | | | 2, with additional |
| | | | peaks |
| 13-14 | Hydrochloric acid | Light turbid suspension, | N/A |
| | | isolation not possible | |

### Example 6

### Characterization of Solids from Screens 1-3

The materials generated in screens 1-3 were characterized to determine the nature of the solids.

TABLE 14
Characterization summary for HBr Pattern 1 and HCI Pattern 1 and 3

| Sample ID | 9-1 | 9-2 |
|---|---|---|
| XRPD Form | HBr Pattern 1 | HCI Patterns 1 and 3 |
| ¹H NMR | 0.13 mol eq. THF. Anion not | 0.12 mol eq. THF. Anion not |
| (in d6-DMSO) | visible in NMR, salt formation | visible in NMR, salt formation |
| | implied by changes in aromatic | implied by changes in aromatic |
| | region. | region. |
| HPLC (Generic | 98.1% purity | 98.3% purity |
| method, DMSO) | | |
| DSC | Two broad endotherms, first onset | Endotherm with onset 76.5° C. |
| | at 43.4° C., 32 J/g and second onset | (13 J/g) leading into sharper |
| | at 99.1° C., 29 J/g. One exotherm | large endotherm onset |
| | onset at 246.9° C., 2 J/g. | 124.3° C. (45 J/g) |
| XRPD and HPLC | XRPD-HBr Pattern 1 | No change by XRPD |
| post storage at | 98.7% purity | 97.7% purity |
| 40° C./75% RH | | |
| (7 days) | | |
| IC | 0.9 equivalents of bromide | 0.81 equivalents of chloride |
| | counter-ion observed. Note: | counter-ion observed. |
| | Interaction of bromine with | |
| | DMSO solvent has led to a change | |
| | in peak-shape | |
| Tentative | Salt formed with complex thermal | Polymorphic as seen in the |
| Assignment | behavior but not stable to storage | solubility screen and appears to |
| | at 40° C./75% RH | be a mixture of forms. |
| | | Reasonable thermal behavior |
| | | but with a low melt. |

HBr Pattern 1 contained some diffraction peaks matching the input material, and the thermal behavior was likely not suitable for use as a drug product, containing two broad endotherms and no clear melt. The sample remained HBr Pattern 1 after storage and a slight uplift in purity was observed.

HCl Patterns 1 and 3 appeared to be a mixture of forms based on the XRPD patterns observed in the salt formation assessment. The thermal analysis contained two endotherms, a smaller broad endotherm followed by a sharper endotherm which could be a melt. Only 0.8 mol equivalents of chloride were observed in the ion chromatography. The sample was stable to storage at 40° C./75% RH.

TABLE 15
Characterization summary for sulfate (SUL) Pattern 1 and tosylate (TOS) Pattern 1

| Sample ID | 9-3 | 9-4 |
|---|---|---|
| XRPD Form | SULFATE Pattern 1 | TOSYLATE Pattern 1 |
| ¹H NMR | 0.04 mol eq. THF. Anion not visible | 0.42 mol eq. THF. 1.03 mol eq. |
| (in d6-DMSO) | in NMR, salt formation implied by | of tosylate. |
| | changes in aromatic region. | |
| HPLC | 97.4% purity | 98.6% purity |
| (Generic method, | | |
| DMSO) | | |
| DSC | Two broad endotherms. First onset | One broad endotherm, onset |
| | at 116.3° C., 38 J/g, second | ~42° C., 165 J/g. Two exotherms, |
| | onset at 206.0° C., 12 J/g. | broad first onset at 195.1° C., 14 |
| | | J/g, sharp second onset at |
| | | 236.9° C., 23 J/g. |
| XRPD and HPLC | 95.6% purity. SUL Pattern 1 | 99.3% purity. Loss of |
| post storage at | maintained. | crystallinity, possibly some Free |
| 40° C./75% RH | | Form Pattern 1 |
| IC | 0.96 equivalents of sulfate counter- | N/A |
| | ion observed. | |
| Tentative | Salt formed with reasonably | Salt formed with complex |
| Assignment | complex thermal data, loss of purity | thermal data and not stable when |
| | on storage. | stored at 40° C./75% RH |

SUL Pattern 1 contained 1 mole equivalent of sulfate by ion chromatography and a purity of 97.4% by HPLC. The DSC contained two broad endotherms, the shape of the first one suggesting that a second event may be occurring simultaneously. No clearly defined melting behavior was observed. The solid form was stable to storage at 40° C./75% RH, but there was a decrease in the observed chemical purity.

TOS Pattern 1 contained 1 mole equivalent of tosylate by ¹H NMR which also showed 0.42 equivalents of THE present, not precluding the possibility the solid form is solvated. The DSC contained a large endotherm from 40-140° C. (165 J/g) which likely represented this solvent loss. The DSC also contained two exotherms at higher temperature with no obvious melt. After storage at 40° C./75% RH, there was a large loss in crystallinity with a possibility of some Free Form Pattern 1 being present.

TABLE 16
Characterization summary for Free Form Pattern 3 and besylate (BES) Pattern 1

| Sample ID | 9-5 | 9-6 |
|---|---|---|
| XRPD Form | Free Form Pattern 3 (formerly | BESYLATE Pattern 1 |
| | identified as MESYLATE | |
| | Pattern 1) | |
| ¹H NMR | 0.15 Eq THF. 0.19 mol eq. of | 0.18 Eq. THF. 1.03 mol eq. of |
| (in d6-DMSO) | mesylate. | besylate. |
| HPLC | 99.1% purity | 98.1% purity. |
| (Generic method, | | |
| DMSO) | | |
| DSC | Two broad endotherms, first onset | One broad, split endotherm. |
| | at 89.9° C., 31.2 J/g, second onset at | Onset at 52.2° C., 32 J/g, splitting |
| | 160.9° C., 2 J/g. One broad | at 68.4° C., 70 J/g. One broad, |
| | exotherm, onset at 202.7° C., 24 J/g | split exotherm. Onset at 199.9° C., |
| | | 8 J/g, splitting at 206.7° C., 10 J/g. |
| XRPD and HPLC | 98.5% purity. Free Form Pattern 3 | 98.8% purity. No evidence of |
| post storage at | retained. | crystallinity. |
| 40° C./75% RH | | |
| IC | N/A | N/A |
| Tentative | Free Form | Salt formed with complex thermal |
| Assignment | | data. Form not stable to storage at |
| | | 40° C./75% RH |

Sample 9-5 was originally assigned as mesylate (MES) Pattern 1 but after characterization and comparison with other data collected, it was determined to be Free Form Pattern 3. Only 0.19 mole equivalents of mesylate were observed by NMR along with 0.15 equivalents of THF. The material remained unchanged by XRPD after storage at 40° C./75% RH with a small decrease in chemical purity.

Sample 9-6, denoted BES Pattern 1, was found to contain 1 mole equivalent of benzenesulfonate by ¹H NMR spectroscopy. The sample contains 0.18 mole equivalents of THF. The residual solvent likely contributed to the overlapping endotherms seen in the DSC between 50-120° C. The DSC also contained 2 exotherms at high temperatures. The solid form was not stable to storage at 40° C./75% RH.

TABLE 17
Characterization summary for maleate (MALe) Pattern 1, phosphate (PHO) Pattern 1
and tartrate (TAR) Pattern 1

| Sample ID | 9-7 | 9-8 | 9-9 |
|---|---|---|---|
| XRPD Form | MALEATE Pattern 1 | PHOSPHATE Pattern 1 | TARTRATE Pattern 1 |
| ¹H NMR | 0.25 eq. THF. 1.19 mol | 0.11 eq. THF. Anion not | 0.11 eq. THF. 1 mol |
| (in d6-DMSO) | eq. of maleate. | visible in NMR. | eq. of tartrate. |
| HPLC | 97.8% purity. | 98.0% purity. | 98.1% purity. |
| (Generic | | | |
| method, | | | |
| DMSO) | | | |
| DSC | Two endotherms. First, | Two broad endotherms. | Two broad |
| | broad, onset at | First onset at 76.2° C., | endotherms. First |
| | 126.8° C., 3 J/g. Second, | 213 J/g. Second onset at | onset at ~37° C., 64 J/g. |
| | sharp, onset at 226.2°, | 207.6° C., 40 J/g. | Second onset at |
| | 113 J/g. | | 207.4° C. 7 J/g, with a |
| | | | split at 207.6° C., 15 |
| | | | J/g. |
| XRPD and | 98.8% purity. Maleate | 98.5% purity. Phosphate | 98.1% purity. Tartrate |
| HPLC post | pattern retained; very | pattern retained, | pattern retained. |
| storage at | low intensity due to | possible reduction in | |
| 40° C./75% RH | low sample loading | crystallinity. | |
| IC | N/A | 1.27 equivalents of | N/A |
| | | phosphate counter-ion | |
| | | observed. | |
| Tentative | Salt formed with | Straightforward thermal | Similarities in XRPD |
| Assignment | straightforward thermal | data. Form looks stable | to CIT Pattern 1 may |
| | data. Form looks stable | at higher temp/humidity | point towards this |
| | at higher | with a possible | being a free form. |
| | temp/humidity with a | reduction in | |
| | possible reduction in | crystallinity. | |
| | crystallinity. | | |

Maleate Pattern 1 contained 1.2 mol eq. of maleate by ¹H NMR spectroscopy along with 0.25 mol eq. of residual THF. The salt form had reasonably straightforward thermal data with a small exotherm at 126.8° C. and larger overlapping events with onset at 226.2° C. The XRPD after storage at 40° C./75% RH for 7 days had very low intensity, possibly due to low sample loading, and the observed reflections were consistent with Maleate Pattern 1.

Phosphate Pattern 1 was obtained with a HPLC purity of 98.0% and the solid contained 0.11 mol eq. of THF. IC showed that the solid contained 1.27 mol eq. of phosphate. The DSC contained a large broad endotherm between 50-150° C. (212.5 J/g), which was followed by a smaller broad endotherm with an onset at 207.6° C. (39.5 J/g). The XRPD after storage at 40° C./75% RH for 7 days had low intensity, possibly due to low sample loading, and the observed reflections were consistent with PHO Pattern 1.

Tartrate Pattern 1 had an XRPD diffractogram consistent with that of citrate (CIT) Pattern 1 (Table 18). 1 mole equivalent of tartaric acid was observed in the ¹H NMR spectrum with trace amounts of THF. The DSC contained a broad endotherm from 40-150° C. with two overlapping exotherms at just above 200° C. The solid form is stable to storage at elevated temperature and humidity conditions. Since CIT Pattern 1 (in Table 18 below) did not appear to be a salt yet shared a XRPD pattern with TAR Pattern 1, TAR Pattern 1 may be a free form.

TABLE 18
Characterization summary for fumarate (FUM) Pattern 1 and citrate (CIT) Pattern 1

| Sample ID | 9-10 | 9-11 |
|---|---|---|
| XRPD Form | FUMARATE Pattern 1 | CITRATE Pattern 1 |
| ¹H NMR | 0.53 eq THF. 1.83 mol eq. | 0.15 Eq THF. No evidence of citrate |
| (in d6-DMSO) | fumarate. | salt formation in NMR. |
| HPLC | 98.1% purity. | 98.2% purity. |
| (Generic | | |
| method, | | |
| DMSO) | | |
| DSC | One broad endotherm, onset at | Three broad endotherms. First onset at |
| | 180.5, 54 J/g. | 42.5° C., 49 J/g. Second onset at |
| | | 129.0° C., 2 J/g. Third onset at |
| | | 162.7° C., 180 J/g. |
| XRPD and | 98.8% purity. Fumarate Pattern 1 | 98.8% purity. Citrate Pattern 1 |
| HPLC post | retained. | retained. |
| storage at | | |
| 40° C./75% RH | | |
| IC | N/A | N/A |
| Tentative | Salt formed although stoichiometry | Free Form |
| Assignment | is not clear. Good thermal profile | |
| | and stable to static storage | |

FUM Pattern 1 was obtained with a HPLC purity of 98.1%. The ¹H NMR spectrum showed the material contained 1.83 mole equivalents of fumarate, which was not consistent with formation of a salt with standard stoichiometry. The material also contained 0.5 mol equivalents of THE, which may indicate that it was a solvated form. The DSC contained a single endotherm with an onset at 180.5° C., although the shape of this event suggested that this could be 2 overlapping events. The solid form was stable to storage at 40° C./75% RH for 7 days, with a slight uplift observed in the HPLC of the sample. One possibility is that a co-crystal was formed, which may or may not contain any charged species.

Sample 9-11, denoted CIT Pattern 1, had a XRPD pattern matching that for TAR Pattern 1, described above, which was assigned to be a mono-salt. Sample 9-11 did not contain any citrate by ¹H NMR spectroscopy, suggesting it could be a free form.

TABLE 19
Characterization summary of Free Form Pattern 2

| ID | 9-12 | 9-13 |
|---|---|---|
| XRPD Form | Free Form Pattern 2 (formerly | Free Form Pattern 2 (formerly |
| | MALATE, MALi Pattern 1) | SUCCINATE, SUC Pattern 1) |
| ¹H NMR | 4.1 eq. THF. No evidence of malate | 0.45 eq. THF. 0.48 mol eq. |
| (in d6-DMSO) | salt formation in NMR. | succinate. |
| HPLC | 97.2% purity | Insufficient material for |
| (Generic | | HPLC analysis. |
| method, DMSO) | | |
| DSC | Two broad endotherms. First onset | One broad endotherm, onset |
| | at 48.4° C., 22 J/g. Second onset | 157.0° C., 11 J/g. One sharp |
| | 120.5° C., 44 J/g, splits at ~155° C., | exotherm, onset |
| | 15 J/g. One broad exotherm, onset at | 251.0° C., 1 J/g. |
| | 207.8° C., 38 J/g. | |
| XRPD and | 98.8% purity. No observed | Insufficient material. |
| HPLC post | crystallinity. Note: This observed | |
| storage at | lack of crystallinity could be due to | |
| 40° C./75% RH | lack of available material. | |
| IC | N/A | N/A |
| Tentative | Free Form | Free Form |
| Assignment | | |

Sample 9-12 was originally denoted MALi Pattern 1 but comparison with subsequent data and the lack of malate observed in the ¹H NMR led to the re-assignment as Free Form Pattern 2. The XRPD pattern for Sample 9-13 matches well with Free Form Pattern 2 so was also reassigned on this basis. Both samples had residual THE present 4.1 and 0.5 mole equivalents respectively, suggesting this could be a solvated form.

TABLE 20
Characterization summary for phosphate (PHO) Pattern 2 and HCI Pattern 4

| Sample ID | 10-2 | 13-2 |
|---|---|---|
| XRPD Form | PHOSPHATE | HCl Pattern 4 (Polyethylene peaks |
| | (PHO) Pattern 2 | from frit present) |
| ¹H NMR | Anion not visible in NMR, no | 2.263 mol. eq. of THF. Salt formation |
| (in d6- | residual solvent | implied by change in aromatic region. |
| DMSO) | | |
| HPLC | 96.2% Purity. | 95.0% purity. |
| (Generic | | |
| method, | | |
| DMSO) | | |
| DSC | Three broad endotherms. First onset | One sharp endotherm, onset at |
| | at ~30° C., 40.3 J/g. Second onset at | 123.7° C., 111 J/g. |
| | 145.8° C., 2.8 J/g, split at 163.8° C., | |
| | second half 12.0 J/g. Third onset at | |
| | 191.6° C, 30.5 J/g. | |
| XRPD and | XRPD shows notable reduction in | XRPD peaks not attributed to filter |
| HPLC post | crystallinity. 97.0% purity observed. | frit are not present. 95.7% purity |
| storage at | | |
| 40° C./75% | | |
| RH | | |
| IC | 1.1 mol. eq of phosphate counter-ion | Insufficient material |
| | observed. | |
| Tentative | Phosphate salt formation is evident | Despite straightforward thermal |
| Assignment | but shows complex thermal | behavior, low purity and low |
| | behavior and low purity. | solubility make this pattern less |
| | | viable |

PHO Pattern 2 was obtained from 1,4-dioxane in Salt Screen 2. It exhibited complex thermal behavior with a broad endotherm between 30-120° C., followed by a series of other endothermic events. The material has 1.1 equivalents of phosphate by IC and lost a significant amount of crystallinity on storage at 40° C. 75% RH for 7 days.

HCl Pattern 4 was poorly crystalline, with the largest two peaks being attributed to contamination from the filter frit (21.5° and 24.0° 20). The DSC contained one sharp endotherm with onset at 123.7° C., which was attributed to the polyethylene filter frit. The sample also contained a large amount of residual THF by ¹H NMR spectroscopy. The poor crystallinity and instability to storage at elevated temperature and humidity indicated that this salt form was likely not suitable for use in a drug product.

TABLE 21
Characterization summary for mesylate (MES) Pattern 2 and malate (MALi) Pattern 2

| Sample ID | 21-1 | 21-2 |
|---|---|---|
| XRPD Form | MES Pattern 2 | MALi Pattern 2 |
| ¹H NMR | 4 mol. eq. of THF. 2.9 mol. | 0.82 mol. eq. of THF. No evidence |
| (in d6-DMSO) | Eq. of mesylate. | of malate salt formation in NMR. |
| HPLC | 93.2% purity. | 98.0% purity. |
| (Generic | | |
| method, DMSO) | | |
| DSC | One broad endotherm, onset at | Two broad endotherms. First onset |
| | 62.5° C., 150.5 J/g. One broad | at 56.6° C., 57.5 J/g. Second onset |
| | exotherm, onset at 235.6° C., 34 | at 184.7° C., 82.1 J/g, splits at |
| | J/g. | ~208° C., ~219° C. and ~245° C. |
| Tentative | Notable increase in purity after | NMR shows no malate. Suggests |
| Assignment | storage, with no observed impact | free form. |
| | on crystallinity. Relatively | |
| | straightforward thermal behavior. | |

MES Pattern 2 was obtained from the double equivalents screen and had a relatively low purity of 93.2%. It had a high residual solvent content, reflected in the large, broad endotherm at the start of the DSC. At high temperature, >200° C. there was a complex endo-exotherm.

MALi Pattern 2 was obtained from the double equivalents screen and was likely a free form, as there are no peaks attributed to L-malate in the ¹H NMR spectrum. It had a large amount of residual THF (0.82 equivalents) which was also seen in DSC as a large, broad endotherm with an onset at 65.6° C. There were a large number of overlapping events from 180-260° C.

### Example 7

### Summary of Salt Screens 1-3

A solubility and preliminary salt formation assessment, along with three salt screens were carried out using the compound of Structure (I) Pattern 1 (from Lot A) as input material. Two of the screens were carried out in THF:Water 9:1, as this was the only solvent system identified with any appreciable solubility. The third screen tried to circumvent this by increasing the temperature used for selected solvents.

Through these screens a total of 11 salt forms have been crystallized and characterized. Additionally, 2 new free form patterns were also identified, Free Form Pattern 2 and Free Form Pattern 3. Three further samples were isolated where the nature of the form was not definitive (TAR Pattern 1, CIT Pattern 1 and MALi Pattern 2).

In general, the solid form properties of the salts isolated from screens using Structure (I) from Lot A were not desirable for development as a drug product (e.g., due to complex or unfavorable thermal behavior or instability under certain storage conditions) but may nevertheless have utility as intermediates in a manufacturing process and/or for testing purposes. It was postulated that the 0.2 equivalents of potassium in the input material from Lot A may have impacted the properties of the resulting materials.

### Example 8

### Screen 4—Targeted Salt Screen

An additional targeted salt screen was conducted on the compound of Structure (I), using material from Lot B, which had lower residual potassium content and was characterized as being Free Form Pattern 3 (see Example 20 below). Attempts were made to form salts using the most promising candidates from previous screening: hydrochloric acid, methane sulfonic acid, phosphoric acid, fumaric acid and sulfuric acid using THF:water (9:1) or 1,4-Dioxane.

The compound of Structure (I) (from Lot B, ca. 30 mg, Free Form Pattern 3) was treated with increasing aliquots of THF:water (9:1) or 1,4-dioxane up to 70 vol (2.1 mL) or until dissolution was achieved at 50° C., 500 rpm. After each solvent addition, observations were recorded. Samples that did not dissolve were heated to 70° C., 500 rpm with further observations taken at 70° C.

Each sample was then subjected to 1.1, 2.1, or 3.1 mol equiv. of selected free acids (Table 22), depending on the target stoichiometry. Observations were taken.

Samples were then cooled from 70 to 5° C. at 0.1° C./min and held at 5° C. overnight, 500 rpm. Further observations were taken, and the solid material was isolated using an SPE syringe cartridge and frit under positive pressure after 24 hours.

Any solids obtained were analyzed by XRPD, ¹H NMR, HPLC, TGA, DSC, XRPD post storage at 40° C./75% RH after 7 days & IC if necessary. Additional microscopy techniques (PLM & HSM) were used to complement the above analysis where appropriate.

TABLE 22
List of counterions and target salt forms

| Sample | Counter ion | Mole Equiv. of | Target |
|---|---|---|---|
| ID | (concentration) | acid added | Patterns |
| 22-1 | HCl (1M) | 1 | HCl P1 + P3 |
| 22-2 | Phosphoric Acid (1M) | 1 | PHO P1 |
| 22-3 | Phosphoric Acid (1M) | 1 | PHO P2 |
| 22-4 | Fumaric Acid (0.5M) | 1 | FUM P1 |
| 22-5 | Fumaric Acid (0.5M) | 2 | FUM P1 |
| 22-6 | Methanesulfonic Acid (1M) | 2 | MES P2 |
| 22-7 | Methanesulfonic Acid (1M) | 3 | MES P2 |
| 22-8 | Sulfuric acid (1M, THF) | 1.1 | SUL P1 |

P = pattern

### Results and Discussion

The results from the targeted salt screen are outlined in Table 23.

TABLE 23
Observations and outcomes of targeted salt addition screen

| | | | | | | | Obs. after | Obs. | |
|---|---|---|---|---|---|---|---|---|---|
| | | | | | | Obs. | counter | after | |
| Sample | Target | | | Mole | | At | ion | cooling | |
| ID | Form | Solvent | Acid | Eq. | Vol | 70° C. | addition | to 5° C. | XRPD |
| 22-1 | HCl | THF/Water | HCl (1M) | 1 | 70 | S | S | S | HCl |
| | P1 + | (10%) | | | | | | | P1 |
| | P3 | | | | | | | | |
| 22-2 | PHO | THF/Water | Phosphoric Acid | 1 | 70 | S | S | S | PHO |
| | P1 | (10%) | (1M) | | | | | | P1 |
| 22-3 | PHO | 1,4- | Phosphoric Acid | 1 | 70 | S | S | S | PHO |
| | P2 | Dioxane | (1M) | | | | | | P1 |
| | | | | | | | | | (WD) |
| 22-4 | FUM | THF/Water | Fumaric Acid | 1 | 70 | S | S | Yellow | FUM |
| | P1 | (10%) | (0.5M) | | | | | S | P2 |
| 22-5 | FUM | THF/Water | Fumaric Acid | 2 | 70 | S | S | Yellow | FUM |
| | P1 | (10%) | (0.5M) | | | | | S | P2 |
| 22-6 | MES | THF/Water | Methanesulfonic | 2 | 70 | S | S | Yellow | MES |
| | P2 | (10%) | Acid (1M) | | | | | S | P2 |
| 22-7 | MES | THF/Water | Methanesulfonic | 3 | 70 | S | S | Yellow | MES |
| | P2 | (10%) | Acid (1M) | | | | | S | P2 |
| 22-8 | SUL | THF/Water | Sulfuric acid | 1.1 | 70 | S | Palc | Pale | SUL |
| | P1 | (10%) | (1M, THF) | | | | Yellow S | Yellow | P1 + |
| | | | | | | | | S | extra |
| | | | | | | | | | peaks |

Key: S = Suspension, Obs. = Observations, P = Pattern, WD = weak diffraction

As can be seen from Table 23, no sample dissolved at 70° C. at 70 vol.

This is in contrast to the batch of material used in the previous screen (i.e., with material from Lot A characterized as poorly crystalline Free Form Pattern 1) where dissolution was achieved. The higher crystallinity and different solid form of the input material used in these experiments may have impacted the results. As a consequence of insolubility, complete dissolution of API could not be achieved prior to counter ion addition. Therefore, the salt formations form this screen were the product of slurry conversions.

From previous screening, Chloride Pattern 1 and Pattern 3, Phosphate Pattern 1 and Pattern 2, Fumarate Pattern 1 and Mesylate Pattern 2 were targeted. These salts were deemed to have promising solid form properties. Conditions used for salt formation were therefore replicated for these systems. Some color change to the suspensions from white to yellow was observed on addition of fumaric acid, methanesulfonic acid, and sulfuric acid.

XRPD analysis of the solid materials were conducted after 24 hours. From the targeted salt screening, the following patterns were obtained: HCl Pattern 1, PHO Pattern 1, MES Pattern 2 and a novel FUM form (Pattern 2)

Samples were characterized by XRPD, ¹H NMR, HPLC, TGA, DSC, XRPD post storage at 40° C./75% RH after 7 days & IC. Additional microscopy techniques (PLM & HSM) were used to complement the above analysis where appropriate.

The results for Chloride Pattern 1 and Phosphate Pattern 1 are summarized in Table 24.

TABLE 24
Characterization of Chloride Pattern 1 and Phosphate Pattern 1

| Sample ID | 22-1 | 22-2 |
|---|---|---|
| XRPD | HC1 P1 | PHO P1 |
| ¹H NMR | N/A | Consistent with structure. Residual |
| (in d6-DMSO) | | THF detected (<0.1 mol equiv.) |
| HPLC | 99.5% | 98.8% |
| (Generic method, | | |
| DMSO) | | |
| TGA | A weight loss of 8 wt % from 35 | A weight loss of 7 wt % from 35 to |
| | to 150° C. (equiv. to 1.9 mol of | 150° C. (equiv. to 1.9 mol of |
| | water or 0.5 mol of THF). | water). Decomposition above ca. |
| | Decomposition above ca. 250° C. | 250° C. |
| DSC | Large broad endotherm with an | Large broad endotherm with an |
| | onset of 67.2° C. (216 J/g). | onset of 82.1° C. (231 J/g). 2nd |
| | Exotherm with a onset of 211.5° C. | endotherm with an onset of |
| | (8 J/g)* | 207.8° C. (42 J/g) |
| XRPD post | Unchanged, 99.3% | Unchanged, 99.1% |
| storage at 40° C./ | | |
| 75% RH, 7 Days | | |
| IC | 0.5 mol equiv. of chloride (4.6 | 1.0 mol equiv. of phosphate (20.9 |
| | wt %, adjusted for water content) | wt %, adjusted for water content) |
| PLM | N/A | Thin needles, generally <75 μm in |
| | | length |
| HSM (up to | N/A | Unchanged up to 260° C., where |
| 270° C.) | | the material begins to melt |
| Tentative | Possible mix of free form and | Mono-phosphate hydrate |
| Assignment | chloride hydrate | |

*Note
that DSC of Sample 22-1 is not consistent with previous sample (Sample 9-2, a mix of P1 and P3)

From the table, Sample 22-1 (Chloride Pattern 1) displayed an XRPD pattern consistent with previous samples, however the thermal behavior was not consistent with a previous sample (Sample 9-2, P1+P3), showing a larger broad endotherm followed by exotherm. The initial endotherm was similar to that observed from the supplied free form. HPLC data indicated that the material's purity remained high.

Weight loss in TGA was consistent with DSC large broad endotherm, which suggested possible hydrate behavior. Insufficient material was available to correlate the mass loss in the TGA with residual THE content by NMR. Chloride Pattern 1 remained stable by XRPD & HPLC after 7 days storage under 40° C./75% RH. IC results indicated Chloride Pattern 1 contained 0.5 mol equiv. of counter ion, suggesting incomplete salt formation. Based on the data available the sample was likely a mixture of a chloride salt and free form.

Sample 22-2 displayed an XRPD pattern consistent with phosphate pattern 1. The sample displayed high purity, the ¹H NMR was consistent with the expected structure, with <0.1 mol equiv. of residual THF present. The TGA showed a weight loss of 7 wt %, equivalent to 1.9 mol of water. A large broad endotherm (onset 82.1° C.) followed by a 2nd endotherm at 207° C. was visible in the DSC.

The weight loss in TGA was consistent with DSC large broad endotherm, which suggested possible hydrate behavior. Phosphate pattern 1 remained stable by XRPD & HPLC after 7 days storage under 40° C./75% RH. IC results indicate PHO Pattern 1 contained 1 mol equiv. of counter ion. Further analysis by PLM and HSM of PHO pattern 1 showed that sample consisted of thin needles, which remained stable up to 260° C., whereupon the sample began to melt.

Based on the data available the sample was likely a mono-phosphate hydrate. This salt form had reasonable properties, but with a large endotherm and mass loss at relatively low temperatures, the hydrate occupancy might be poorly defined at ambient conditions.

The results for the targeted screen of FUM Pattern 1 and MES Pattern 2 are summarized in Table 25.

TABLE 25
Characterization of Fumarate Pattern 2 and Mesylate Pattern 2

| Sample ID | 22-4 | 22-6 |
|---|---|---|
| XRPD (Wet) | FUM P2 | N/A |
| XRPD (Dry) | FUM P2 + ADD1 | MES P2 |
| ¹H NMR | Consistent with structure, ca. 0.6 | Consistent with structure, ca. 1.1 |
| (in d6-DMSO) | mol equiv. of fumarate detected. | mol equiv. of mesylate detected |
| | 0.2 mol equiv. of THF detected | |
| HPLC | 98.9% | 99.2% |
| (Generic method, | | |
| DMSO) | | |
| TGA | A weight loss of 14 wt % from 35 | A weight loss of 8 wt % from 35 to |
| | to 150° C. (equiv. to 3.5 mol of | 150° C. (equiv. to 2.2 mol of |
| | water and 0.2 mol of THF). | water). Decomposition above ca. |
| | Decomposition above ca. 250° C.* | 300° C. Poor quality |
| DSC | Large broad endotherm with an | Large broad endotherm with an |
| | onset of 41.3° C. (271 J/g). | onset of 65.0° C. (211 J/g). |
| | Followed by exotherm with an | Followed by double exotherm with |
| | onset of 146.6° C. (28 J/g) | a onset of 195.8° C. (42 J/g) |
| XRPD post | FUM P2 (additional peaks lost), | Unchanged, 99.1% |
| storage at 40° C./ | 99.2% | |
| 75% RH, 7 Days | | |
| Tentative | Hemi-fumarate hydrate. | Mono-mesylate hydrate |
| Assignment | | |

Key: ADD = Additional peaks
*For TGA, water mol equiv. calculation adjusted assuming 0.2 mol of THE present (as seen by NMR)

Sample 22-3 displayed an XRPD pattern which was not consistent with the previously obtained Fumarate Pattern 1 and as such was designated as a novel pattern (Fumarate Pattern 2). Furthermore, it was noted that on drying, the sample displayed additional peaks (FUM P2+ADD), previously unobserved while the sample was wet, suggested the form may be unstable. It also suggested there may be another fumarate form, accessed by drying Fumarate Form 2.

Analysis by ¹H NMR showed Fumarate Pattern 2 had a lower than anticipated stoichiometry, suggesting the formation of a hemi-salt. A small amount (0.2 mol equiv.) of residual THF was also noted. The sample remained high in purity.

The TGA showed a large weight loss of 14 wt %, equivalent to 3.5 mol of water and 0.2 mol of THF. A large broad endotherm (onset 41.3° C.), followed by an exotherm with an onset of 147° C. was visible in the DSC. The weight loss in TGA was consistent with DSC large broad endotherm, which suggested a possible hydrate.

Storage of the sample for 7 days under 40° C./75% RH resulted in the loss of the additional peaks observed after drying the material, which suggested Fumarate Pattern 2 is possibly a variable occupancy hydrate.

Due to the complex thermal behavior, combined with the apparent (partial) change in form, this salt form likely does not display ideal properties for use in a drug product.

Sample 22-6 displayed an XRPD pattern which was consistent with the previously obtained Mesylate Pattern 2.

Analysis by ¹H NMR showed the sample contained 1.1 mol equiv. of the mesylate counter ion with no residual solvent detected. HPLC showed the sample remained high in purity.

The TGA showed a weight loss of 8 wt % prior to decomposition (ca. 300° C.), equivalent to 2.2 mol of water. However, the data quality was poor and repeat measurement might be needed. The DSC thermogram showed a large broad endotherm (onset 65.0° C.) followed by a double exotherm with an onset of 196° C.

Storage of the sample for 7 days under 40° C./75% RH showed the material to be unchanged by XRPD or HPLC.

Based on the data available the sample was likely a mono-mesylate hydrate. As the dehydration of this material occurred close to ambient conditions, the solid form may not be suitable for further development as a drug product.

The results for the characterization of the material from the targeted screen on SUL Pattern 1 are summarized in Table 26.

TABLE 26
Characterization of Sulfate Pattern 1

| Sample ID | 22-8 |
|---|---|
| XRPD | SUL Pattern 1 + additional peaks |
| ¹H NMR | Consistent with structure, 1.9 mol equiv. of THF |
| (in d6-DMSO) | detected |
| HPLC (Generic | 99.3% |
| method, DMSO) | |
| TGA | A weight loss of 7 wt % from 35 to 150° C. |
| | (equiv. to 1.9 mol of water or 0.5 mol of THF). |
| | Decomposition above ca. 250° C. |
| DSC | Poorly defined asymmetrical endotherm with an |
| | onset of 36.3° C. (54 J/g) and a range of ca. 35 to |
| | 190° C. |
| PLM | Particulates, generally <25 μm in length which |
| | agglomerate. |
| XRPD post storage | SUL P2, 99.4% |
| at 40° C./75% | |
| RH, 7 Days | |
| Ion Chromatography | 0.7 mol equiv. of sulfate (15 wt %, adjusted for |
| (IC) | water content) |
| Tentative Assignment | Mixture of sulfate and free form |

Sample 22-8 displayed an XRPD pattern which was generally consistent with the previously obtained sulfate pattern 1, however additional peaks were noted.

HPLC showed the sample purity remained above 99%, IC analysis indicated the sample contained 0.7 mole equivalents of sulfate counter ion. PLM of the sample showed the particles to be <25 μm in size, and generally agglomerated. Analysis by ¹H NMR showed the sample was consistent with the supplied structure, with a high amount of residual solvent detected (1.9 mol equiv. of THF). TGA analysis showed a weight loss of 7 wt % prior to decomposition (ca. 250° C.), equivalent to 1.9 mol of water or 0.5 mol of THF. The disparity in the NMR and TGA data may indicate the sample was still wet when analyzed by NMR. The DSC thermogram showed a poorly defined, asymmetrical endotherm (onset 36° C.) with a large range (35 to 190° C.). A clear melt could not be observed.

Storage of the sample for 7 days under 40° C./75% RH showed the material to be unstable, transforming to a new pattern, denoted Sulfate Pattern 2. This new form shared many of the peaks with Sulfate Pattern 1 but appeared to be more crystalline. The sample purity remained high under storage.

In summary, a targeted salt screen was completed using Lot B of Structure (I). Conditions favorable for chloride, mesylate, phosphate, fumarate and sulfate salt formation were tested.

From the screen, Chloride Pattern 1, Phosphate Pattern 1, Mesylate pattern 2, Sulfate Pattern 1 (with additional peaks) and a novel Fumarate Pattern 2 were obtained. All patterns obtained were analyzed using a range of techniques. From the data obtained, samples generally appeared to be hydrated salts, with the possible exceptions of Chloride Pattern 1 and Sulfate Pattern 1, which appeared to be a mixture of the free form and salt form. The hydrated forms all lost the water readily at close to ambient conditions.

Based on these results, none of the salts stood out as suitable for further development as a drug product.

### Example 9

### Salt Screening Conclusions

Several experiments were performed to isolate salt forms of the compound of Structure (I). This has taken the form of a solubility and salt formation assessment and four salt screens—a standard screen with 12 common acids, a high temperature screen, a double equivalents screen and a targeted screen using high purity input material.

During this investigation, 12 salt forms were identified, along with two free base forms and some forms which were not unequivocally assigned. Some salts formed exhibited properties that were not desirable for use as a drug product. Several of the most promising candidates (e.g., PHO Pattern 1, MES Pattern 1) were characterized as hydrates, however, the water within these solids was lost at temperatures close to ambient conditions, which may result in lower stability than desired.

Also noted was a number of solids isolated with sub-stoichiometric amounts of counter ion (HCl Pattern 1) and a number of free form patterns were observed. Finally, two new free base forms, Free Form Pattern 2 and Free Form Pattern 3 were identified. Investigation of the polymorphic behavior of the free base was carried out and is presented in Examples disclosed herein.

### Example 10

### Polymorphism Screen of Structure (I) Free Form

As none of the identified salt forms were optimal, the free form of Structure (I) was further investigated. The study aimed to identify polymorphs of Structure (I), characterize them and determine their suitability for use in the drug product.

In order to facilitate the formation and identification of the wide number of forms, amorphous material was used as the input for the screen. Several different methodologies were tested for generation of amorphous material, as described further below.

Structure (I) (from Lot B, 30 mg, Free Form Pattern 3) was treated with increasing aliquots of THF:water (7:3 v/v) or MeCN:water (1:2 v/v) up to 100 vol (3 mL) at 50° C. After 100 volumes, the samples were left at 50° C. for 1 hr.

Neither sample dissolved, therefore freeze drying was not completed, and amorphous material could not be generated using this method.

### Reverse Anti-Solvent Addition (DMSO/TBME)

The compound of Structure (I) (from Lot B, 30 mg) was treated with aliquots of DMSO (5 vol, 150 μL) and stirred at 50° C. until dissolution (total 40 vol, 1.2 mL). TBME (12 mL, 1:10 solvent: anti-solvent volume ratio) was stirred at RT and the warm DMSO solution added dropwise to the TBME. The resulting suspension was filtered (Sample 27-1).

The suspension was characterized, and a summary of the data is presented in Table 27. The solid, Pattern 4, was a DMSO solvate, as evidenced by the large mass loss in the TGA and solvent content in the ¹H NMR spectrum. It converted to Pattern 3 on storage at 40° C./75% RH for 7 days.

TABLE 27
Characterization of Pattern 4

| Sample ID | 27-1 |
|---|---|
| XRPD | Crystalline—Pattern 4 |
| ¹H NMR | Trace TBME |
| | ~2.4 eq DMSO |
| TGA | 19.3 wt % loss 45-175° C. |
| | (~1.1 eq DMSO) |
| DSC | Broad double endotherm onset 37.3° C. (146 and 48 J/g) |
| | Double endotherm onset 165.1° C. (82 and 2 J/g) |
| Storage at 40° C./ | Change in form—Pattern 3 |
| 75% RH | |
| (7 days) | |
| Tentative | DMSO solvate |
| Assignment | |

### Reverse Anti-Solvent Addition (DMSO/Water)

The compound of Structure (I) (from Lot B, 30 mg) was dissolved in DMSO (40 vol, 1.2 mL). Water (12 mL, 1:10 solvent: anti-solvent volume ratio) was stirred at RT and the warm DMSO solution added dropwise to the water. The resulting suspension was filtered. XRPD analysis of the sample showed it to be Free Form Pattern 1.

### Ball Milling Test

Structure (I) (from Lot B, 30 mg) was added to a stainless-steel grinding jar with grinding ball. The sample was ground for 60 min at 30 Hz. Sample ID: 28-1

XRPD analysis of the material showed it to be amorphous other than two small peaks which were attributed to residual potassium chloride. The chemical purity did not deteriorate during the amorphization. The Tg was not determined by mDSC, as it appeared to be hidden under a large endotherm, indicating possible water loss. In the mDSC, two endothermic events attributed to crystallization were observed at 149.9° C. and 196.8° C. The amorphous material converted to Free Form Pattern 3 after storage at 40° C./75% RH for 7 days.

TABLE 28
Characterization of amorphous Structure (I)

| Sample ID | 28-1 |
|---|---|
| XRPD | Amorphous with peaks at 28.2 and 40.4° |
| HPLC | 98.0% |
| mDSC | Tg hidden by endotherm likely caused by |
| | water loss |
| | Broad endotherm onset 17.0° C. (118 J/g) |
| | Exotherm onset 149.9° C. (40 J/g) |
| | Exotherm onset 196.8° C. (41 J/g) |
| Storage at 40° C./ | 7 days: Crystallization-Pattern 3 (WD) |
| 75% RH | |

WD = weak diffraction

### Ball Milling for Screen 1

Structure (I) (from Lot B, 700 mg) was added to a 5 mL stainless steel grinding jar with a 9 mm grinding ball. The sample was ground for 60 min at 30 Hz. The sample was ground for a further 90 mins. XRPD analysis of the material showed that although crystallinity had been reduced, complete amorphization had not been achieved. This material (Sample 28-2) was used as input for the first screen.

### Ball Milling for Screen 2

The compound of Structure (I) (from Lot B, 1 g) was added to a 10 mL stainless steel grinding jar with a 9 mm grinding ball. The sample was ground for 90 min at 30 Hz. The sample was ground for a further 2×90 mins. Sample ID: 29-1

The increased duration of grinding and refined parameters allowed access to fully amorphous material. Characterization was consistent to that collected for the small-scale Sample 28-1 and is summarized in Table 29. This was used as input material for the second screen.

TABLE 29
Characterization of amorphous material used in the second screen

| Sample ID | 29-1 |
|---|---|
| XRPD | Amorphous |
| HPLC | 98.1% |
| mDSC | Tg hidden by endotherm likely caused by water loss |
| | Broad endotherm onset 78.3° C. (7 J/g) |
| | Exotherm onset 146.9° C. (39 J/g) |
| | Exotherm onset 176.1° C. (29 J/g) |

### Example 11

### Polymorph Screen 1—Poorly Crystalline Pattern 3 Input

Structure (I) (Sample 28-2, 30 mg, Poorly Crystalline Pattern 3) was wetted with solvent (300 μL, 10 vol) according to Table 30 and put into a shaker at 50° C. for 6 days. The suspensions were isolated using filter cartridges and positive pressure. A XRPD pattern was collected for each sample. Those exhibiting novel patterns were characterized further, including re-analysis by XRPD after leaving the samples to dry under ambient conditions overnight. This characterization is presented and discussed in Examples detailed herein. (see, e.g., FIG. 75)

The use of poorly crystalline Pattern 3 (Sample 28-2) as input material did not preclude the formation of 4 novel patterns. Patterns 5-9 all have XRPD patterns which are similar with some shifting of certain peaks, suggesting that they are structurally related forms, likely solvates. Pattern 8 and Pattern 9 were unstable to drying under ambient conditions, converting to Pattern 8 with peaks of Pattern 3 emerging.

TABLE 30
Results of polymorphism screening with poorly crystalline Pattern 3

| | | Observation | | |
|---|---|---|---|---|
| | | after 6 days | | |
| Sample | | maturation | | XRPD |
| ID | Solvent | at 50° C. | XRPD | (Dry) |
| 30-1 | n-Heptane | Susp. | Pattern 3 | — |
| 30-2 | Ethyl acetate | Thick Susp. | Pattern 5 | P5 + P3 |
| 30-3 | Isopropyl acetate | Susp. | P5 + P3 | P3 + P5 |
| 30-4 | MIBK | Susp. | Pattern 6 | Pattern 6 |
| 30-5 | 2-Propanol | Thick Susp. | Pattern 3 | — |
| 30-6 | MEK | Thick Susp. | Pattern 7 | Pattern 7 |
| 30-7 | Acetone | Thick Susp. | Pattern 3 + | — |
| | | | peak at 8.3° C. | |
| 30-8 | Ethanol | Thick Susp. | Pattern 8 | P8 + P3 |
| 30-9 | TBME | Thick Susp. | Pattern 3 (WD) | — |
| 30-10 | 2-Me-1-PrOH | Thick Susp. | Pattern 3 | — |
| 30-11 | Cyclohexane | Susp. | Pattern 3 | — |
| 30-12 | Methanol | Thick Susp. | Pattern 9 | P8 + P3 |
| 30-13 | Toluene | Thick Susp. | Pattern 3 | — |
| 30-14 | Tetrahydrofuran | Thick Susp. | Pattern 3 | — |
| 30-15 | Acetonitrile | Susp. | Pattern 8 | P8 + P3 |
| 30-16 | 10% Water/ | Thick Susp. | Pattern 3 | — |
| | EtOH | | | |
| 30-17 | 10% Water/IPA | Thick Susp. | Pattern 3 | — |
| 30-18 | 10% Water/THF | Susp. | Pattern 3 | — |

WD = weak diffraction

### Example 12

### Polymorph Screen 2—Amorphous Input

Amorphous Structure (I) (Sample 29-1, 30 mg) was wetted with solvent (300 μL, 10 vol) according to Table 31 and put into a shaker at 50° C. for 3 days. The samples were isolated using filter cartridges and frits and XRPD patterns collected. See, e.g., FIG. 77.

When using amorphous material as input for the screen, a less diverse result was obtained, with all but 4 of the samples yielding Pattern 3. Pattern 7 was isolated from MEK and ethanol, whereas these solvents yielded Pattern 7 and Pattern 8 in the first screen, respectively. Pattern 8 was obtained from MeOH and ACN, whereas these solvents yielded Pattern 9 (transforming to P8+P3 on drying) and Pattern 8 in the first screen, respectively.

TABLE 31
Results of polymorphism screening with amorphous material

| | | Observation after 3 days | |
|---|---|---|---|
| Sample ID | Solvent | maturation at 50° C. | XRPD |
| 31-1 | n-Heptane | Susp. | Pattern 3 |
| 31-2 | Ethyl acetate | Thick Susp. | Pattern 3 |
| 31-3 | Isopropyl acetate | Susp. | Pattern 3 |
| 31-4 | MIBK | Susp. | Pattern 3 |
| 31-5 | 2-Propanol | Thick Susp. | Pattern 3 |
| 31-6 | MEK | Thick Susp. | Pattern 7 |
| 31-7 | Acetone | Thick Susp. | Pattern 3 |
| 31-8 | Ethanol | Thick Susp. | Pattern 7 |
| 31-9 | TBME | Thick Susp. | Pattern 3 |
| 31-10 | 2-Me-1-PrOH | Thick Susp. | Pattern 3 |
| 31-11 | Cyclohexane | Susp. | Pattern 3 |
| 31-12 | Methanol | Thick Susp. | Pattern 8 |
| 31-13 | Toluene | Thick Susp. | Pattern 3 |
| 31-14 | Tetrahydrofuran | Thick Susp. | Pattern 3 |
| 31-15 | Acetonitrile | Susp. | Pattern 8 |
| 31-16 | 10% Water/EtOH | Thick Susp. | Pattern 3 |
| 31-17 | 10% Water/IPA | Thick Susp. | Pattern 3 |
| 31-18 | 10% Water/THF | Susp. | Pattern 3 |

Novel forms identified in the polymorph screens were characterized to determine the nature of the solid forms.

TABLE 32
Characterization of new forms, Pattern 5 + 3 and Pattern 6

| Sample ID | 30-2 | 30-4 |
|---|---|---|
| XRPD | Crystalline-Pattern 5 and 3 | Crystalline- |
| | | Pattern 6 |
| ¹H NMR | ~0.2 eq ethyl acetate | ~0.5 eq MIBK |
| TGA | 0.2 wt % loss 40-65° C. | 12.3 wt % loss |
| | 1.6 wt % loss 65-120° C. | 95-205° C. |
| | 4.6 wt % loss 120-200° C. | (~0.5 eq MIBK) |
| | (Total weight loss ~0.3 eq | |
| | ethyl acetate) | |
| DSC | Broad endotherm onset 89.6° C. | Endotherm onset |
| | (27 J/g) | 167.9° C. (55 J/g) |
| | Endotherm onset 156.1° C. (1 J/g) | |
| | Endotherm onset 178.7° C. (7 J/g) | |
| | Exotherm onset 193.5° C. (28 J/g) | |
| Storage at | 7 days: Change in form-Pattern 3 | 7 days: Change in |
| 40° C./75% RH | | form-Pattern 3 |
| Tentative | Acetate (ethyl and isopropyl) | MIBK solvate |
| Assignment | solvate | |

Sample 30-2 was originally isolated as Pattern 5 but on mild drying under ambient conditions started to convert to Pattern 3. This transition completed under storage at 40° C./75% RH for 7 days. The NMR showed 0.2 mole equivalents of ethyl acetate were present in the sample, which was in reasonable agreement with the TGA. Along with endotherms associated with solvent loss, the DSC also contained and endotherm after the mass loss starting at ca. 180° C.

Sample 30-4 was isolated as Pattern 6, which was maintained after mild drying, however, did convert to Pattern 3 when stored at 40° C./75% RH for 7 days. The NMR and TGA both suggested that there were 0.5 mole equivalents of MIBK present in the sample, suggesting that the sample is a MIBK solvate.

TABLE 33
Characterization of new forms, Pattern 7 and Pattern 8 + 3

| Sample ID | 30-6 | 30-15 |
|---|---|---|
| XRPD | Crystalline-Pattern 7 | Crystalline-Pattern 8 and 3 |
| ¹H NMR | ~0.4 eq MEK | No residual solvents |
| TGA | 0.4 wt % loss 40-85° C. | 3.6 wt % loss 40-145° C. |
| | 7.6 wt % loss 85-195° C. | (~0.8 eq water) |
| | (Total weight loss ~0.4 eq MEK) | |
| DSC | Broad endotherm onset 32.9° C. (7 J/g) | Broad double endotherm onset |
| | Endotherm onset 141.6° C. (3 J/g) | 35.8° C. (90 and 10 J/g) |
| | Endotherm onset 173.8° C. (32 J/g) | Triple exotherm onset |
| | Exotherm onset 199.9° C. (23 J/g) | 146.6° C. (11, 11 and 45 J/g) |
| Storage at | 7 days: Change in form-Pattern 3 | 7 days: Change in form- |
| 40° C./75% RH | | Pattern 3 |
| Tentative | MEK solvate | Hydrate |
| Assignment | | |

Sample 30-6 was isolated from MEK and from the XRPD has been denoted Pattern 7. The TGA and ¹H NMR are in agreement with a solvent content of 0.4 mole equivalents. The material was not stable to static storage at 40° C./75% RH for 7 days, converting to Pattern 3.

Sample 30-15 was denoted as Pattern 8+3 having converted under ambient drying for 1 day from Pattern 9. Interestingly the NMR showed no residual solvent, but the TGA contained a mass loss of 3.6% between 40-145° C. suggesting there were 0.8 mole equivalents of water present in the material.

### Example 13

### Generation of Pattern 7 from Crystalline Pattern 3

Pattern 7, a solvate form, was obtained from MEK in both screens. The input for these screens was either amorphous or poorly crystalline Structure (I). This experiment was conducted to determine whether the solvate could be formed under the same conditions using crystalline Pattern 3 as the input material.

Structure (I) (from Lot B, 30 mg) was wetted with MEK (300 μL, 10 vol) and put into a shaker at 50° C. for 3 days. The sample was aliquoted for XRPD.

XRPD of the sample showed that Pattern 3 had converted to Pattern 7. This shows that the formation of the solvate was favored even when using the more stable crystalline material as input.

### Example 14A

### Generation of Pattern 11—Method A

Pattern 11 was first identified in the VT-XRPD of Pattern 3 (from Lot B) and was formed after dehydration of Pattern 3 to Pattern 10 and then high temperature conversion (at 250° C.) to Pattern 11. In the VT experiment, Pattern 11 was obtained as a mixture with Pattern 3. This experiment was conducted to ascertain if this form could be isolated as a phase pure material and whether it was stable under ambient conditions.

Structure (I) (from Lot B, 100 mg) was heated in an oven at 250° C. for 1 hour. After 1 hour, the oven was turned off but the sample kept inside to cool down. The sample was removed from the oven after 1 hour cooling and analyzed by XRPD (Sample 34-1).

The sample was found to be Pattern 11, without any other forms present. It was also found to be stable at ambient conditions, suggesting it could be a form suitable for further development.

TABLE 34
Characterization of Pattern 11

| ID | 34-1 |
|---|---|
| XRPD | Pattern 11 |
| TGA | 0.1 wt % loss 35-100° C. |
| | (<0.1 eq water) |
| DSC | No events up to 340° C. |
| XRPD after | Pattern 11 |
| ambient overnight | 3 days: No change in form-Pattern 11 |
| Storage at 40° C./ | 7 days: Pattern 11 + extra peaks (not |
| 75% RH | consistent with any other observed forms) |

### Example 14B

### Formation of Pattern 11—Method B

A polymorphism study of Structure (I) was performed at 75° C., 100° C., 175° C., and 250° C. About 500 mg of Structure (I) Pattern 3 was heated at 75° C. for 24 h in a vacuum tray dryer (VTD) under reduced pressure. Obtained material after heating at 75° C. was analyzed for purity by HPLC and XPRD analysis. Results showed the sample remained as Pattern 3 by XRPD and there was no change in HPLC purity. Additionally, about 500 mg of Structure (I) was heated at 75° C. for 24 h in a VTD under reduced pressure. After 24 h, material was cooled to 25-30° C. under inert atmosphere and analyzed for purity by HPLC and XRPD.

About 500 mg of Structure (I) was heated at 100° C. for 24 h in a VTD under reduced pressure. Obtained material after heating at 100° C. was analyzed for purity by HPLC and XPRD. The resulting analysis showed the sample remained as Pattern 3 by XRPD and showed no change in HPLC purity. Additionally, about 500 mg of Structure (I) was heated at 100° C. for 24 h in a VTD under reduced pressure. After 24 h material was cooled to 25-30° C. under inert atmosphere. Obtained material was analyzed for purity by HPLC and XPRD.

About 500 mg of Structure (I) was heated at 175° C. for 24 h in a VTD under reduced pressure. Obtained material after heating at 175° C. was analyzed for purity by HPLC and XPRD. The resulting analysis showed the sample was converted to Pattern 11 as confirmed by XRPD; no change to HPLC purity was detected. Additionally, about 500 mg of Structure (I) was heated at 175° C. for 24 h in a VTD under reduced pressure. After 24 h, material was cooled to 25-30° C. under inert atmosphere. Obtained material was analyzed for purity by HPLC and XPRD.

About 500 mg of Structure (I) was heated at 250° C. for 24 h in a VTD. Obtained material after heating at 250° C. was analyzed for purity by HPLC and XPRD. The resulting analysis showed the sample was converted to Pattern 11 as confirmed by XRPD. During sample preparation for HPLC analysis, the sample showed turbidity in the diluent (10% DMSO in MeOH) used for sample preparation.

Before proceeding for the heating experiment at 250° C., material was tested for DSC study. DSC results showed no primary safety issues were observed up to a temperature 400° C.

About 500 mg of Structure (I) was heated at 250° C. for 24 h in a VTD. After 24 h, the sample was cooled to 25-30° C. under inert atmosphere. Obtained material was analyzed for purity by HPLC and XPRD analysis.

Based on results obtained from 175° C. and 250° C. (i.e., where Pattern 11 was obtained and confirmed by XPRD analysis), a polymorphism study was repeated on 2 g scale to generate Pattern 11 seeding material at 175° C.

### Example 15

### Formation of Pattern 1—Ion Content Analysis

Pattern 1 has been observed to form during solubility analysis in pH 2 buffer and also pH 1.6 FaSSGF simulated fluid. It was hypothesized that the formation of Pattern 1 may indicate formation of an HCl salt. Pattern 1 was produced by slurrying Pattern 3 in pH 2 buffer.

Structure (I) (from Lot B, 30 mg) was suspended in pH 2.0 buffer (chloride buffer, 3 mL) and placed in a shaker at RT. After 1 day, the sample was filtered and analyzed by XRPD. The sample was re-suspended in pH 2.0 buffer (2 mL) and returned to shaking at RT for 4 days. The sample was aliquoted and analyzed by XRPD, before filtration and drying under suction. After 5 days, the sample was found to be Pattern 1.

Ion chromatography showed the presence of 0.49 mole equivalents of chloride. The sub-stoichiometric amount of chloride suggested that only partial salt formation occurred.

Pattern 1 was previously obtained from reverse anti-solvent addition with DMSO into water with no chloride present and was also the form of the input material from Lot A, which contained no chloride. Therefore, it is likely that if a chloride salt had been produced in the solubility measurements, that it was amorphous and not detected by XRPD. If this is the case, it can be concluded that the resulting solid is a mixture of free base Pattern 1 and amorphous HCl salt.

### Example 16

### Summary of Polymorphism Assessment

Two polymorph assessments were carried out on the compound of Structure (I) free base, one using poorly crystalline Pattern 3 (Sample 28-2) as an input material and the second using amorphous compound of Structure (I) as input material. During these screens Patterns 5-9 were identified, and Patterns 5-8 were characterized (Pattern 9 was unstable at ambient conditions). Pattern 4 was also identified during an attempt to generate amorphous material by reverse anti-solvent addition (DMSO into TBME). Finally Pattern 11, identified during the VT-XRPD of Pattern 3, was generated by heating Pattern 3 to 250° C. in an oven.

Pattern 4 was determined to be a DMSO solvate. Pattern 5-9 have similar XRPD diffractograms and are likely structurally related. Pattern 5, Pattern 6, and Pattern 7 were shown to be solvates of ethyl/isopropyl acetate, MIBK and MEK, respectively, suggesting that these are most likely a family of structurally similar solvates. Pattern 8 did not appear to have any solvent present, despite having a similar diffractogram, so it may also be possible that the void in these family of structures can be occupied by water. Due to the propensity of this structure to include solvent and its conversion to Pattern 3 under high temperature and humidity, these forms are likely not ideal for development as a drug product.

Pattern 11 appeared to be an anhydrous form with reasonable stability at 40° C./75% RH and so investigations were carried out to characterize this form further.

### Example 17

### Formation and Characterization of New Crystalline Forms

Structure (I) (from Lot B, 750 mg) was heated in an oven at 250° C. for 1.5 hours. After 1.5 hours, the oven was turned off but the sample kept inside to cool down. The sample was removed from the over after 1 hour cooling and analyzed by XRPD.

Sample 35-1 was characterized using a wide range of techniques to investigate the solid form properties of Structure (I) Pattern 11. A summary of the results is presented in Table 35.

TABLE 35
Characterization summary Pattern 11

| Sample ID | 35-1 |
|---|---|
| XRPD | Pattern 11 |
| ¹H NMR | Consistent with structure, no solvents |
| HPLC | 97.9% |
| PLM | Soft agglomerates of lath shaped particles up to |
| | 120 μm |
| TGA | No mass loss observed until degradation ~340° C. |
| DSC | No notable events up to 350° C. |
| | Slight change in baseline at 290° C. |
| GVS | 0.3 wt % reversible water uptake 0-90% RH with a |
| | small hysteresis. |
| | Post GVS XRPD: Pattern 11 + peak at 7.7° |
| | (Extra peak does not correspond to any known forms) |
| Static Storage | 10 days: No change in form-Pattern 11 |
| 40° C./75% RH | Purity: 98.0% |
| Static Storage | 10 days: No change in form-Pattern 11 |
| 25° C./97% RH | Purity: 98.1% |
| Solubility | FaSSGF-0.078 mg/mL |
| | FeSSIF-0.0040 mg/mL |
| | FaSSIF-0.0014 mg/mL |
| | pH 2.0-0.052 mg/mL |
| | pH 7.4-0.001 mg/mL |

After being generated in the oven at 250° C., the sample was found to be Pattern 11 by XRPD. The ¹H NMR was consistent with the structure and showed no evidence of residual solvent. The measured purity of the material was found to be 97.9%, which suggested that no significant degradation had occurred despite the high temperatures involved in the generation of the form (input material purity=98.1%). PLM of the material showed it to be comprised of soft agglomerates of lath shaped crystals up to 120 μm in length. The crystals were fractured and were not suitable for analysis by single crystal X-ray diffraction.

The TGA showed no mass loss before the onset of decomposition at >300° C., suggesting it is an anhydrous form. The DSC had no clear events other than a possible change in baseline at 290° C., no melt was observed up to 350° C. GVS showed that the Pattern 11 was slightly hygroscopic, exhibiting a very slight hysteresis in the isotherm. The sample remained Pattern 11 after the double-cycle experiment with an extra peak observed at 7.7° 20. The sample was stored at elevated temperature and/or humidity for 10 days and the form and purity was found to stable.

The thermodynamic solubility for Pattern 11 was determined in 3 simulated media and two buffers. Pattern 11 was found to be practically insoluble in the media, showing the highest solubility in the low pH media.

The characterization data for Structure (I) Pattern 11 suggested that it is a solid form that would be suitable for use in a drug product. It is an anhydrous form that is stable under high humidity conditions and is only slightly hygroscopic.

### Example 18

### Competitive Slurries and Stability Relationships

A solid mixture of Structure (I) Pattern 3 (from Lot B, 300 mg) and Pattern 11 (Sample 35-1, 300 mg) was mixed for 3 hours using a turbula mixer.

The solid mixture (30 mg) was suspended in solvent (dried with molecular sieves, 600 μL, 20 vol) and either stirred in the fridge, shaken at RT or 55° C. After 7 days, an aliquot of each suspension was analyzed by XRPD. Samples that were still a mixture of forms were placed back into fridge/shakers for a further 10 days and re-analyzed (results shown in Table 36).

The results of the competitive slurries were inconclusive as at no temperature did one form persist over the other in all solvent systems. In neat THF and IPA, Pattern 11 was obtained after slurring for 7 days at all temperatures, however, in the case of IPA, additional peaks were present in the XRPD, which matched those that are associated with the group of solvates represented by Patterns 5-9, suggesting that conversion could have proceeded via a solvated form and thus cannot inform the relative stability of Pattern 3 and Pattern 11. Where conversion was observed, Pattern 3 converted to Pattern 11, but no conversion of Pattern 11 to Pattern 3 was seen.

TABLE 36
Results and observation of the competitive slurry experiments

| Sample | | | Observation | XRPD after 7 | XRPD after 17 |
|---|---|---|---|---|---|
| ID | Solvent | Temp. | after 7 days | days | days |
| 36-1 | IPA | 5° C. | White suspension | Pattern 11 | — |
| 36-2 | THF | (Fridge) | White suspension | Pattern 11 | — |
| 36-3 | Toluene | | White suspension | Patterns 3 and 11 | Patterns 3 and 11 |
| 36-4 | IPA/Water | | White suspension | Pattern 11 | — |
| | (95:5) | | | | |
| 36-5 | THF/Water | | White suspension | Pattern 11 | — |
| | (9:1) | | | | |
| 36-6 | IPA | RT | Cream suspension | Pattern 11 | — |
| 36-7 | THF | | Cream suspension | Pattern 11 | — |
| 36-8 | Toluene | | Cream suspension | Patterns 3 and 11 | Patterns 3 and 11 |
| 36-9 | IPA/Water | | Cream suspension | Patterns 3 and 11 | Patterns 3 and 11 |
| | (95:5) | | | | |
| 36-10 | THF/Water | | Cream suspension | Patterns 3 and 11 | Patterns 3 and 11 |
| | (9:1) | | | | |
| 36-11 | IPA | 55° C. | Cream suspension | Pattern 11 + extra | — |
| | | | | peaks* | |
| 36-12 | THF | | Cream suspension | Pattern 11 | — |
| 36-13 | Toluene | | Cream suspension | Patterns 3 and 11 | Patterns 3 and 11 |
| 36-14 | IPA/Water | | Cream suspension | Patterns 3 and 11 | Patterns 3 and 11 |
| | (95:5) | | | | |
| 36-15 | THF/Water | | Cream suspension | Patterns 3 and 11 | Pattern 11 |
| | (9:1) | | | | |

*extra peak matches Pattern 9

In order to determine that Pattern 3 was not simply being dissolved in the solvent systems when the solid was introduced, gravimetric solubility measurements were made using the samples at room temperature. These values are presented in Table 37. It can be seen that the solubility was highest in THF:water 9:1 at 8.1 mg/mL. The amount of Pattern 3 in the solid mixture introduced into each competitive slurry (0.6 mL) was 15 mg, thus a solubility of 25 mg/mL would be required to dissolve all of the Pattern 3. There was insufficient solubility at room temperature to dissolve the Pattern 3 present in the competitive slurry experiments.

TABLE 37
Gravimetric solubility measurements from competitive slurry
samples^{†}

| | | | Gravimetric |
|---|---|---|---|
| Sample | | XRPD after | solubility |
| ID | Solvent | 17 days | (mg/mL) |
| 36-6 | IPA | — | *Inconclusive |
| 36-7 | THF | — | *Inconclusive |
| 36-8 | Toluene | Patterns 3 and 11 | 2.3 |
| 36-9 | IPA/Water (95:5) | Patterns 3 and 11 | 3.8 |
| 36-10 | THF/Water (9:1) | Patterns 3 and 11 | 8.1 |

*Inconclusive values due to low mass of solid in vials (negative mass recorded)
^{†}Measurements were made at room temperature

Competitive slurry experiments at 55° C., along with one at 5° C., were repeated using a saturated solution as the solvent mixture to minimize dissolution that may have occurred at temperatures other than room temperature.

A saturated solution of THF/Water (9:1) (5 mL) with Structure (I) (from Lot B) was equilibrated at 55° C. overnight in a shaker. The solution was filtered using 0.45 μm nylon filter before use.

A solid mixture of Pattern 3 and Pattern 11 (30 mg) was suspended in the saturated solution (600 μL, 20 vol) and either stirred in the fridge (5° C.) or shaken at 55° C. After 1 day, aliquots of the suspensions were analyzed by XRPD. Both samples were placed back into fridge/shaker. After a total of 11 days, the samples were removed from fridge/shaker. Sample 38-2 was filtered before XRPD, Sample 38-1 was pipetted onto the XRPD holder and allowed to dry before analysis (solid passed through filter).

The results of this experiment using saturated solutions as the slurry media are summarized in Table 38. Both experiments resulted in mixtures of Pattern 3 and 11 with no notable intensity change. This is in contradiction to the first competitive slurry experiments, where both of these samples yielded Pattern 11. The difference in the result of the experiment at 55° C. might be due to the dissolution of the Pattern 3 in the first experiment (Sample 36-15), which did not occur in this experiment (Sample 38-2). In conclusion, the series of competitive slurries have not unequivocally shown that Pattern 3 and Pattern 11 interconvert, or one form is more stable than the other.

TABLE 38
Results of additional competitive slurry experiments

| Sample | | | XRPD after 1 | Observation after | XRPD after 11 |
|---|---|---|---|---|---|
| ID | Solvent | Temp | day* | 1 and 11 days | days |
| 38-1 | THF/Water | 5° C. | Patterns 3 and 11 + | White suspension | Patterns 3 and |
| | (9:1) | (Fridge) | peak at 6.7° | | 11 |
| 38-2 | THF/Water | 55° C. | Patterns 3 and 11 + | Cream suspension | Patterns 3 and |
| | (9:1) | | peak at 7.1° | | 11 |

*The origin of the additional peaks seen at the one-day time point is unknown, as these do not correlate to any previously observed form.

### Example 19

### Summary of Conditions for Obtaining Solid Forms of Pattern 1-11

A polymorphism assessment was carried out of the free form of the compound of Structure (I). This assessment involved an investigation into the preparation of amorphous material followed by two screens. The first utilized poorly crystalline Pattern 3 of the compound of Structure (I) as the input material, whilst the second used amorphous compound of Structure (I).

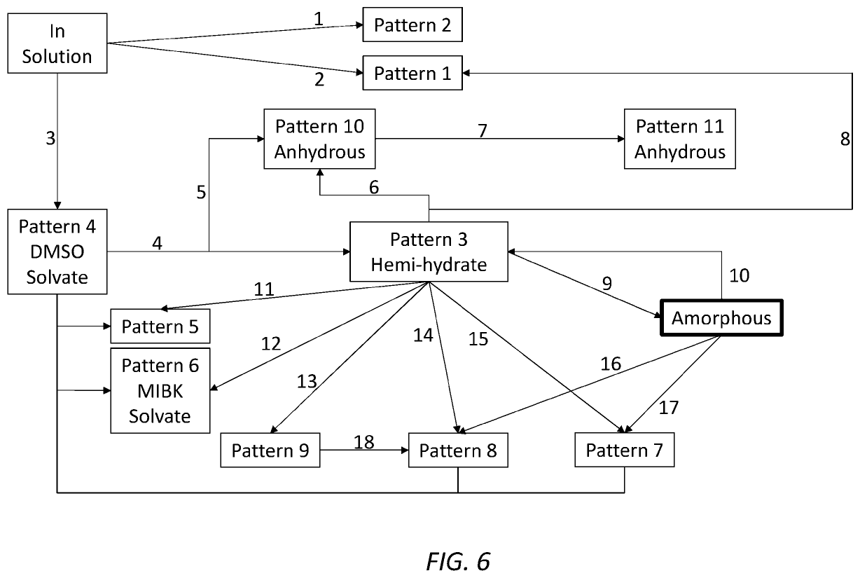

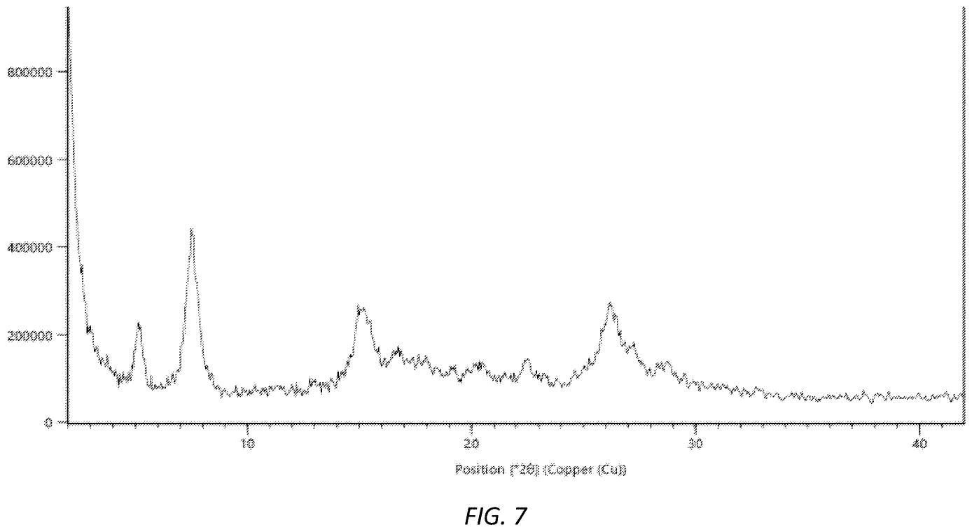

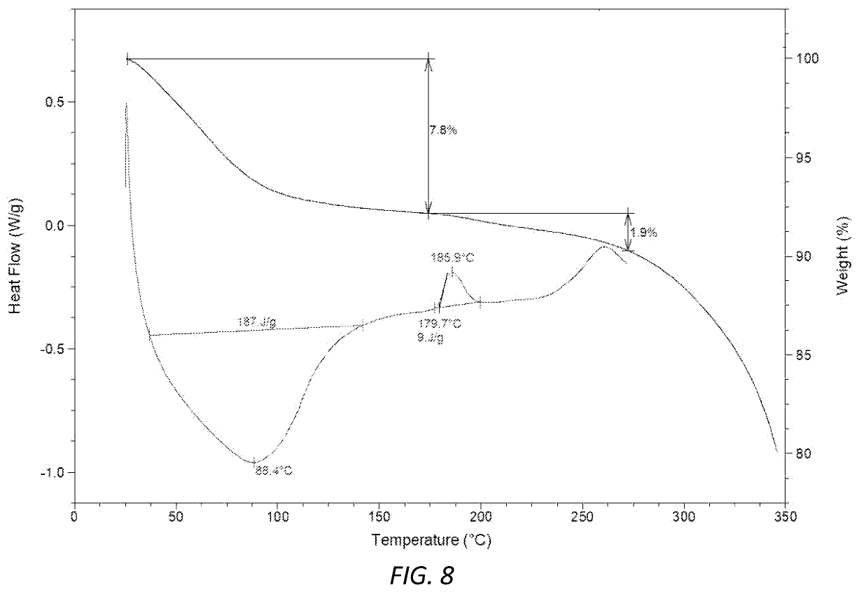

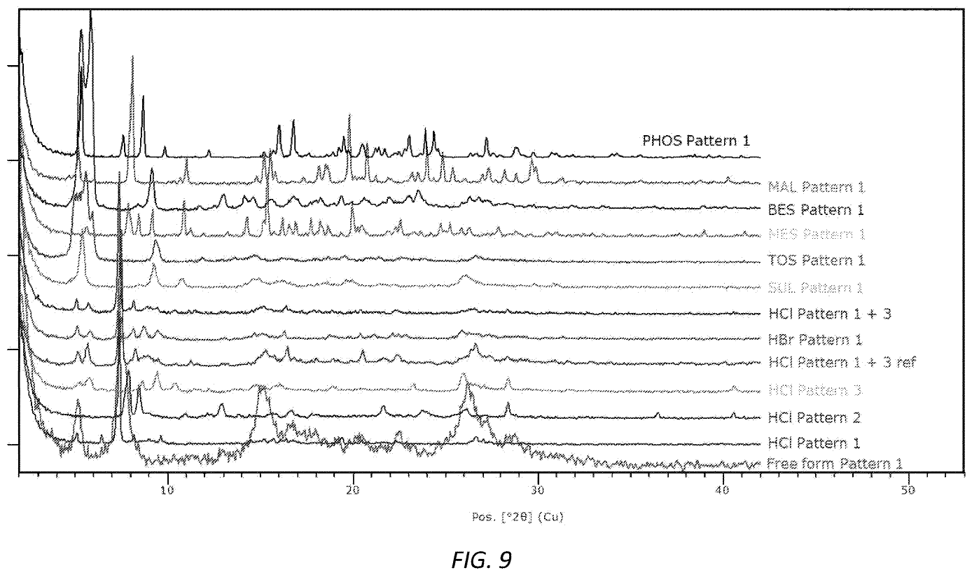

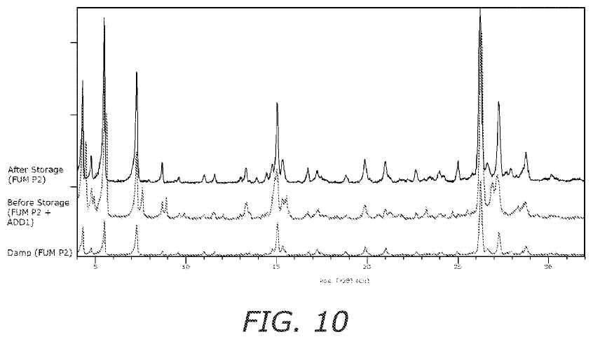

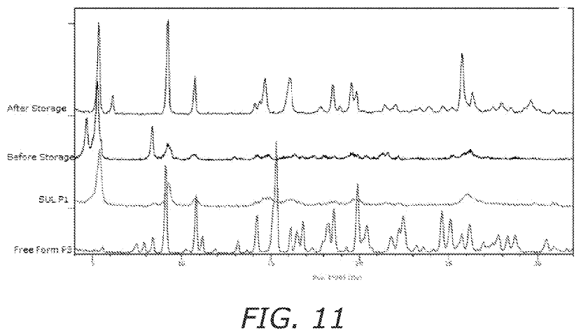

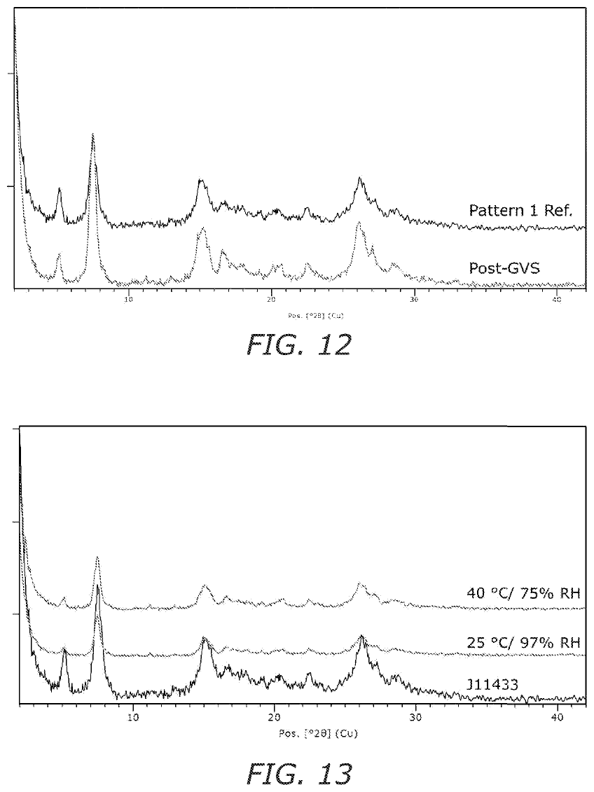

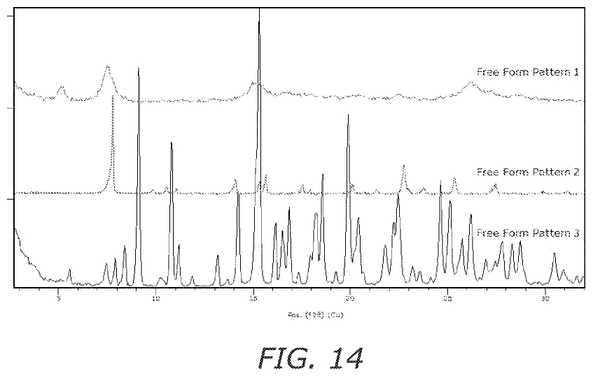

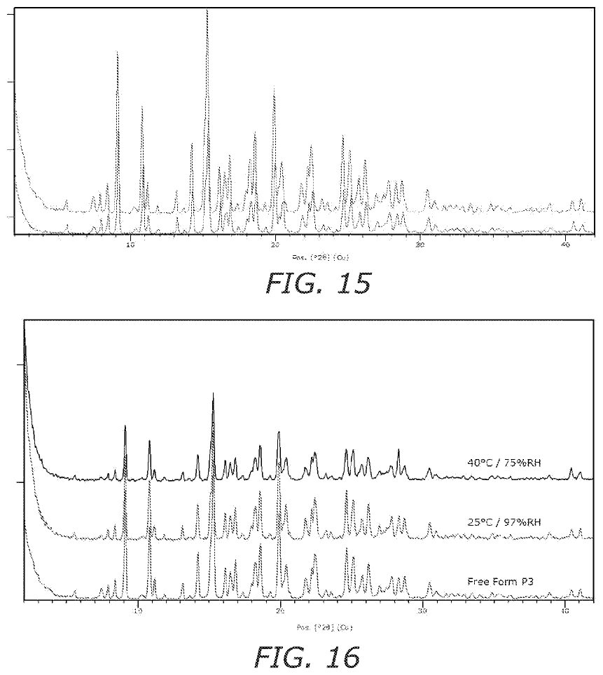

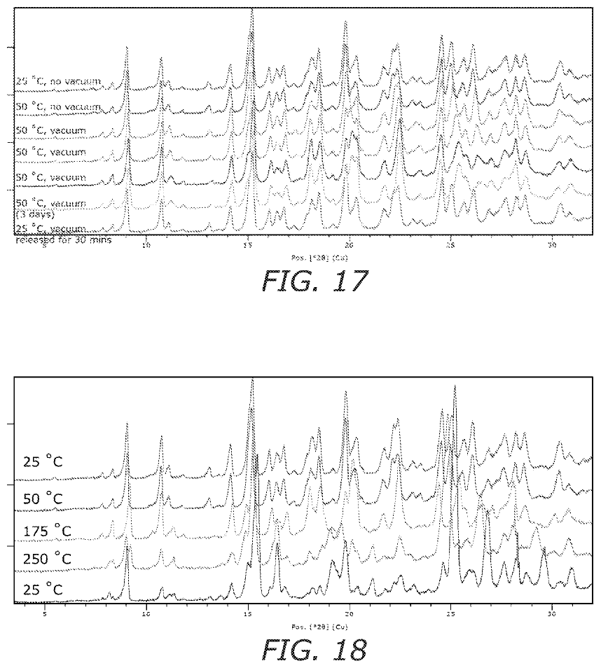

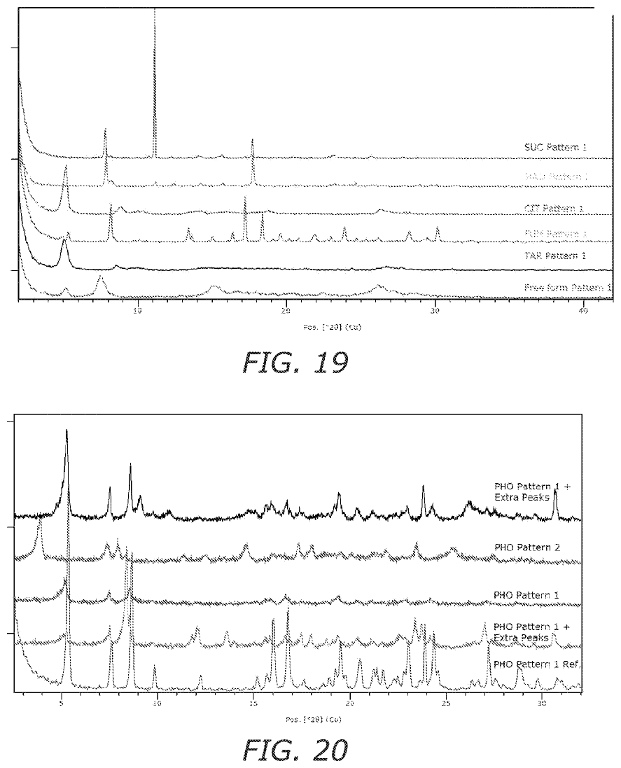

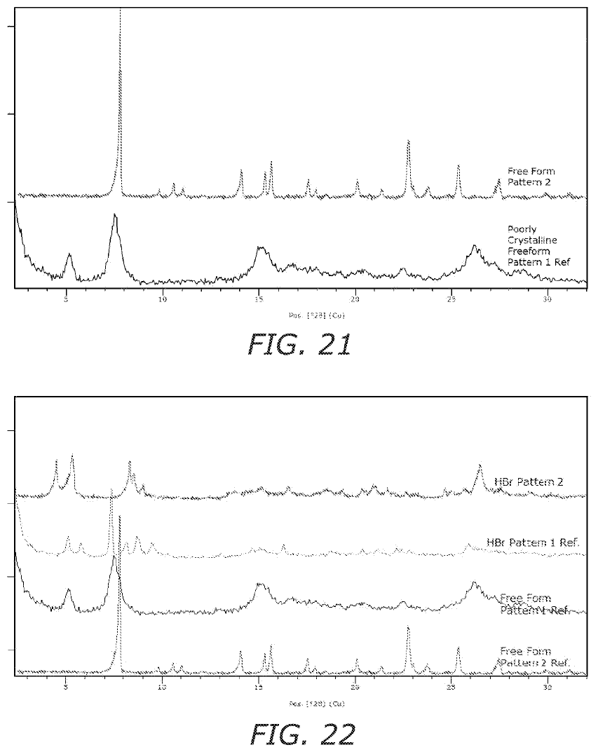

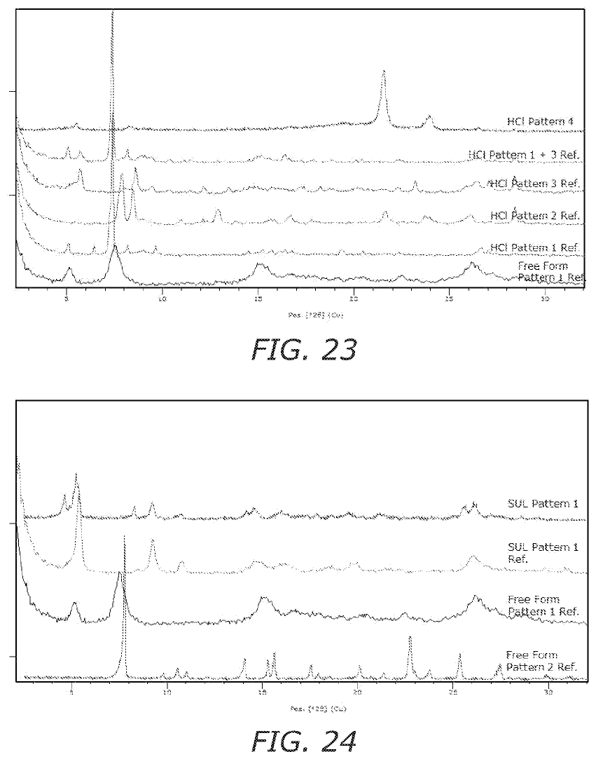

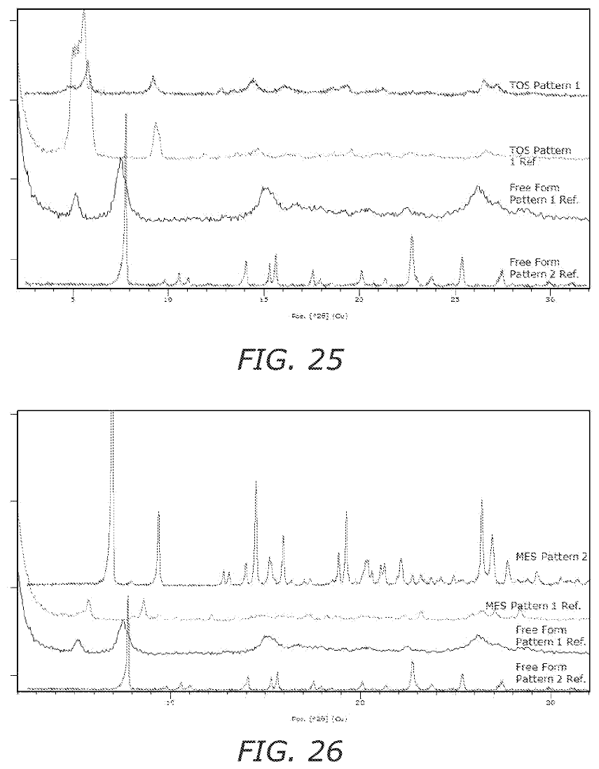

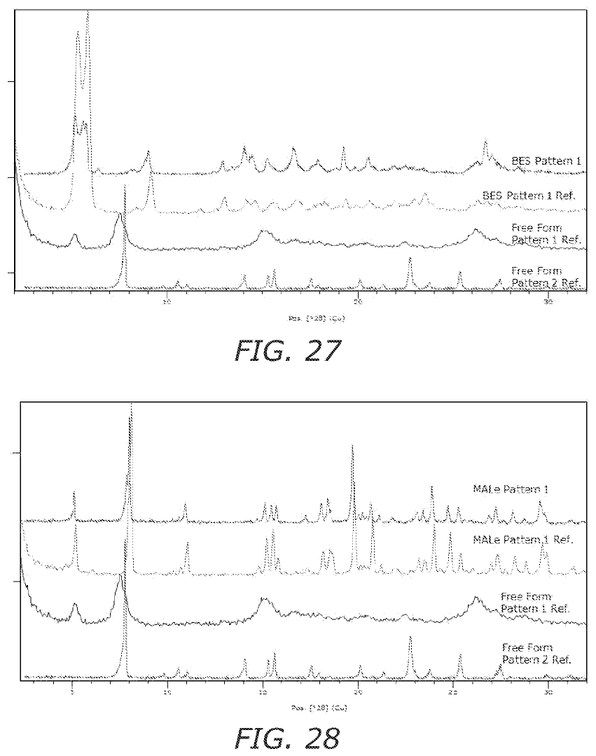

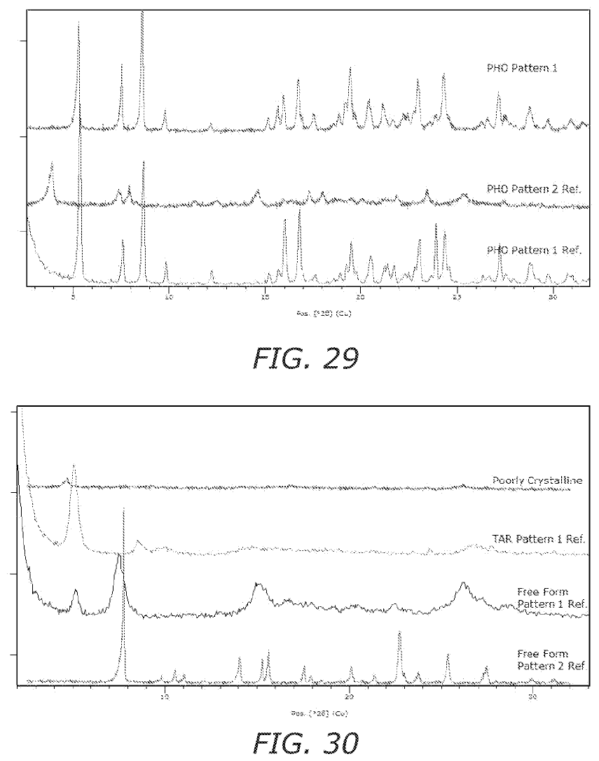

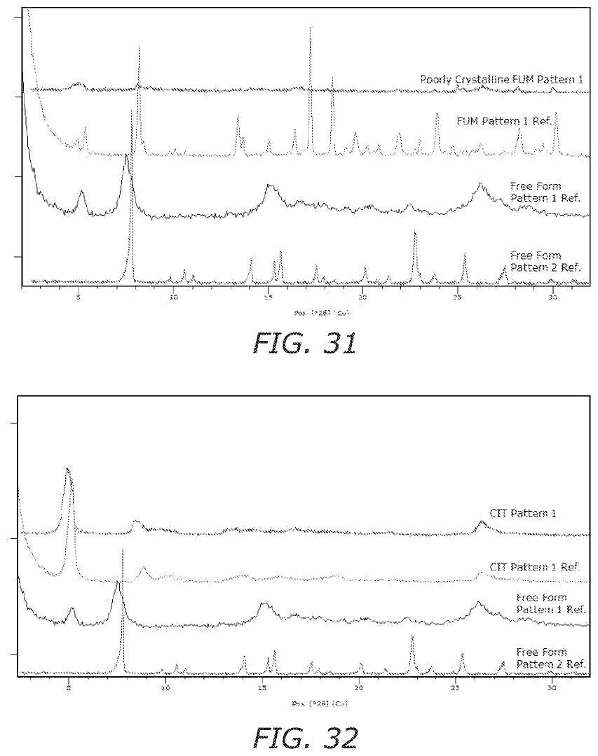

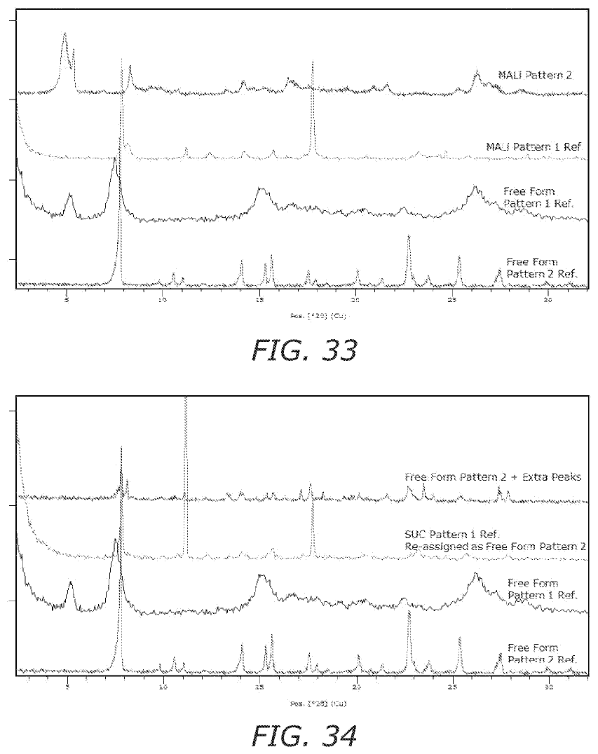

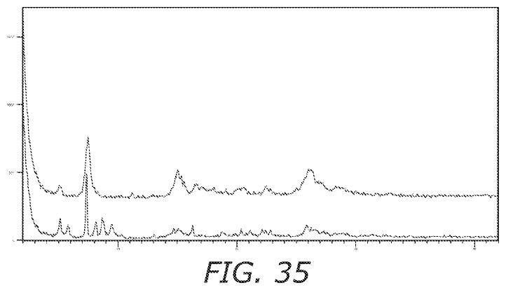

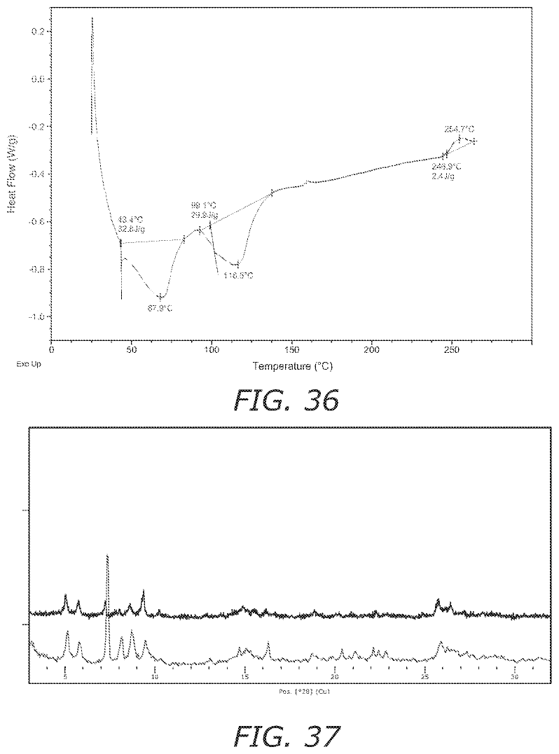

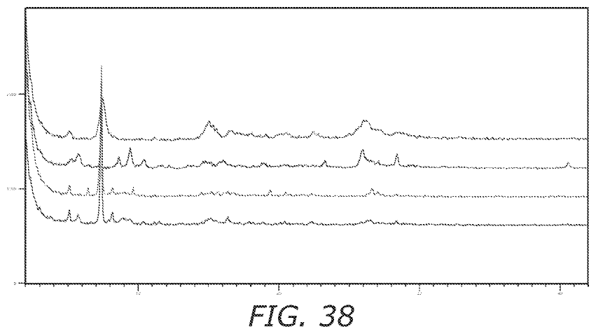

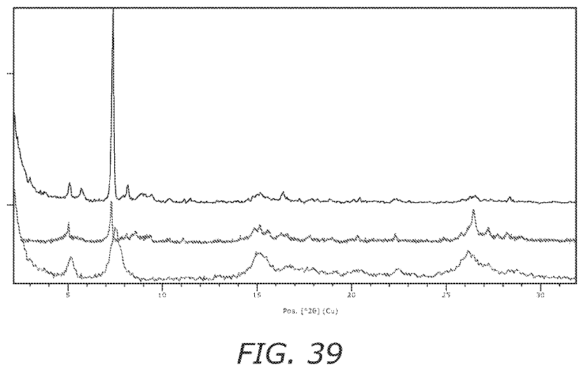

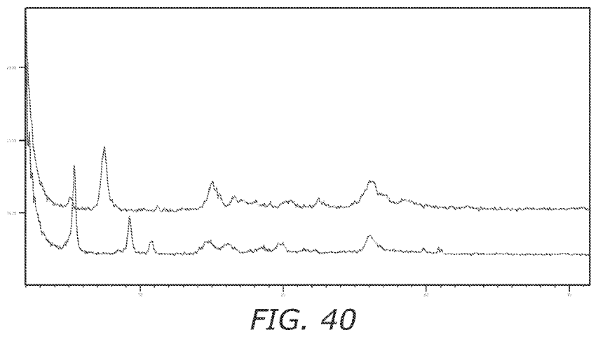

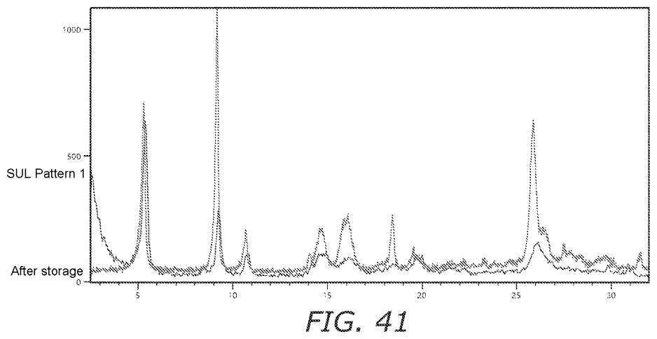

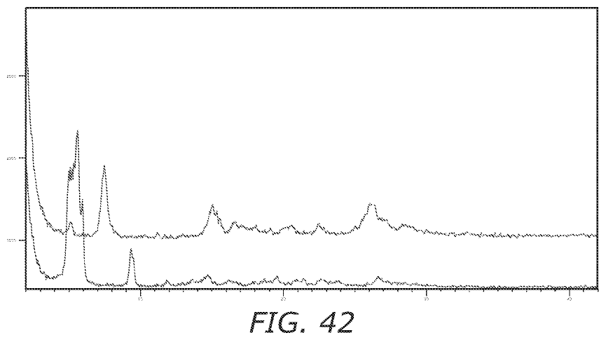

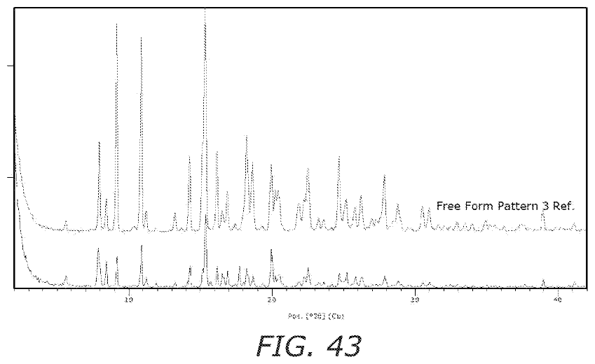

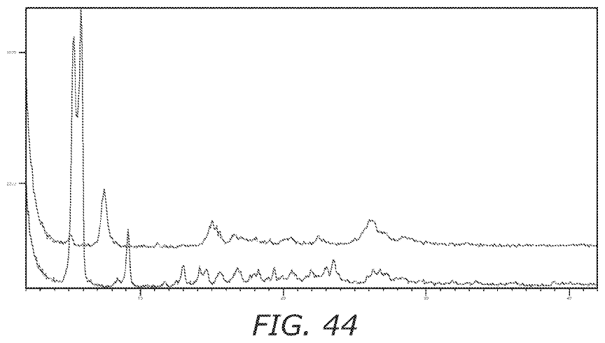

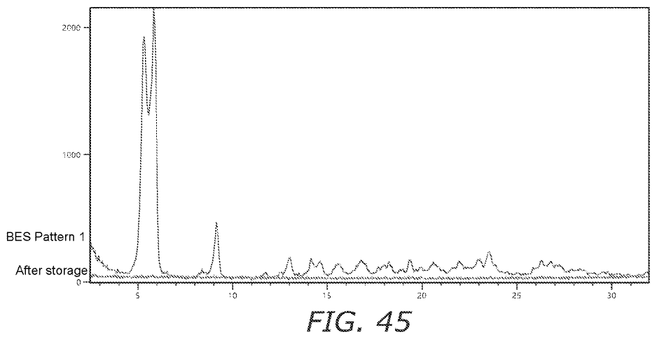

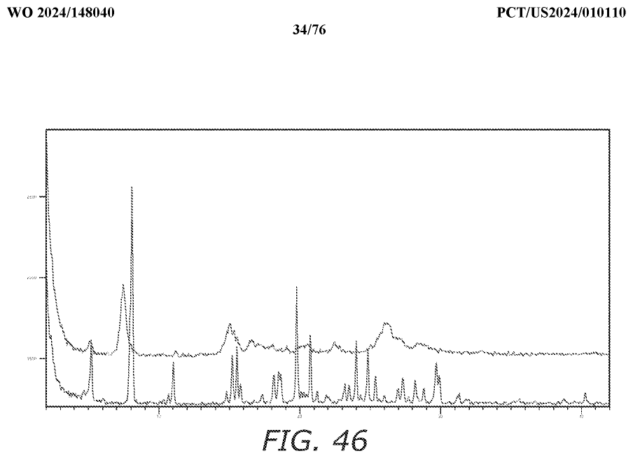

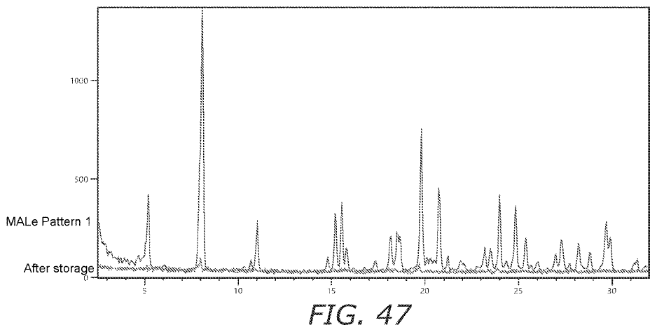

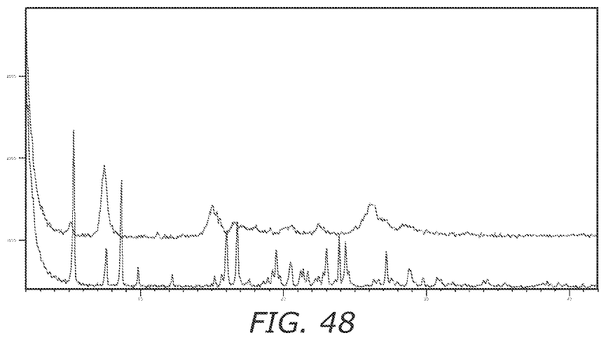

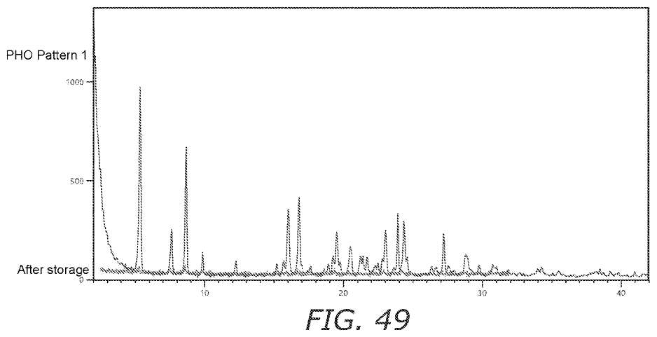

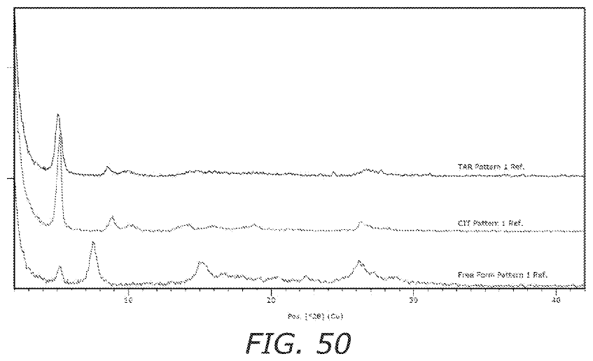

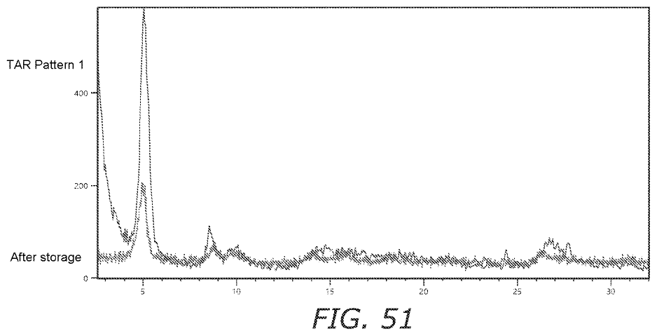

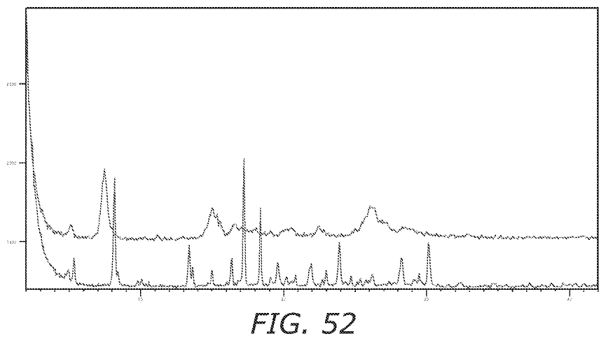

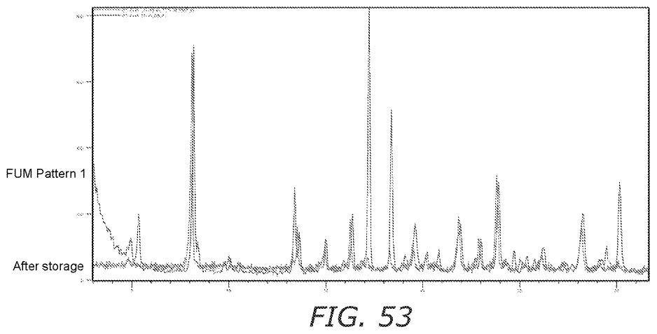

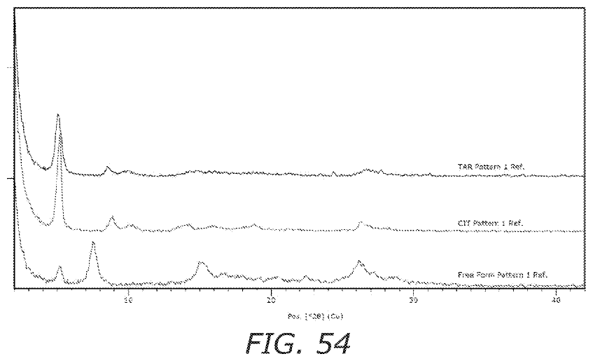

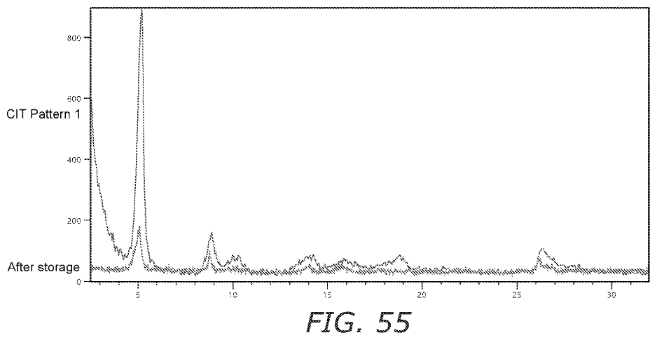

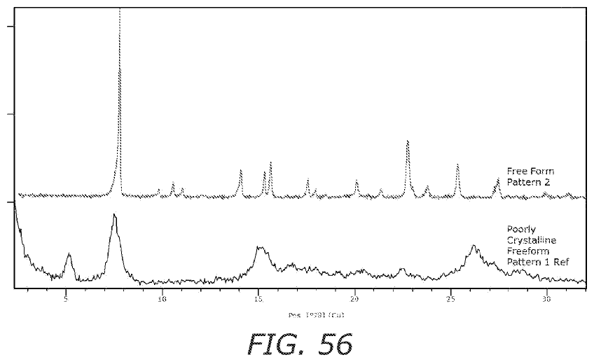

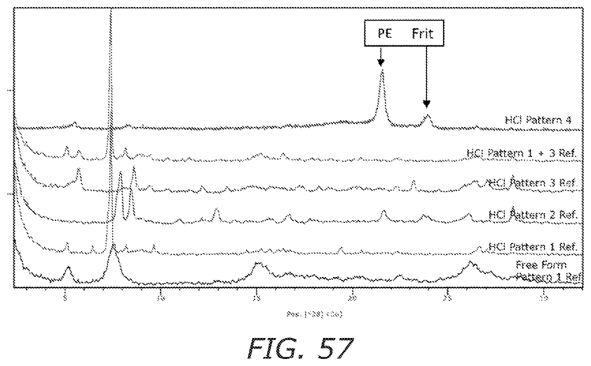

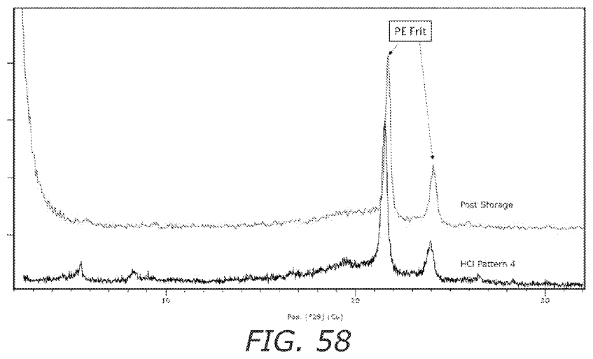

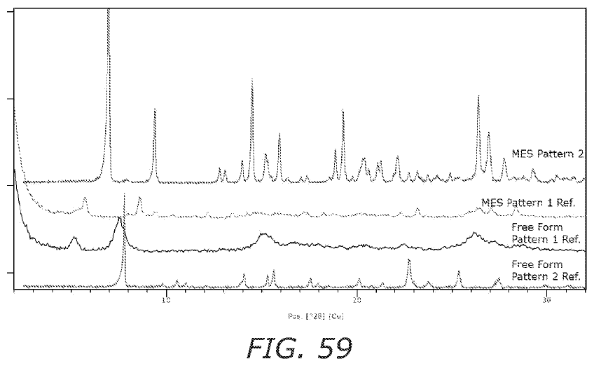

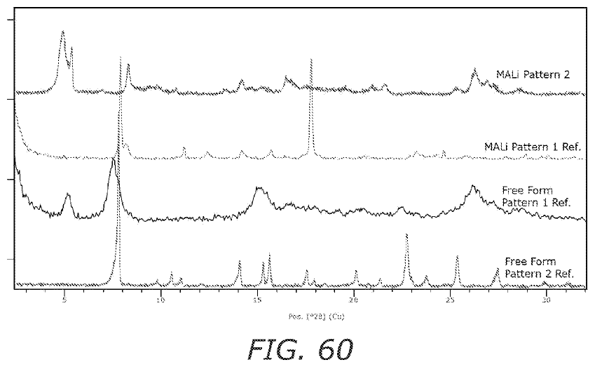

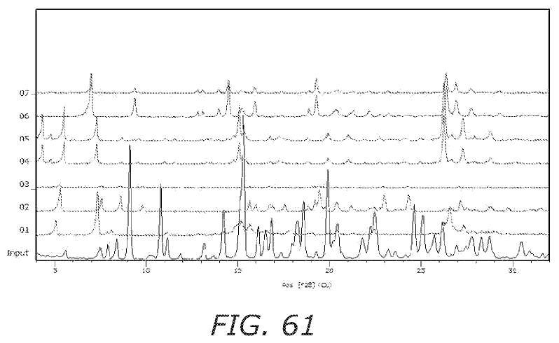

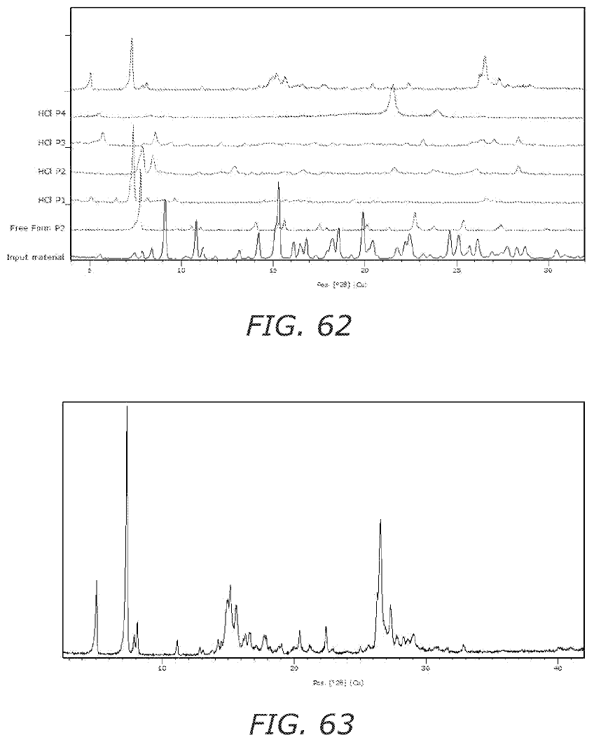

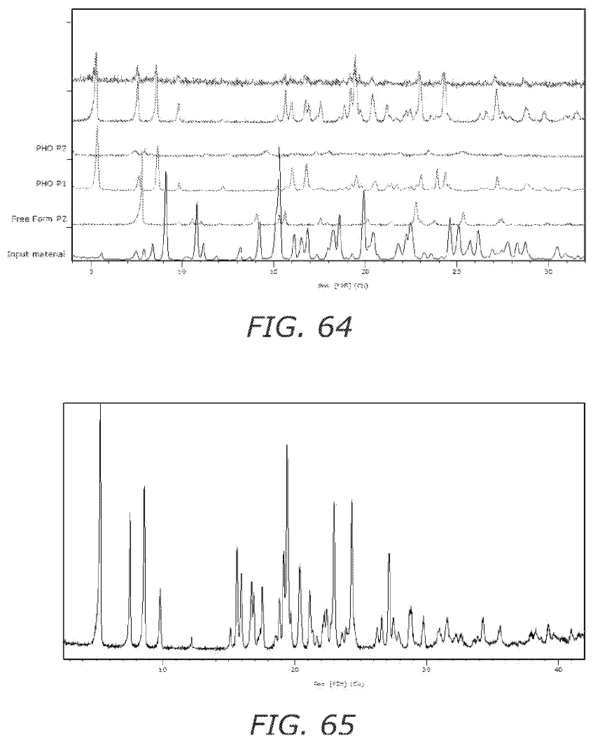

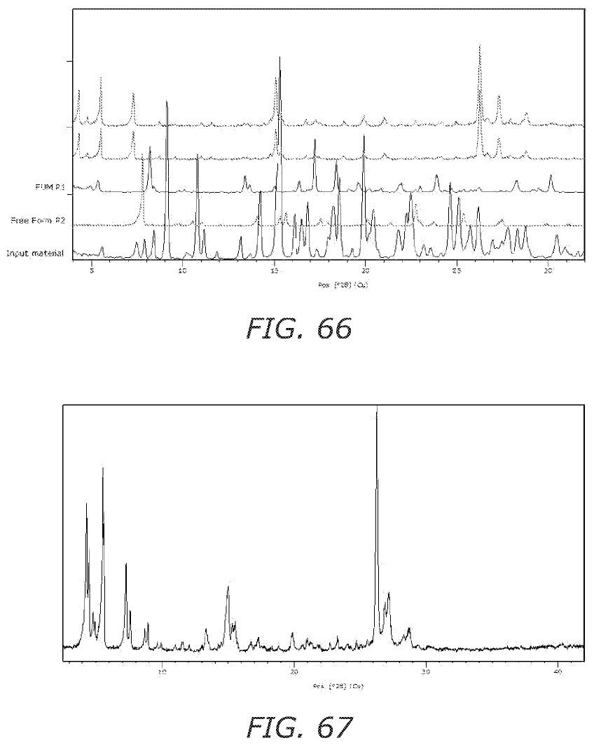

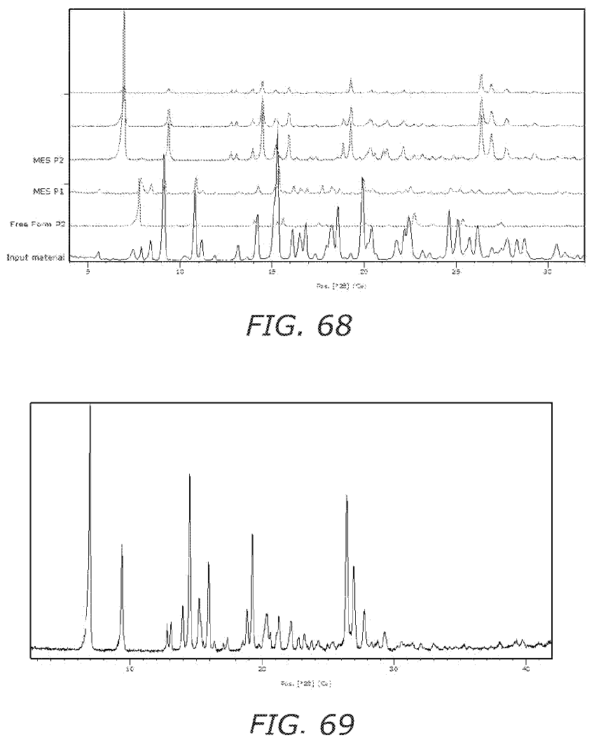

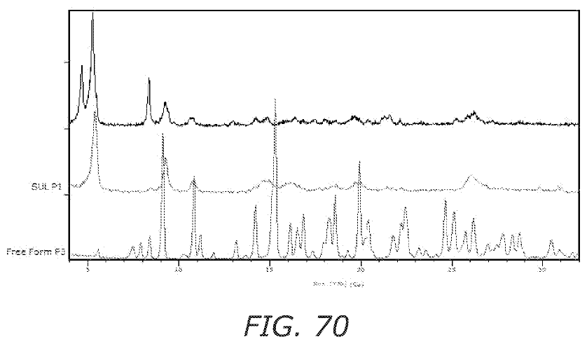

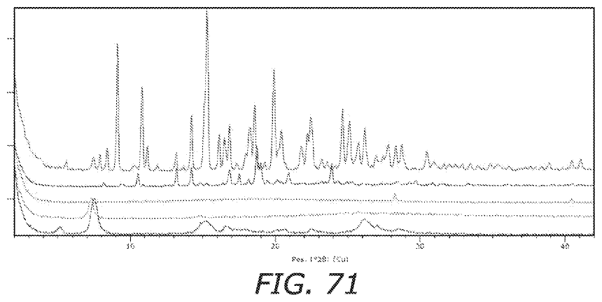

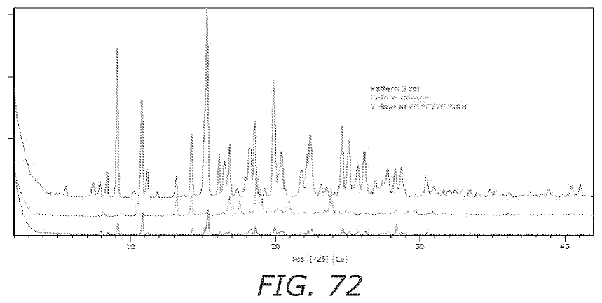

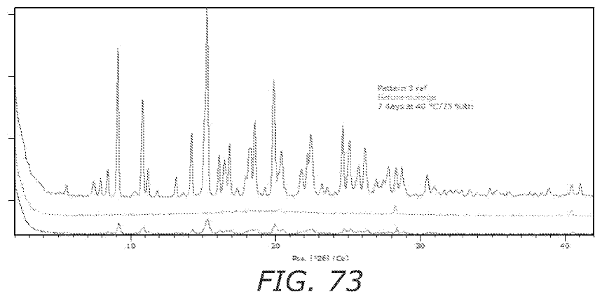

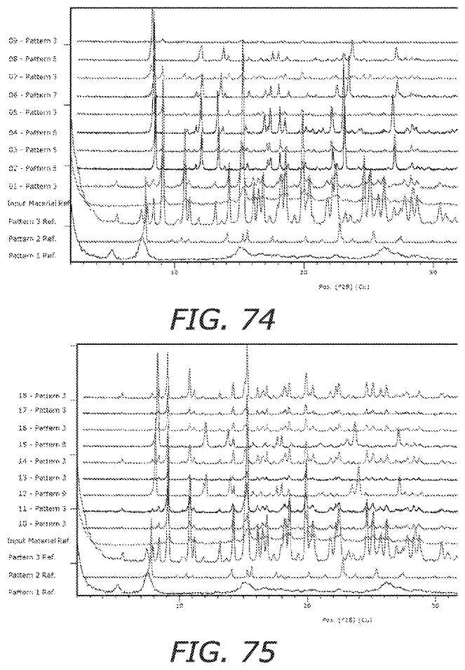

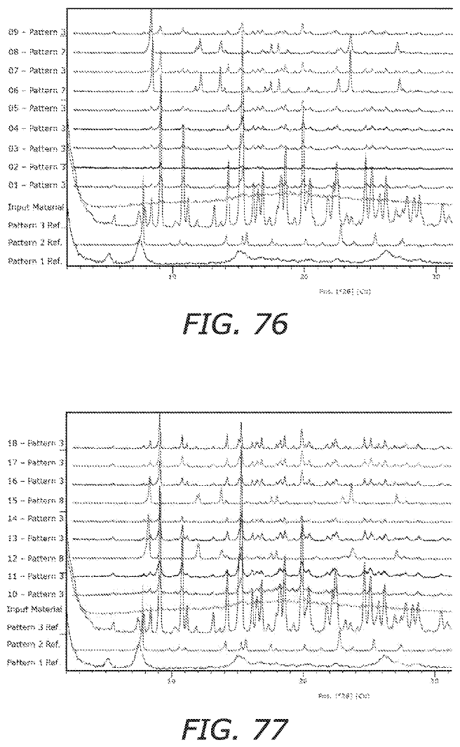

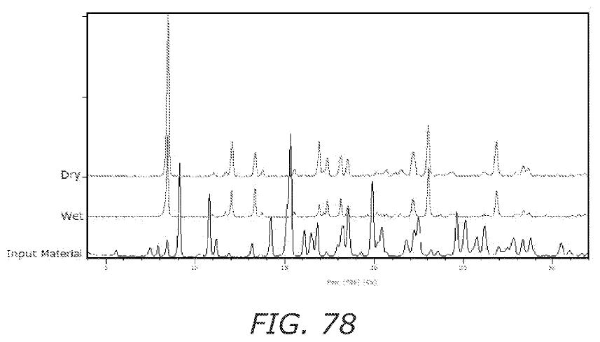

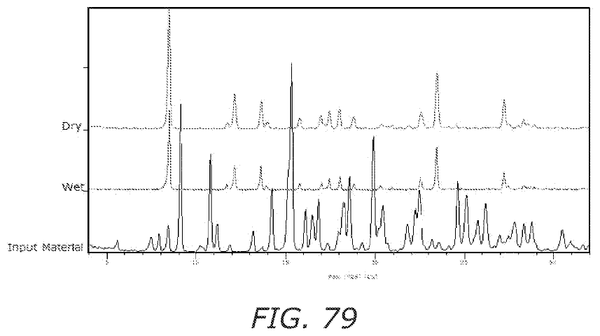

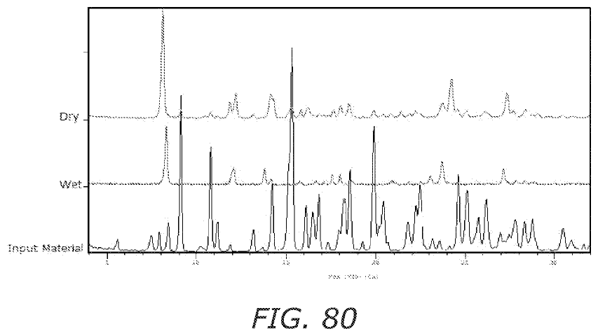

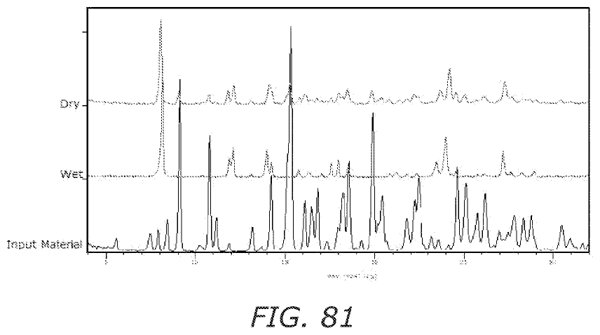

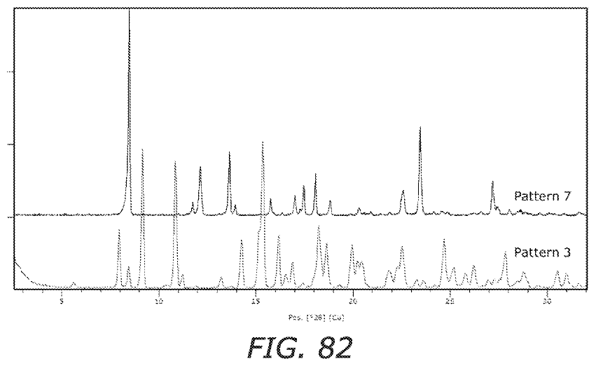

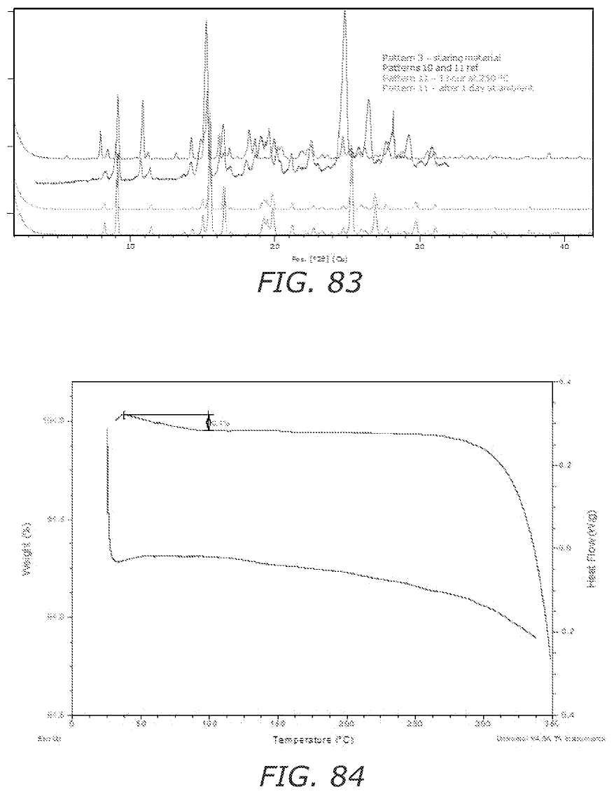

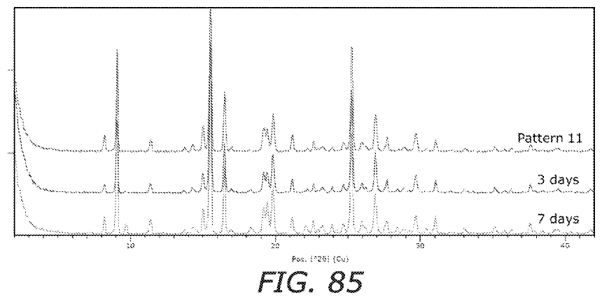

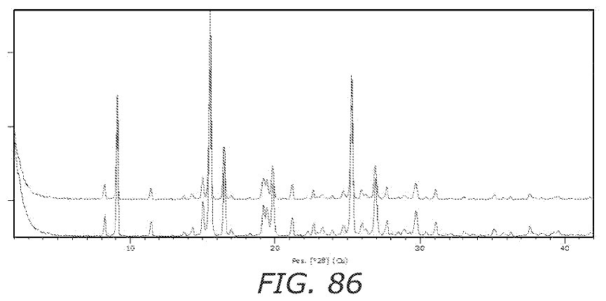

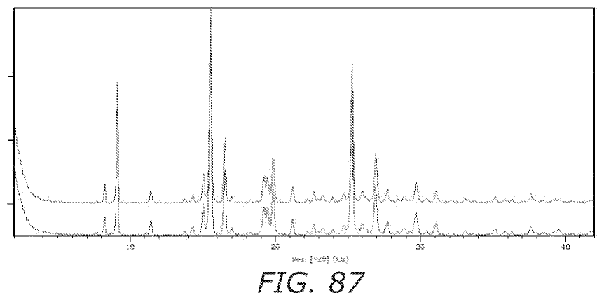

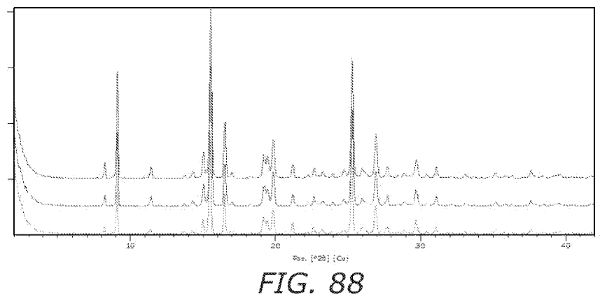

During the course of this investigation, 11 free form patterns of the compound of Structure (I) were identified. Pattern 1 was a poorly crystalline form. Pattern 2 was observed during the three experiments in the salt screen using material with a potassium impurity as input, high solvent content in one of the samples suggests it could be a THF solvate. Pattern 3 was a hemi-hydrate and was the prominent form obtained during both polymorph screens. Pattern 4 was obtained by reverse anti-solvent using dimethyl sulfoxide (DMSO) and tert-butyl methyl ether (TBME) and was determined to be a DMSO solvate. Patterns 5-9, isolated from the two polymorph screens, were a series of structurally related solvates. Pattern 10 was a dehydrated form of Pattern 3, only observed using in situ measurements during VT-XRPD and VAC-XRPD that converted back to Pattern 3 under ambient conditions. Pattern 11 was formed by heating Pattern 3 (via Pattern 10) at temperatures near 250° C. Pattern 11 was an anhydrous form and stable at room temperature. The relationships between the observed solid forms are summarized in FIG. 6 with each of the conditions used for the transitions as indicated:
1 Cooling with tetrahydrofuran (THF):water at a 9:1 ratio
2. Reverse anti-solvent DMSO/water
3. Reverse anti-solvent DMSO/TBME
4. 7 days at 40° C. at 75% relative humidity
5. Ambient conditions (following condition 4 above)
6. Under vacuum at 50° C. and heating at 175° C.
7 Heating at 250° C.
8. Slurry in fasted state simulated gastric fluid (FaSSGF) and pH 2.0 buffer
9. Dry grinding
10. 7 days at 40° C./70% relative humidity, slurry in various solvents at 50° C.
11. Slurry in ethyl acetate and isopropyl acetate at 50° C.
12. Slurry in methyl isobutyl ketone (MIBK) at 50° C.
13. Slurry in methanol at 50° C.
14. Slurry in ethanol and acetone at 50° C.
15. Slurry in methyl ethyl ketone (MEK) at 50° C.
16. Slurry in methanol and acetonitrile at 50° C.
17. Slurry in MEK and ethanol at 50° C.
18. Ambient conditions

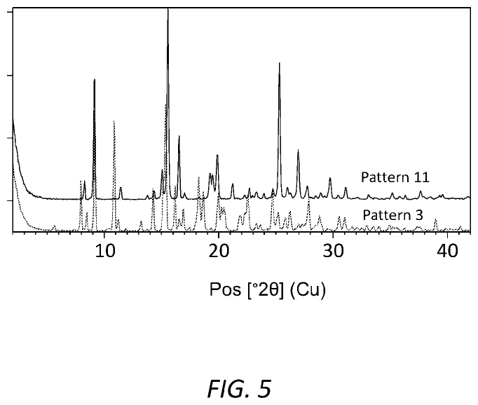

Of the identified forms of the compound of Structure (I), Pattern 3 and Pattern 11 have suitable solid state properties for use in a drug product. Pattern 3 is a hemi-hydrate with good stability to storage at elevated temperature and humidity conditions. Despite being hygroscopic (8.4 wt. % change 0-90% RH), the solid form was retained after the double-cycle GVS experiment. Pattern 11 is an anhydrous form and has good stability but, unlike Pattern 3, is only slightly hygroscopic (0.3 wt. % 0-90% RH). As Pattern 11 was formed via a high temperature form conversion, it would be necessary to investigate whether it can be obtained via a more scalable solution-based method. Measurement of the thermodynamic solubilities of both forms in simulated media and buffers at 25° C. did not reveal any significant advantage to one form over the other. A comparison of the XRPD patterns is shown in FIG. 5.

Competitive slurries were carried out using a mixture of Pattern 3 and Pattern 11 to determine the stability relationship between the two forms. The results of this were inconclusive, with some of the slurries remaining mixtures. The complex relationship between these forms may be a result of both temperature and water activity dependence. Additionally, this project aimed to identify salt forms of the compound of Structure (I) suitable solid state and physio-chemical properties for use in a drug product. It also investigated solid forms of the free form via a polymorph assessment with a view to identifying those forms with appropriate properties for the end use product.

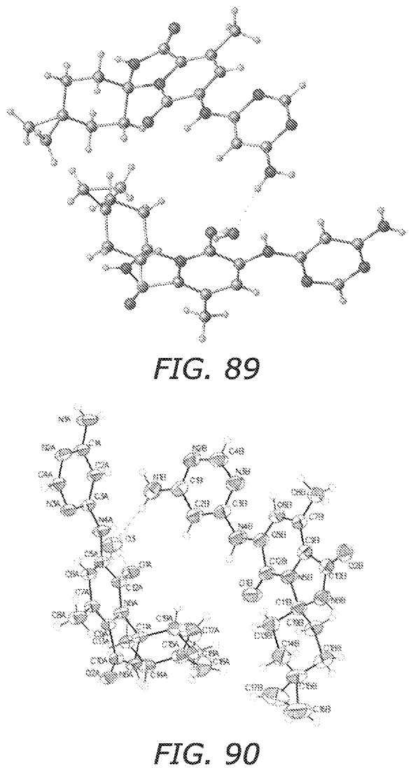

Two lots of material were used in these experiments. Lot A was determined to be Free Form Pattern 1 that was poorly crystalline, highly hygroscopic and had a 0.2 mol eq. potassium content. Lot B was characterized as Pattern 3 and was a hemi-hydrate, which although being hygroscopic, was found to be crystalline and stable. The single crystal structure for Pattern 3 was collected and is shown in FIGS. 89-92.

pKa measurements on the molecule showed it to have a basic pKa at 4.73 and acidic center at 11.49 and thus suitable could form salts at either end of the pH scale.

Experiments were performed to isolate salt forms of the compound of Structure (I). This has taken the form of a solubility and salt formation assessment and four salt screens. A standard screen with 12 common acids, a high temperature screen, a double equivalents screen and a targeted screen using high purity input material.

During this investigation, 12 salt forms were identified, along with two free base forms and some forms which were not unequivocally assigned. Some salts formed had properties that were not desirable for use as a drug product. Several of the most promising candidates (e.g PHO Pattern 1, MES Pattern 1) were characterized as hydrates, however, in each case the water was lost at temperatures close to ambient conditions, which may result in lower stability than desired.

A polymorphism assessment of the free form of Structure (I) was carried out. This consisted of two screens with 18 solvents, one carried out using poorly crystalline Pattern 3 as the input material and a second using amorphous compound of Structure (I) as input material.

During the course of the Examples described herein, 11 free form patterns of the compound of Structure (I) were identified. Pattern 1 was a poorly crystalline form that was first observed in the original material. Pattern 2 was only observed during the salt screen. Pattern 3 was a hemi-hydrate and was also the prominent form obtained during both polymorph screens. Pattern 4 was obtained by reverse anti-solvent using DMSO and TBME and was determined to be a DMSO solvate. Patterns 5-9, isolated from the two polymorph screens, were a series of structurally related solvates. Pattern 10 was a dehydrated form of Pattern 3, only observed using in situ measurements during VT-XRPD and VAC-XRPD, it converted back to Pattern 3 under ambient conditions. Pattern 11 was formed by heating Pattern 3 (via Pattern 10) to temperatures near 250° C. Pattern 11 was an anhydrous form and stable at room temperature.

Pattern 3 and Pattern 11 were free forms with suitable properties to scale-up and characterize further. Pattern 3 was hygroscopic although this does not affect the solid form, which was retained on return from high humidity levels to ambient conditions. Pattern 11 was only slightly hygroscopic. Pattern 11 was not accessed via a solution-based method, whereas Pattern 3 was a product of many of the screening experiments. Thermodynamic solubility data was collected for both forms in simulated fluids and buffers, the results were similar for both forms and so could not be used as a discriminating factor. Competitive slurry experiments were carried out in five solvents at three temperatures, these were ultimately inconclusive. It should be noted however, that none of the experiments during the cross-seeding experiments gave phase pure Pattern 3, all resulted in either a mixture of forms or Pattern 11.

### Example 20

### Preparation and Characterization of Lot a and Lot B of Structure (I)

Two lots of Structure (I) were used in the screening experiments described herein. They were prepared generally as described in WO 2023/278686. The final step to generate Structure (I) was performed as follows:

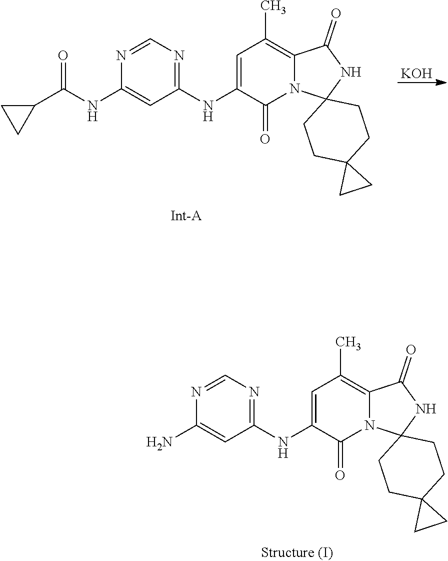

To a suspension of Int-A (1 equiv.) in EtOH/THF/H₂O (2:1:1, v/v/v, 25 vol.), an aqueous solution of potassium hydroxide (7.5 equiv. in 1 vol. of water) was added dropwise at 25-35° C. The reaction mixture was heated to 50-60° C. and stirred for 12-16 hours. The reaction mixture was then cooled to 25-35° C. Charcoal (20%) was added to the reaction mixture, and it was stirred for 1 hour. The reaction mixture was then filtered through a bed of Celite® and washed with a mixture of EtOH/THF/H₂O (2 vol.). SilametThiourea (20% w/w) was added to the filtrate and stirred for 1 hour. The suspension was filtered and the filtrate was passed through 0.2 micron filter paper, and the pH was adjusted to 7.5-8 using 1.5 N aq. HCl. The mixture was stirred for 1 hour at 0-5° C., and the precipitate was collected by filtration and washed with water. The isolated solid was slurried with purified water (10 vol.×2) for 30 minutes at 25-30° C. The solid was collected by filtration and washed with water (2 vol.). The solid was collected and dried at 45-50° C. for 3 hours to afford Structure (I).

### Characterization of Structure (I) Lot A

Lot A was prepared as described above in a batch size of 6.4 g.

Lot A was characterized using various techniques, as summarized in Table 39.

TABLE 39
Characterization of Lot A

| | |
|---|---|
| XRPD | Poorly crystalline Pattern 1 |
| ¹H-NMR | Consistent with structure, with all amine/amide protons |
| | observed. No residual solvent, but some impurities observed |
| | in baseline. |
| HPLC | 97.6% |
| IC | Cation: 0.2 mol eq. potassium |
| | Anion: 0.01 mol eq. sulfate |
| TGA | 7.8% weight loss between 25-160° C., followed by |
| | degradation beginning at approx. 260° C. |
| DSC | Broad endotherm with onset at 49.2° C. of 186 J/g, followed |
| | by an exotherm at 182.2° C. of 9 J/g |
| PLM | Crystalline plates and aggregates, largest of which are approx. |
| | 25 μm across |
| GVS and XRPD post- | Reversible mass change of 22.8-23.8% mass loss between 0- |
| GVS | 90% RH, exhibiting a slight hysteresis (ca. 6 mol eq water), |
| | highly hygroscopic. XRPD of the sample after the analysis |
| | showed that the solid form remained as poorly crystalline |
| | Pattern 1. |
| Storage at 40° C./75% | XRPD-poorly crystalline Pattern 1 (no change) |
| RH (8 days) | HPLC-98.2% |
| Storage at 25° C./97% | XRPD-poorly crystalline Pattern 1 (no change) |
| RH (8 days) | HPLC-97.5% |
| pKa | Basic: 4.73 |
| | Acidic: 11.49 |
| Thermodynamic | 0.13 mg/mL in SGF (pH 1.4) |
| solubility in simulated | 0.006 mg/mL in FaSSIF (pH 6.7) |
| media | 0.02 mg/mL in FeSSIF (pH 5.1) |

Lot A was determined to be poorly crystalline, with an XRPD pattern matching Pattern 1. It displayed a purity by HPLC of 97.6%, but IC showed that 0.2 mol eq. of potassium was present in the sample. Lot A was highly hygroscopic with a maximum of 23.8% mass change from 0-90% RH measured during a GVS experiment. The form by XRPD was unchanged during the experiment and was also unchanged when stored under elevated temperature and/or humidity conditions for 8 days. Structure (I) Pattern 1 is slightly soluble in SGF (0.13 mg/mL), and practically insoluble in FaSSIF (0.006 mg/mL) and FeSSIF (0.02 mg/mL) according to USP guidance.

### Characterization of Structure (I) Lot B

Lot B was prepared as described above in a batch size of 11.8 g, except that the pH was adjusted to 8-9 using 1.5 N aq. HCl.

Lot B was characterized using various techniques, as summarized in Table 40.

TABLE 40
Characterization of Lot B

| | Sample 1 | Sample 2 |
|---|---|---|
| XRPD | Pattern 3 | Pattern 3, missing peaks at |
| | | 7.5 and 40.5° |
| ¹H-NMR | Consistent with structure; no residual | ND |
| | solvent. | |
| HPLC | 98.5% | 98.1% |
| IC | 0.04 mol eq. chloride | 0.01 eq. chloride, no cations |
| | 0.09 mol eq. potassium | detected |
| TGA | 1.0 wt % loss RT-90° C. (0.21 mol eq. | ND |
| | water) | |
| | 2.0 wt % loss 90-160° C. (0.42 mol eq. | |
| | water) | |
| | 0.6 wt % loss 160-265° C. (0.12 mol | |
| | eq. water) | |
| DSC | Broad endotherm onset 89.5° C. (68 | ND |
| | J/g) | |
| | Exotherm onset 213.5° C. (37 J/g) | |
| KF | 3.4 wt % water (0.72 mol eq. water) | ND |
| PLM | Soft agglomerates of plate shaped | ND |
| | particles up to 70 μm | |
| GVS and | 8.4 wt % reversible water uptake | ND |
| XRPD post- | 0-90% RH | |
| GVS | 6.3 wt % uptake 80-90% RH | |
| | Some hysteresis 40-90% RH | |
| | Post GVS XRPD: No change in form | |
| | Material is hygroscopic | |
| Storage at | Unchanged by XRPD, 98.8% purity | ND |
| 40° C./75% RH | | |
| (7 days) | | |
| Storage at | Unchanged by XRPD, 98.5% purity | ND |
| 25° C./97% RH | | |
| (8 days) | | |
| XRPD under | Conversion to Pattern 10 after 30 min | ND |
| vacuum | under vacuum | |
| | Conversion back to Pattern 3 after | |
| | vacuum released for 30 min | |
| VT-XRPD | 25-80° C.: Pattern 3 | ND |
| | 175° C.: Pattern 10 | |
| | 250° C.: Patterns 10 and 11 | |
| | Cooling to 25° C.: Patterns 11 and 3 | |
| Solubility | ND | FaSSGF-0.026 mg/mL |
| (25° C./24 h) | | FeSSIF-0.0060 mg/mL |
| | | FaSSIF-0.0016 mg/mL |
| | | pH 2.0-0.012 mg/mL |
| | | pH 7.4-0.001 mg/mL |
| LogD | ND | 3.10 |

ND = not determined

Two samples from Lot B were determined to be Pattern 3. IC analysis showed less ions present in these batches than in Lot A, in particular Sample 2 of Lot B only had 0.01 mol eq. of chloride present. The solid form and chemical purity remained unchanged when the sample was stored at elevated temperature and humidity.

Water content analysis by KF showed the material to contain 3.4 wt. % of water (0.72 mol eq. of water). This is consistent with the TGA data, which showed a total mass loss of 3.6 wt. % from ambient to 265° C. On reduction of the humidity to 0% during the GVS experiment, a mass loss of 0.8 wt % was observed, suggesting that at least some of the water (remaining 2.6% based on KF result) is tightly bound and likely part of the crystal structure rather than surface bound. Based on these data, the material was assigned to be a hemi-hydrate.

The nature of the hydrate was investigated further by vacuum XRPD and variable temperature XRPD. When put under vacuum Lot B (Pattern 3) was converted to Pattern 10, presumably a dehydrated form of this material. When Pattern 10 was exposed to ambient conditions for 30 minutes it converted back to Pattern 3. When Pattern 3 was heated to 175° C., Pattern 10 was again observed; this was consistent with the temperature at which 3.0% of the mass loss observed in the TGA has occurred. On heating this material further to 250° C., Pattern 10 partially converted to Pattern 11. Pattern 10 in this physical mixture converted to Pattern 3 on returning to ambient conditions, whilst the Pattern 11 remained unchanged. These experiments suggest Pattern 3 is a hemi-hydrate which can be dehydrated to give Pattern 10, which will in turn readily re-hydrate under ambient conditions. Pattern 11 is formed by heating Pattern 10 to near 250° C., seen as an exotherm in the DSC, and appears to be stable under ambient conditions.

The solubility of Structure (I) Pattern 3 was determined to be poor in simulated fluids and buffers, with the highest solubility being observed in FaSSGF (0.026 mg/mL).

### Example 21

### Single Crystal X-Ray Diffraction Analysis of Free Form Pattern 3

Crystals of Structure (I) Pattern 3 were obtained by evaporation of a THF:water 9:1 solution. A crystal of Structure (I) Pattern 3 of sufficient size and quality for analysis by single crystal X-ray diffraction was isolated from the sample with approximate dimensions 0.15×0.05×0.02 mm.

The single crystal X-ray structure of Structure (I) Pattern 3 was determined at 293(2) K and a summary of the structural data can be found in Table 41. The crystal structure of Structure (I) Pattern 3 was solved in the triclinic space group P-1 with the final R1 [I>2σ(I)]=4.36%. The structure was identified as depicted in FIG. 89 and FIG. 90 and the asymmetric unit found to contain two molecules of Structure (I).

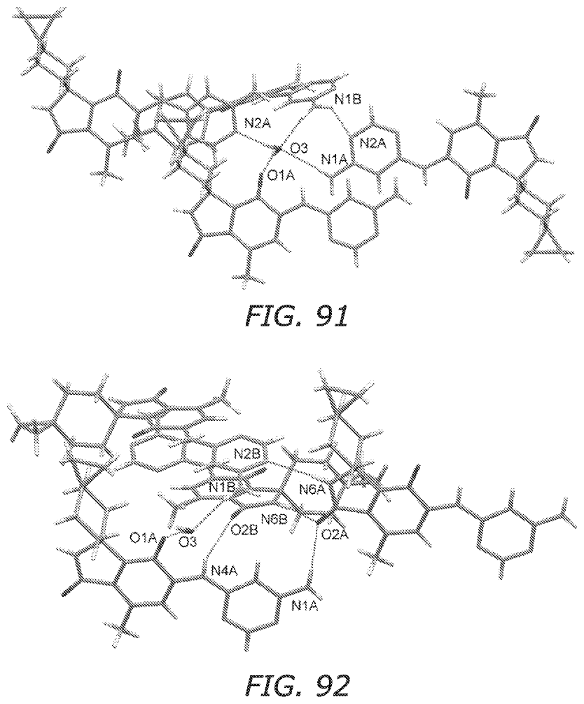

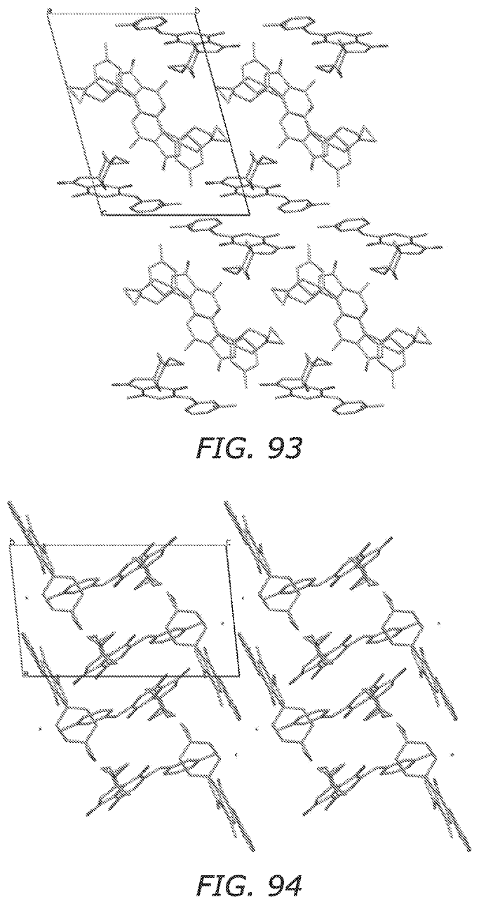

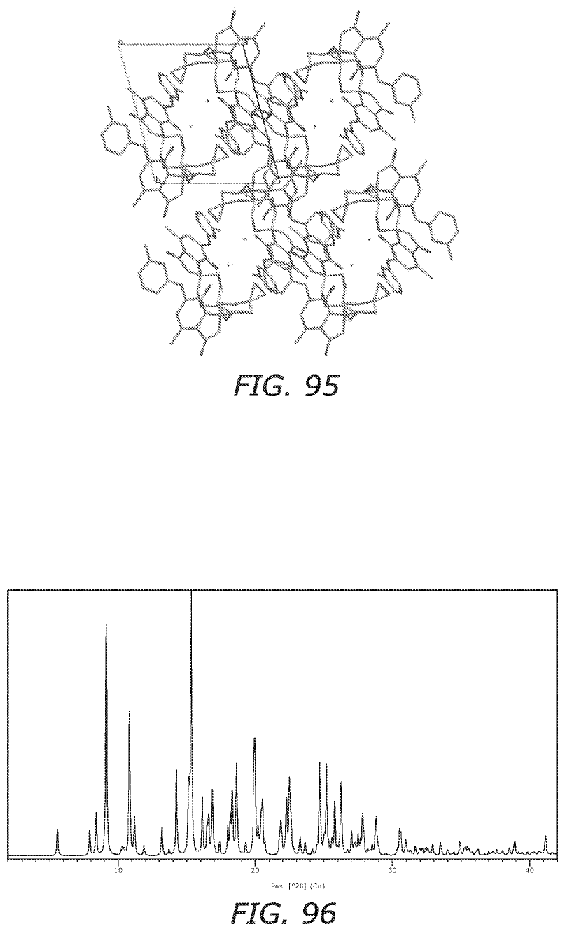

FIG. 91 and FIG. 92 show the hydrogen bonding network of Structure (I) Pattern 3 with the intermolecular hydrogen bonds shown as dashed lines. FIGS. 93-95 show views of part of the crystal packing in the unit cell looking down the crystallographic a-, b-, and c-axes respectively. For clarity all hydrogen atoms have been removed from packing diagrams.

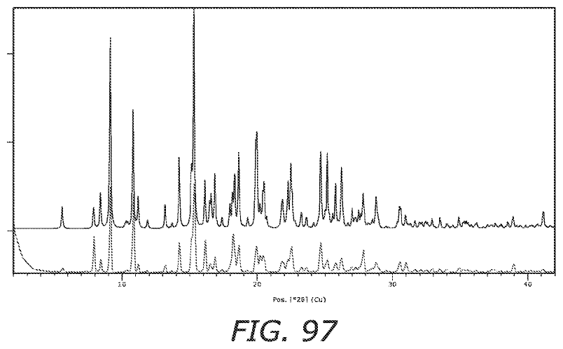

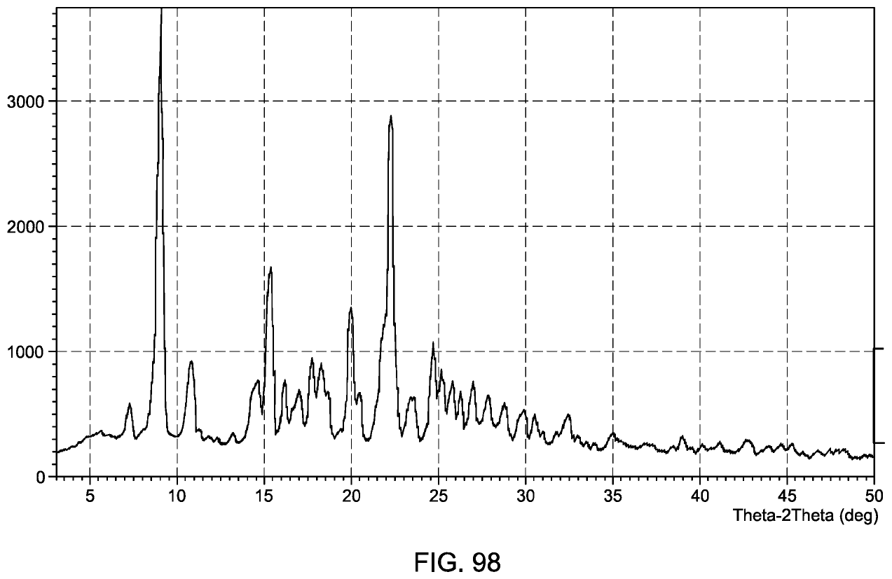

The simulated XRPD pattern of Structure (I) Pattern 3 at (293(2) K) is shown in FIG. 96. The overlay in FIG. 97 shows a comparison between an experimental diffractogram collected at RT and the pattern simulated from the single crystal data at 293 K. The patterns were consistent, which confirmed that the single crystal used for the structure determination was representative of the reference material. Slight differences in the simulated and experimental diffractograms were attributable to preferred orientation.

TABLE 41
Sample and crystal data for Structure (I) Pattern 3

| Crystallization solvents | THF:Water 9:1 | |
|---|---|---|
| Crystallization method | Evaporative | |
| Empirical formula | C₁₉H₂₃N₆O_{2.5} | |
| Formula weight | 375.43 | |
| Temperature | 293(2) | K |
| Wavelength | 1.54184 | Å |
| Crystal size | 0.150 × 0.050 × 0.020 mm | |
| Crystal habit | Colorless lath | |
| Crystal system | Triclinic | |
| Space group | P-1 | |
| Unit cell dimensions | a = 10.1823(3) Å | α = 72.512(2)° |
| | b = 12.1136(4) Å | β = 79.465(2)° |
| | c = 16.6675(3) Å | γ = 72.661(3)° |
| Volume | 1861.87(10) | Å³ |
| Z | 4 | |
| Density (calculated) | 1.339 | Mg/m³ |
| Absorption coefficient | 0.756 | mm⁻¹ |

| | |
|---|---|
| F(000) | 796 |

### Example 22

### Water Slurry Experiments of Free Form Pattern 3

In certain batches of Structure (I) Pattern 3, additional peaks were observed in the XRPD spectrum (see, e.g., FIG. 98) and a water content greater than that expected for a hemihydrate was measured. For purposes of consistency, studies were conducted to determine conditions that would convert a mixture of forms into Structure (I) Pattern 3 only.

About 10 g of material comprising Structure (I) Pattern 3 with additional peaks and increased water content (e.g., as shown in FIG. 98) was tested under reprocessing conditions to isolate an initial solid after pH adjustment, as follows. About 10 g aliquot of the material was dissolved in a mixture of EtOH/THF/H₂O (2:1:1/V:V:V, 25 V) and KOH (7.0 eq). The reaction mass was stirred for 10 min to give a clear solution. Then, the pH of the reaction was adjusted to 7.5-8 using 1.5 N aq. HCl. The precipitated solids were stirred for 1 hour at 0-5° C., and then the solids were collected by filtration and washed with purified water.

The isolated solids were then divided into two parts (Part-1 and Part-2). Part-1 material was dried at 50° C. for 48 h in VTD under reduced pressure. Part-2 material was subjected to a water slurry at 25-30° C. for 1 h, followed by drying at 50° C. for 48 h in VTD under reduced pressure. Samples from Part-1 and Part-2 were analyzed with XPRD and for water content. Part-1 material partially conformed with Pattern-3 and had a water content of 3.1% w/w, while Part-2 material was similar to the material before reprocessing and had a water content of 4.8% w/w.

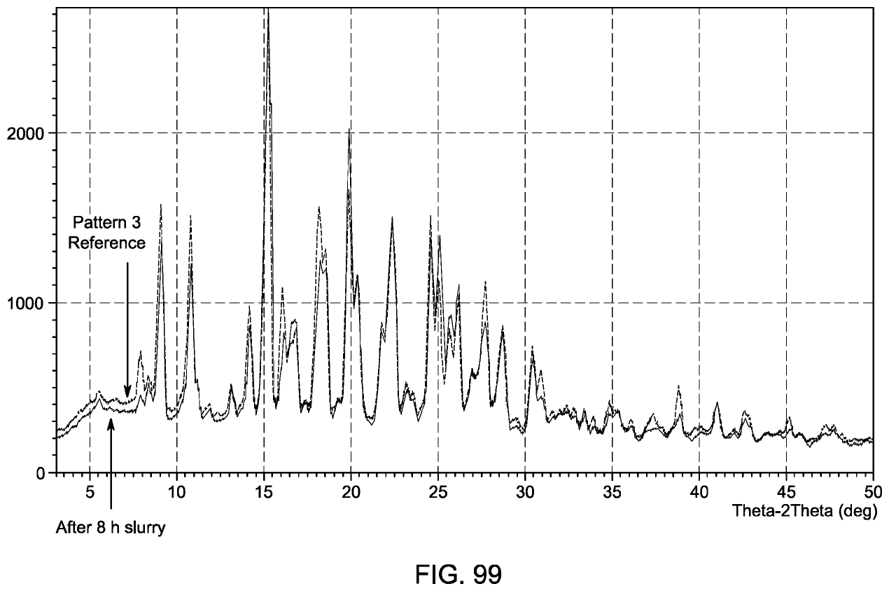

About 2 g of the Part-2 material was subjected to further water slurry, using a stir bar, for 8 h at 25-30° C., followed drying in VTD under reduced pressure at 50° C. for 48 h. The obtained material was analyzed with XRPD and for water content and was found to conform with Pattern 3 (FIG. 99) and having a water content of 2.3% w/w.

To confirm reproducibility of these results, an additional experiment was performed with the same material comprising Structure (I) Pattern 3 with additional peaks and increased water content. The material was not subjected to reprocessing as described above but was slurried in water for 8 h at 25-30° C. One aliquot (5 g) was slurried in water with a stirring bar, and another aliquot (5 g) was slurried in water with an overhead stirrer. After slurrying, both aliquots were dried in VTD at 50° C. for 48 h and then analyzed for XRPD and water content. The material obtained by slurrying with a stirring bar conformed with Pattern 3 and had a water content of 2.8% w/w. The material obtained by slurrying with an overhead stirrer partially conformed with Pattern 3 and had a water content of 3.6% w/w. Resubjecting this material to an additional 16 hours of slurrying in water at 25-30° C. with an overhead stirrer resulted in material that fully conformed with Pattern 3 and had a water content of 2.6% w/w.

### Example 23

### Characterization Studies of Free Form Pattern 3

Batches of Structure (I) Pattern 3 with additional peaks, e.g., as described in Example 22, were characterized further.

Material comprising Structure (I) Pattern 3 with additional peaks (e.g., such as in FIG. 98) was micronized using an air jet miller targeting D90<20 microns. The following conditions were used: primary nitrogen pressure of 6 kg/cm²; secondary nitrogen pressure of 6 kg/cm²; nitrogen atmosphere; room temperature; 75 g input quantity; and 62 g output quantity after micronization. The micronized material was analyzed using XRPD. Only slight changes in the XRPD spectrum were observed, and the additional peaks were still present. After micronization, the particle size target of D90<20 microns was achieved. Attempted drying of a sample after micronization at 50° C. for 24 h did not result in a change in water content.

The input material from Example 22 and the jet-milled batch were analyzed using variable temperature XRPD:

| Temperature (° C.) | Example 22 material | Jet-milled batch |
|---|---|---|
| 25 | Pattern 3 with additional peaks | Pattern 3 with additional peaks |
| 75 | Pattern 10 with additional peaks | Pattern 3 with additional peaks, |
| | | peak shifting observed |
| 100 | Pattern 10 with additional peaks | Unchanged |
| 125 | Pattern 10 with additional peaks | Unchanged |
| 150 | Pattern 10 with additional peaks | Pattern 10 with additional peaks |
| 175 | Pattern 10 with additional peaks | Pattern 10 with additional peaks |
| 200 | Pattern 10 with additional peaks | Pattern 10 and Pattern 11 |
| | and missing peaks | |
| 250 | Pattern 11 | Pattern 11 with additional peaks |
| Cooling to 25 | Pattern 11 | Pattern 11 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A solid form of a compound having the following Structure (I): 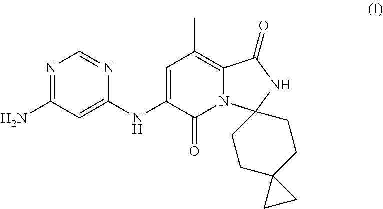 [SMILES: Cc1cc(Nc2cc(N)ncn2)c(=O)n2c1C(=O)NC21CCC2(CC2)CC1]
or a tautomer thereof, wherein the solid form has an X-ray powder diffraction pattern with at least two peaks at 2-theta angles selected from the group consisting of 5.6±0.2°, 10.9±0.2°, 18.2±0.2°, and 18.6±0.2°.

2. The solid form of claim 1, wherein the solid form has an X-ray powder diffraction pattern with at least three peaks at 2-theta angles selected from the group consisting of 5.6±0.2°, 10.9±0.2°, 18.2±0.2°, and 18.6±0.2°.

3. The solid form of claim 1, wherein the solid form has an X-ray powder diffraction pattern with peaks at 2-theta angles at 5.6±0.2°, 10.9±0.2°, 18.2±0.2°, and 18.6±0.2°.

4. The solid form of claim 1, wherein the solid form has an X-ray powder diffraction pattern with peaks at 2-theta angles at 5.6±0.2°, 8.0±0.2°, 8.4±0.2°, 9.2±0.2°, 10.9±0.2°, 11.2±0.2°, 13.2±0.2°, 14.3±0.2°, 15.3±0.2°, 16.2±0.2°, 16.5±0.2°, 16.9±0.2°, 17.4±0.2°, 18.2±0.2°, 18.6±0.2°, 19.9±0.2°, 20.2±0.2°, 20.5±0.2°, 21.9±0.2°, 22.3±0.2°, 22.5±0.2°, 23.3±0.2°, 23.6±0.2°, 24.7±0.2°, 25.2±0.2°, 25.8±0.2°, 26.2±0.2°, 27.0±0.2°, 27.3±0.2°, 27.8±0.2°, 28.5±0.2°, and 28.8±0.2°.

5. A solid form of a compound having the following Structure (I): 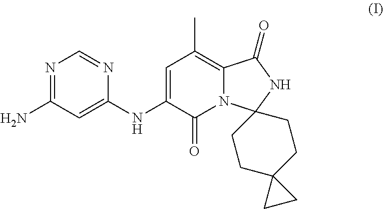 [SMILES: Cc1cc(Nc2cc(N)ncn2)c(=O)n2c1C(=O)NC21CCC2(CC2)CC1]
or a tautomer thereof, having an X-ray powder diffraction pattern substantially in accordance with that depicted in FIG. 1.

6. The solid form of any one of claims 1-5, characterized by a differential scanning calorimetry thermogram comprising an endothermic peak with an onset of about 89.5° C.

7. The solid form of any one of claims 1-6, characterized by a differential scanning calorimetry thermogram comprising an exothermic peak with an onset of about 213.5° C.

8. The solid form of any one of claims 1-7, characterized by a differential scanning calorimetry thermogram substantially in accordance with that depicted in FIG. 2.

9. A solid form of a compound having the following Structure (I): 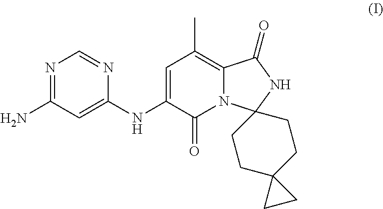 [SMILES: Cc1cc(Nc2cc(N)ncn2)c(=O)n2c1C(=O)NC21CCC2(CC2)CC1]
or a tautomer thereof, wherein the solid form is prepared by a process comprising steps of:
(i) providing N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide (Int-A);
(ii) contacting Int-A with a hydroxide base (e.g., potassium hydroxide) in a suitable solvent (e.g., ethanol, tetrahydrofuran, and water, or a mixture thereof); and
(iii) isolating the solid form of a compound having Structure (I).

10. The solid form of claim 9, further comprising a step of slurrying the solid form of a compound having Structure (I) in a suitable solvent (e.g., water).

11. The solid form of any one of claims 1-10, wherein the solid form is a hemihydrate.

12. A solid form of a compound having the following Structure (I): 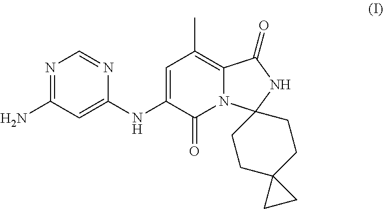 [SMILES: Cc1cc(Nc2cc(N)ncn2)c(=O)n2c1C(=O)NC21CCC2(CC2)CC1]
or a tautomer thereof, wherein the solid form has an X-ray powder diffraction pattern with at least two peaks at 2-theta angles selected from the group consisting of 19.2±0.2°, 19.5±0.2°, and 21.2±0.2°.

13. The solid form of claim 12, wherein the solid form has an X-ray powder diffraction pattern with peaks at 2-theta angles at 19.2±0.2°, 19.5±0.2°, and 21.2±0.2°.

14. The solid form of claim 12, wherein the solid form has an X-ray powder diffraction pattern with peaks at 2-theta angles at 8.2±0.2°, 9.1±0.2°, 11.4±0.2°, 13.8±0.2°, 14.3±0.2°, 15.0±0.2°, 15.5±0.2°, 16.5±0.2°, 17.0±0.2°, 19.2±0.2°, 19.5±0.2°, 19.9±0.2°, 21.2±0.2°, 22.3±0.2°, 22.7±0.2°, 23.3±0.2°, 23.9±0.2°, 24.7±0.2°, 25.3±0.2°, 26.0±0.2°, 26.9±0.2°, 27.7±0.2°, 28.5±0.2°, 28.9±0.2°, and 29.7±0.2°.

15. A solid form of a compound having the following Structure (I): 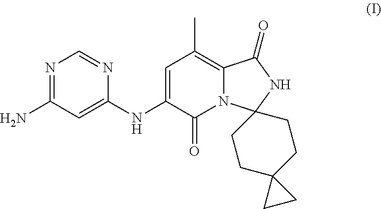 [SMILES: Cc1cc(Nc2cc(N)ncn2)c(=O)n2c1C(=O)NC21CCC2(CC2)CC1]
or a tautomer thereof, having an X-ray powder diffraction pattern substantially in accordance with that depicted in FIG. 3.

16. The solid form of any one of claims 12-15, characterized by a differential scanning calorimetry thermogram comprising no events up to about 340° C.

17. The solid form of any one of claims 12-15, characterized by a differential scanning calorimetry thermogram substantially in accordance with that depicted in FIG. 4.

18. A solid form of a compound having the following Structure (I): 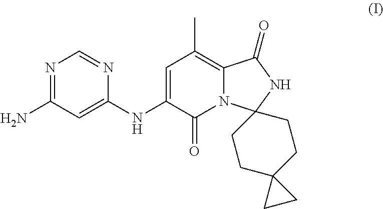 [SMILES: Cc1cc(Nc2cc(N)ncn2)c(=O)n2c1C(=O)NC21CCC2(CC2)CC1]
or a tautomer thereof, wherein the solid form is prepared by a process comprising steps of:
(i) providing Structure (I) Pattern 3;
(ii) heating Structure (I) Pattern 3 to about 250° C. under vacuum; and
(iii) isolating the solid form of a compound having Structure (I).

19. The solid form of any one of claims 12-18, wherein the solid form is unsolvated.

20. An amorphous solid form of a compound having the following Structure (I): 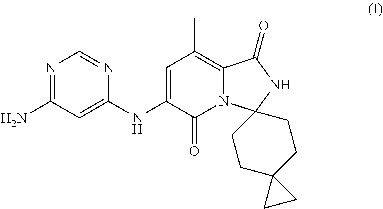 [SMILES: Cc1cc(Nc2cc(N)ncn2)c(=O)n2c1C(=O)NC21CCC2(CC2)CC1]
or a tautomer thereof.

21. An amorphous solid form of a compound having the following Structure (I): 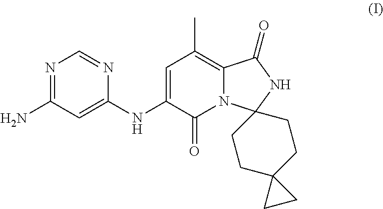 [SMILES: Cc1cc(Nc2cc(N)ncn2)c(=O)n2c1C(=O)NC21CCC2(CC2)CC1]
or a tautomer thereof, wherein the amorphous form is prepared by a process comprising steps of:
(i) providing Structure (I) Pattern 3;
(ii) ball milling Structure (I) Pattern 3 at a suitable frequency (e.g., 30 Hz) and for a suitable amount of time (e.g., 3 sessions of 90 min each); and
(iii) isolating the amorphous form of a compound having Structure (I).

22. A crystalline solid form of a compound having the following Structure (I): 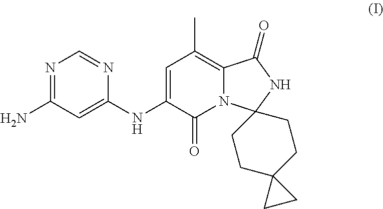 [SMILES: Cc1cc(Nc2cc(N)ncn2)c(=O)n2c1C(=O)NC21CCC2(CC2)CC1]
or a tautomer thereof, wherein the crystalline solid form is described in the Example herein.

23. A crystalline solid form of a compound having the following Structure (I): 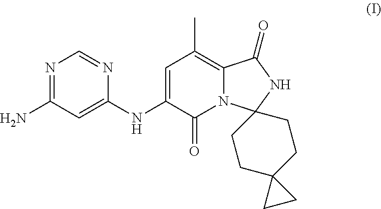 [SMILES: Cc1cc(Nc2cc(N)ncn2)c(=O)n2c1C(=O)NC21CCC2(CC2)CC1]
or a tautomer thereof, wherein the crystalline solid form is prepared by a process comprising steps of:
(i) providing Structure (I);
(ii) contacting Structure (I) with one or more suitable solvents (e.g., n-heptane, ethyl acetate, isopropyl acetate, methyl isobutyl ketone, 2-propanol, methyl ethyl ketone, acetone, ethanol, tert-butyl methyl ether, 2-methyl-1-propanol, cyclohexane, methanol, toluene, tetrahydrofuran, acetonitrile, water, dimethylsulfoxide, or a combination thereof); and
(iii) isolating the crystalline solid form,
the process optionally comprising a step of heating or cooling the mixture of Structure (I) in a suitable solvent or a solid precipitate isolated therefrom.

24. A salt form of a compound having the following Structure (I): 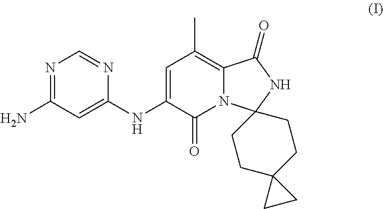 [SMILES: Cc1cc(Nc2cc(N)ncn2)c(=O)n2c1C(=O)NC21CCC2(CC2)CC1]
or a tautomer thereof, wherein the salt form is formed between Structure (I) and a co-former selected from hydrochloric acid, hydrobromic acid, sulfuric acid, acetic acid, maleic acid, fumaric acid, phosphoric acid, citric acid, p-toluenesulfonic acid, methanesulfonic acid, benzenesulfonic acid, tartaric acid, succinic acid, and malic acid.

25. The salt form of claim 24, wherein the salt form is described in the Examples herein.

26. The salt form of claim 24 or 25, wherein the salt form is crystalline.

27. A salt form of a compound having the following Structure (I): 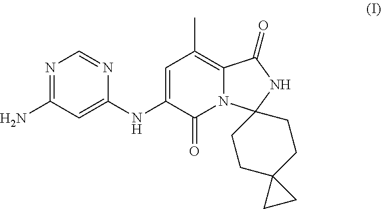 [SMILES: Cc1cc(Nc2cc(N)ncn2)c(=O)n2c1C(=O)NC21CCC2(CC2)CC1]
or a tautomer thereof, wherein the salt form is prepared by a process comprising steps of:
(i) providing Structure (I);
(ii) contacting Structure (I) with a co-former selected from hydrochloric acid, hydrobromic acid, sulfuric acid, acetic acid, maleic acid, fumaric acid, phosphoric acid, citric acid, p-toluenesulfonic acid, methanesulfonic acid, benzenesulfonic acid, tartaric acid, succinic acid, and malic acid; and
(iii) isolating the salt form.

28. A solid form (e.g., a crystalline or amorphous form of a free base or salt form) of a compound having the following Structure (I): 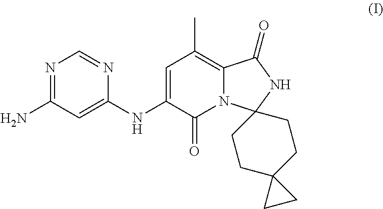 [SMILES: Cc1cc(Nc2cc(N)ncn2)c(=O)n2c1C(=O)NC21CCC2(CC2)CC1]
or a tautomer thereof, wherein the solid form is prepared by a process described in the Examples herein.

29. A pharmaceutical composition comprising a solid form of any one of claims 1-28 and a pharmaceutically acceptable carrier or excipient.

30. The pharmaceutical composition of claim 29, formulated for oral administration.

31. The pharmaceutical composition of claim 29, in the form of a capsule.

32. The pharmaceutical composition of claim 29, in the form of a tablet.

33. A method of preparing a solid form of a compound having the following Structure (I): 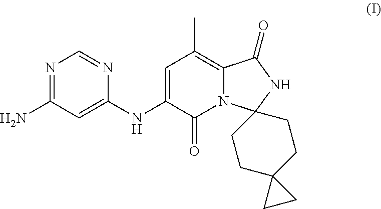 [SMILES: Cc1cc(Nc2cc(N)ncn2)c(=O)n2c1C(=O)NC21CCC2(CC2)CC1]
or a tautomer thereof, the method comprising steps of:
(i) providing N-(6-((8″-methyl-1″,5″-dioxo-1″,5″-dihydro-2″H-dispiro[cyclopropane-1,1′-cyclohexane-4′,3″-imidazo[1,5-a]pyridin]-6″-yl)amino)pyrimidin-4-yl)cyclopropanecarboxamide (Int-A);
(ii) contacting Int-A with a hydroxide base (e.g., potassium hydroxide) in a suitable solvent (e.g., ethanol, tetrahydrofuran, and water, or a mixture thereof); and
(iii) isolating the solid form of a compound having Structure (I).

34. The method of claim 33, further comprising a step of slurrying the solid form of a compound having Structure (I) in a suitable solvent (e.g., water).

35. A method of preparing a solid form of a compound having the following Structure (I): 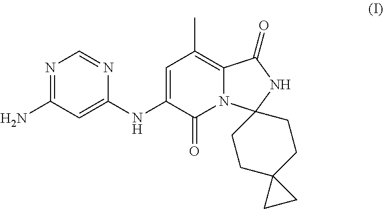 [SMILES: Cc1cc(Nc2cc(N)ncn2)c(=O)n2c1C(=O)NC21CCC2(CC2)CC1]
or a tautomer thereof, the method comprising steps of:
(i) providing Structure (I) Pattern 3;
(ii) heating Structure (I) Pattern 3 to about 250° C. under vacuum; and
(iii) isolating the solid form of a compound having Structure (I).

36. A method of preparing an amorphous form of a compound having the following Structure (I): 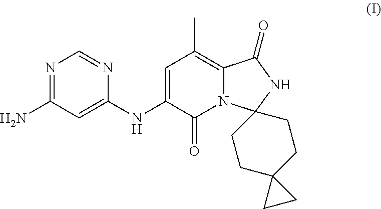 [SMILES: Cc1cc(Nc2cc(N)ncn2)c(=O)n2c1C(=O)NC21CCC2(CC2)CC1]
or a tautomer thereof, the method comprising steps of:
(i) providing Structure (I) Pattern 3;
(ii) ball milling Structure (I) Pattern 3 at a suitable frequency (e.g., 30 Hz) and for a suitable amount of time (e.g., 3 sessions of 90 min each); and
(iii) isolating the amorphous form of a compound having Structure (I).

37. A method of preparing a crystalline solid form of a compound having the following Structure (I): 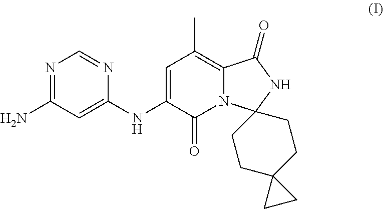 [SMILES: Cc1cc(Nc2cc(N)ncn2)c(=O)n2c1C(=O)NC21CCC2(CC2)CC1]
or a tautomer thereof, the method comprising steps of:
(i) providing Structure (I);
(ii) contacting Structure (I) with one or more suitable solvents (e.g., n-heptane, ethyl acetate, isopropyl acetate, methyl isobutyl ketone, 2-propanol, methyl ethyl ketone, acetone, ethanol, tert-butyl methyl ether, 2-methyl-1-propanol, cyclohexane, methanol, toluene, tetrahydrofuran, acetonitrile, water, dimethylsulfoxide, or a combination thereof); and
(iii) isolating the crystalline solid form,
the method optionally comprising a step of heating or cooling the mixture of Structure (I) in a suitable solvent or a solid precipitate isolated therefrom.

38. A method of preparing a salt form of a compound having the following Structure (I): 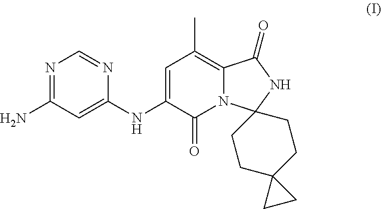 [SMILES: Cc1cc(Nc2cc(N)ncn2)c(=O)n2c1C(=O)NC21CCC2(CC2)CC1]
or a tautomer thereof, the method comprising steps of:
(i) providing Structure (I);
(ii) contacting Structure (I) with a co-former selected from hydrochloric acid, hydrobromic acid, sulfuric acid, acetic acid, maleic acid, fumaric acid, phosphoric acid, citric acid, p-toluenesulfonic acid, methanesulfonic acid, benzenesulfonic acid, tartaric acid, succinic acid, and malic acid; and
(iii) isolating the salt form.

39. A method for treating, preventing, or mitigating the effects of a migraine or symptoms related to a migraine, the method comprising administering a therapeutically effective amount of the solid form of any one of claims 1-28 or a pharmaceutical composition of any one of claims 29-32.

40. A method for treating, preventing, or mitigating the effects of a disease associated with aberrant MNK activity in a mammal in need thereof, comprising administering to the mammal a therapeutically effective amount of a solid form of any one of claims 1-28, or a pharmaceutical composition of any one of claims 29-32.

41. A method for treating, preventing, or mitigating the effects of neuropathic pain, Lupus, viral infection-induced pain, COVID-19 related acute respiratory distress syndrome (ARDS), nonalcoholic fatty liver disease (NAFLD), high fat diet induced obesity, Alzheimer's disease, or Fragile X syndrome in a mammal in need thereof, comprising administering to the mammal a therapeutically effective amount of a solid form of any one of claims 1-28, or a pharmaceutical composition of any one of claims 29-32.
